SEQUENCE LISTING (40 sequences)

SEQ ID NO: 1 - protein, 116 aa, synthetic construct
  VARIANT 66: note = X is N or G
  VARIANT 88: note = X is N or D
  VARIANT 95: note = X is D or H
  VARIANT 98: note = X is E or D
  VARIANT 113..116: note = This portion of the sequence is optional
MPTTTTKVDIAAFDPDKDGTIHLKDALAAGSAAFDKLDPDKDGTLHAKDLKGRVSEADLKKLDPDXDGTLHKKDYLAAVEAQFKAAXPDNDGTIXARXLASPAGSALVNLIRGSGC

SEQ ID NO: 2 - protein, 112 aa, synthetic construct
MPTTTTKVDIAAFDPDKDGTIHLKDALAAGSAAFDKLDPDKDGTLHAKDLKGRVSEADLKKLDPDNDGTLHKKDYLAAVEAQFKAANPDNDGTIDARELASPAGSALVNLIR

SEQ ID NO: 3 - protein, 112 aa, synthetic construct
MPTTTTKVDIAAFDPDKDGTIHLKDALAAGSAAFDKLDPDKDGTLHAKDLKGRVSEADLKKLDPDGDGTLHKKDYLAAVEAQFKAANPDNDGTIDARELASPAGSALVNLIR

SEQ ID NO: 4 - protein, 112 aa, synthetic construct
MPTTTTKVDIAAFDPDKDGTIHLKDALAAGSAAFDKLDPDKDGTLHAKDLKGRVSEADLKKLDPDGDGTLHKKDYLAAVEAQFKAADPDNDGTIHARDLASPAGSALVNLIR

SEQ ID NO: 5 - protein, 112 aa, synthetic construct
MPTTTTKVDIAAFDPDKDGTIHLKDALAAGSAAFDKLDPDKDGTLHAKDLKGRVSEADLKKLDPDNDGTLHKKDYLAAVEAQFKAADPDNDGTIHARDLASPAGSALVNLIR

SEQ ID NO: 6 - protein, 229 aa, synthetic construct
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAAR

SEQ ID NO: 7 - protein, 241 aa, synthetic construct
ELMVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 8 - protein, 582 aa, synthetic construct
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMPTTTTKVDIAAFDPDKDGTIHLKDALAAGSAAFDKLDPDKDGTLHAKDLKGRVSEADLKKLDPDNDGTLHKKDYLAAVEAQFKAANPDNDGTIDARELASPAGSALVNLIRELMVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 9 - protein, 582 aa, synthetic construct
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMPTTTTKVDIAAFDPDKDGTIHLKDALAAGSAAFDKLDPDKDGTLHAKDLKGRVSEADLKKLDPDGDGTLHKKDYLAAVEAQFKAANPDNDGTIDARELASPAGSALVNLIRELMVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 10 - protein, 582 aa, synthetic construct
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMPTTTTKVDIAAFDPDKDGTIHLKDALAAGSAAFDKLDPDKDGTLHAKDLKGRVSEADLKKLDPDGDGTLHKKDYLAAVEAQFKAADPDNDGTIHARDLASPAGSALVNLIRELMVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 11 - protein, 582 aa, synthetic construct
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMPTTTTKVDIAAFDPDKDGTIHLKDALAAGSAAFDKLDPDKDGTLHAKDLKGRVSEADLKKLDPDNDGTLHKKDYLAAVEAQFKAADPDNDGTIHARDLASPAGSALVNLIRELMVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 12 - DNA, 20 bp, synthetic construct
  source 1..20: mol_type = other DNA
atgccatagcatttttatcc

SEQ ID NO: 13 - DNA, 18 bp, synthetic construct
  source 1..18: mol_type = other DNA
gatttaatctgtatcagg

SEQ ID NO: 14 - DNA, 18 bp, synthetic construct
  source 1..18: mol_type = other DNA
caaccactacctgagcac

SEQ ID NO: 15 - DNA, 20 bp, synthetic construct
  source 1..20: mol_type = other DNA
taatacgactcactataggg

SEQ ID NO: 16 - DNA, 19 bp, synthetic construct
  source 1..19: mol_type = other DNA
gctagttattgctcagcgg

SEQ ID NO: 17 - DNA, 21 bp, synthetic construct
  source 1..21: mol_type = other DNA
cgcaaatgggcggtaggcgtg

SEQ ID NO: 18 - DNA, 18 bp, synthetic construct
  source 1..18: mol_type = other DNA
tagaaggcacagtcgagg

SEQ ID NO: 19 - DNA, 25 bp, synthetic construct
  source 1..25: mol_type = other DNA
aatacatatgccaactacgactacc

SEQ ID NO: 20 - DNA, 29 bp, synthetic construct
  source 1..29: mol_type = other DNA
aatagaattcttaacgaattaagttgacc

SEQ ID NO: 21 - DNA, 50 bp, synthetic construct
  source 1..50: mol_type = other DNA
gctgtccaccagtcatgctagccatagatcctttctcctctttcagatcc

SEQ ID NO: 22 - DNA, 50 bp, synthetic construct
  source 1..50: mol_type = other DNA
cggcatggacgagctatacaagtaataaggatctccaggcatcaaataaa

SEQ ID NO: 23 - DNA, 50 bp, synthetic construct
  source 1..50: mol_type = other DNA
tttatttgatgcctggagatccttattacttgtatagctcgtccatgccg

SEQ ID NO: 24 - DNA, 50 bp, synthetic construct
  source 1..50: mol_type = other DNA
ggatctgaaagaggagaaaggatctatggctagcatgactggtggacagc

SEQ ID NO: 25 - DNA, 58 bp, synthetic construct
  source 1..58: mol_type = other DNA
gaacagctcctcgcccttgctcaccatgctcggtaccaagcttaagtttaaacgctag

SEQ ID NO: 26 - DNA, 57 bp, synthetic construct
  source 1..57: mol_type = other DNA
cactctcggcatggacgagctatacaagtaagaattctgcagatatccacacagtgg

SEQ ID NO: 27 - DNA, 53 bp, synthetic construct
  source 1..53: mol_type = other DNA
ggagcggctcccgaagcctcatgctcggtaccaagcttaagtttaaacgctag

SEQ ID NO: 28 - DNA, 42 bp, synthetic construct
  source 1..42: mol_type = other DNA
gtttaaacttaagcttggtaccgagcatgaggcttcgggagc

SEQ ID NO: 29 - DNA, 51 bp, synthetic construct
  source 1..51: mol_type = other DNA
ctgtgctggatatctgcagaattcttacttgtacagctcgtccatgccgag

SEQ ID NO: 30 - protein, 116 aa, synthetic construct
MPTTTTKVDIAAFDPDKDGTIHLKDALAAGSAAFDKLDPDKDGTLHAKDLKGRVSEADLKKLDPDNDGTLHKKDYLAAVEAQFKAADPDNDGTIHARDLASPAGSALVNLIRGSGC

SEQ ID NO: 31 - DNA, 351 bp, synthetic construct
  source 1..351: mol_type = other DNA
atgccaactacgactaccaaagttgatatcgcggcgtttgacccggacaaagatgggaccatccacctgaaagacgctttggcggcaggttccgcggccttcgacaagttggacccggataaagatggtactctgcacgccaaagacctgaagggccgcgtgtctgaggcagaccttaagaagctggacccggacaatgacggaaccctgcacaagaaagactacttagcagcggtagaggcacagtttaaggccgctgaccctgacaacgatggcactattcacgcccgtgaccttgcaagcccagcggggtcggccctggtcaacttaattcgtggcagcggctgctaa

SEQ ID NO: 32 - DNA, 1749 bp, synthetic construct
  source 1..1749: mol_type = other DNA
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgccaactacgactaccaaagttgatatcgcggcgtttgacccggacaaagatgggaccatccacctgaaagacgctttggcggcaggttccgcggccttcgacaagttggacccggataaagatggtactctgcacgccaaagacctgaagggccgcgtgtctgaggcagaccttaagaagctggacccggacaatgacggaaccctgcacaagaaagactacttagcagcggtagaggcacagtttaaggccgctaaccctgacaacgatggcactattgacgcccgtgaacttgcaagcccagcggggtcggccctggtcaacttaattcgtgagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctatacaagtaa

SEQ ID NO: 33 - DNA, 1749 bp, synthetic construct
  source 1..1749: mol_type = other DNA
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgccaactacgactaccaaagttgatatcgcggcgtttgacccggacaaagatgggaccatccacctgaaagacgctttggcggcaggttccgcggccttcgacaagttggacccggataaagatggtactctgcacgccaaagacctgaagggccgcgtgtctgaggcagaccttaagaagctggacccggacggtgacggaaccctgcacaagaaagactacttagcagcggtagaggcacagtttaaggccgctaaccctgacaacgatggcactattgacgcccgtgaacttgcaagcccagcggggtcggccctggtcaacttaattcgtgagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctatacaagtaa

SEQ ID NO: 34 - DNA, 1749 bp, synthetic construct
  source 1..1749: mol_type = other DNA
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgccaactacgactaccaaagttgatatcgcggcgtttgacccggacaaagatgggaccatccacctgaaagacgctttggcggcaggttccgcggccttcgacaagttggacccggataaagatggtactctgcacgccaaagacctgaagggccgcgtgtctgaggcagaccttaagaagctggacccggacggtgacggaaccctgcacaagaaagactacttagcagcggtagaggcacagtttaaggccgctgaccctgacaacgatggcactattcacgcccgtgaccttgcaagcccagcggggtcggccctggtcaacttaattcgtgagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctatacaagtaa

SEQ ID NO: 35 - DNA, 1749 bp, synthetic construct
  source 1..1749: mol_type = other DNA
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgccaactacgactaccaaagttgatatcgcggcgtttgacccggacaaagatgggaccatccacctgaaagacgctttggcggcaggttccgcggccttcgacaagttggacccggataaagatggtactctgcacgccaaagacctgaagggccgcgtgtctgaggcagaccttaagaagctggacccggacaatgacggaaccctgcacaagaaagactacttagcagcggtagaggcacagtttaaggccgctgaccctgacaacgatggcactattcacgcccgtgaccttgcaagcccagcggggtcggccctggtcaacttaattcgtgagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctatacaagtaa

SEQ ID NO: 36 - DNA, 339 bp, synthetic construct
  source 1..339: mol_type = other DNA
atgccaactacgactaccaaagttgatatcgcggcgtttgacccggacaaagatgggaccatccacctgaaagacgctttggcggcaggttccgcggccttcgacaagttggacccggataaagatggtactctgcacgccaaagacctgaagggccgcgtgtctgaggcagaccttaagaagctggacccggacaatgacggaaccctgcacaagaaagactacttagcagcggtagaggcacagtttaaggccgctaaccctgacaacgatggcactattgacgcccgtgaacttgcaagcccagcggggtcggccctggtcaacttaattcgttaa

SEQ ID NO: 37 - DNA, 339 bp, synthetic construct
  source 1..339: mol_type = other DNA
atgccaactacgactaccaaagttgatatcgcggcgtttgacccggacaaagatgggaccatccacctgaaagacgctttggcggcaggttccgcggccttcgacaagttggacccggataaagatggtactctgcacgccaaagacctgaagggccgcgtgtctgaggcagaccttaagaagctggacccggacggtgacggaaccctgcacaagaaagactacttagcagcggtagaggcacagtttaaggccgctaaccctgacaacgatggcactattgacgcccgtgaacttgcaagcccagcggggtcggccctggtcaacttaattcgttaa

SEQ ID NO: 38 - DNA, 339 bp, synthetic construct
  source 1..339: mol_type = other DNA
atgccaactacgactaccaaagttgatatcgcggcgtttgacccggacaaagatgggaccatccacctgaaagacgctttggcggcaggttccgcggccttcgacaagttggacccggataaagatggtactctgcacgccaaagacctgaagggccgcgtgtctgaggcagaccttaagaagctggacccggacggtgacggaaccctgcacaagaaagactacttagcagcggtagaggcacagtttaaggccgctgaccctgacaacgatggcactattcacgcccgtgaccttgcaagcccagcggggtcggccctggtcaacttaattcgttaa

SEQ ID NO: 39 - DNA, 339 bp, synthetic construct
  source 1..339: mol_type = other DNA
atgccaactacgactaccaaagttgatatcgcggcgtttgacccggacaaagatgggaccatccacctgaaagacgctttggcggcaggttccgcggccttcgacaagttggacccggataaagatggtactctgcacgccaaagacctgaagggccgcgtgtctgaggcagaccttaagaagctggacccggacaatgacggaaccctgcacaagaaagactacttagcagcggtagaggcacagtttaaggccgctgaccctgacaacgatggcactattcacgcccgtgaccttgcaagcccagcggggtcggccctggtcaacttaattcgttaa

SEQ ID NO: 40 - protein, 4 aa, synthetic construct
GSGC

(19) United States
(12) Patent Application Publication
(10) Pub. No.: US 2026/0042804 A1
(43) Pub. Date: Feb. 12, 2026

(54) PROTEIN-BASED MATERIAL FOR RECOVERY AND SEPARATION OF TRANSITION METALS

(71) Applicant: The Penn State Research Foundation, University Park, PA (US)

(72) Inventors: Joseph Alfred COTRUVO, Jr., University Park, PA (US); Jennifer PARK, University Park, PA (US)

(21) Appl. No.: 19/101,543

(22) PCT Filed: Aug. 9, 2023
(86) PCT No.: PCT/US2023/071963

§ 371 Date: Feb. 5, 2025

Related U.S. Application Data
(60) Provisional application No. 63/370,915, filed on Aug. 9, 2022.

(51) Int. Cl. C07K 14/195; A61K 49/00; G01N 33/542; G01N 33/84

(52) U.S. Cl. CPC C07K 14/195; A61K 49/0045; G01N 33/542; G01N 33/84

(57) ABSTRACT

Provided are proteins and protein-based sensors for detecting Mn^{II}. The proteins may have the following sequence: Z¹-MPTTTTKVDIAAFDPDKDGTIHLKDALAAGSAAFDKLDPD-KDGTLHAKDLKGRVSEADLKKLDPDX¹DGTLHKKDYLAAVEAQFKAAX²PDNDGTIX³ARX⁴LASPAGSALVNLIR-X⁵-Z² (SEQ ID NO:1), where Z¹ and Z² correspond to a FRET pair, X¹ is N or G, X² is N or D, X³ is D or H, X⁴ is E or D, and X⁵ is optional and is the sequence GSGC (SEQ ID NO:40) and when X⁵ is present, then Z¹ and Z² are absent. Z¹ and Z² are optional. Also provided are methods of using the proteins to detect and separate Mn^{II}. Also provided are compositions, kits, and devices.

### CROSS REFERENCE TO RELATED APPLICATIONS

This application claims priority to U.S. Provisional Patent Application 63/370,915, filed Aug. 9, 2022, the disclosure of which is incorporated herein by reference.

### STATEMENT REGARDING FEDERALLY SPONSORED RESEARCH

This invention was made with government support under Grant No. GM138308 awarded by the National Institutes of Health. The Government has certain rights in the invention.

### SEQUENCE LISTING

The instant application contains a Sequence Listing, which has been submitted electronically in XML format and is hereby incorporated by reference in its entirety. The XML copy, created Aug. 9, 2023, is named “074339_00249_ST26.xml” and is 50,198 bytes.

### BACKGROUND OF THE DISCLOSURE

Manganese is an essential metal ion across all domains of life. In prokaryotes, manganese is a cofactor for enzymes required for many bacteria to thrive under low-iron conditions, including ribonucleotide reductases for DNA synthesis and repair, and therefore is tightly regulated and closely tied to bacterial pathogenesis. Manganese is also integral to oxidative defense, in bacterial and mitochondrial superoxide dismutases as well as in non-enzymatic chemistry of low-molecular-weight Mn^{II}-phosphate complexes, Mn^{II} being the predominant oxidation state of manganese in the cell. Manganese homeostasis also is linked to neuronal development and function; accumulation of excess manganese in the brain induces parkinsonian-like motor disease, whereas reduced manganese levels are associated with Huntington's disease. Manganese is an essential component of the photosynthetic oxygen-evolving cluster, motivating study of its trafficking in plants and algae. However, understanding of manganese physiology has lagged behind that of other essential metals, with the first mammalian Mn^{II} transporters only recently having been discovered—in part because of the paucity of methods to visualize Mn^{II} concentration, localization, speciation, and dynamics within cells.

A large portion of the metal ions in a cell exist in exchangeable pools, relatively weakly bound to cellular ligands that buffer their “free” concentrations; cells attempt to regulate these free concentrations for a particular metal ion within a relatively narrow range. Understanding of free metal concentrations and how they change with time under various conditions is a major challenge, however, because of the dynamic nature of the pools and because any sensor must be able to exchange metal ions with those pools yet not alter their size significantly. In the case of Mn^{II}, the development of such tools is also hindered by its inherent coordination chemistry, as Mn^{II} is the lowest ion in the Irving-Williams series, a ranking of first-row divalent transition metal ions according to the stabilities of their metal-ligand complexes. In other words, other biologically essential transition metals—especially abundant Fe^{II} and Zn^{II}—readily outcompete Mn^{II} for ligand binding. Millimolar free concentrations of Mg^{II} and (in eukaryotes) nanomolar to micromolar Ca^{II} are also important competitors in cells.

Consequently, the design of fluorescent sensors that selectively respond to Mn^{II} is a major, unmet challenge. Others have developed a BAPTA-based fluorescent sensor for Mn^{II} that has good selectivity against Ca^{II} and Mg^{II} but interference from Fe^{II}, as well as a displacement-based strategy that requires co-addition of Cd^{II} to cells. Others reported a boron dipyrromethene (BODIPY)-based fluorescent sensor that recognizes Mn^{II} using a penta-aza macrocycle with pendant methyl ester arms. The BODIPY moiety is lipophilic, and the sensor stains lipid-rich areas of the cell, including the Golgi, which is a site of manganese accumulation in mammalian cells. Efforts to increase this sensor's water solubility instead yielded a sensor that responded to Hg^{II}, not Mn^{II}. Other reported small-molecule sensors for Mn^{II} are similarly too hydrophobic for in-cell application. An ex-vivo method for Mn^{II} quantification was reported by others, using an ionophore to selectively extract Mn^{II} from cells, followed by quantification using the non-specific fluorophore Fura-2. However, this approach does not allow for real-time or subcellular Mn^{II} quantification, and it is not known what Mn^{II} pools are accessed by this molecule. Manganese often challenges the limits of detection of synchrotron-based methods such as X-ray fluorescence microscopy. which also require cell fixation. Therefore, the few available approaches for Mn^{II} sensing have crucial limitations.

In 2018, lanmodulin (LanM), the first natural, highly selective chelator for lanthanide (Ln^{III}) ions (FIG. 1) was reported. This 12-kDa protein is predominantly unfolded in its metal-unbound state, but it undergoes a large conformational response to an ordered state upon binding Ln^{III} ions to three of its four EF hands (EF1, EF2, and EF3), while EF4 does not bind metal ions tightly. EF hands are helix-loop-helix motifs with 12-residue carboxylate-rich loops that bind metals, often inducing a conformational change in the protein. Whereas most EF-hand proteins natively respond to Ca^{II}, LanM responds to trivalent lanthanides and actinides with picomolar apparent K_{d}s (K_{d,app}), but is poorly responsive to Ca^{II} and other divalent metal ions like Mn^{II}. LanM's enormous (>10⁸-fold) selectivity for f-elements appears to result from those metal ions optimally stabilizing the protein's extensively hydrophobically packed folded structure, whereas other metal ions induce a distinct protein conformation that is less stable. Inspired by numerous genetically encoded sensors where conformational change is leveraged for a change in Forster resonance energy transfer (FRET), the ECFP-citrine FRET pair was appended to LanM to create a genetically encoded sensor (LaMP1) for selective detection of Ln^{III} ions and applied it to monitor lanthanide uptake kinetics and localization in methylotrophic bacteria. For several reasons, LaMP1 was considered as an unconventional but promising scaffold for evolution of a Mn^{II} sensor. Previous attempts to alter selectivity of EF hands for other metal ions, including to create genetically encoded fluorescent sensors for Mg^{II} and actinide-binding peptides, have led to rather low affinities or modest selectivity changes, with the exception of the lanthanide binding tag. Indeed, examples of functional metal ion sensors created by re-engineering a protein's metal selectivity are rare, and none exist for Mn^{II}.

### SUMMARY OF THE DISCLOSURE

Virtually no chemical biology tools exist for real-time imaging of manganese(II) in cells. Such tools could help to answer important questions as to how manganese functions in oxidative defense, both when protein-bound as an enzyme cofactor and unbound in the labile manganese pool. Described herein is a lanthanide-binding protein that can be re-engineered to respond to manganese with strong selectivity over the most important interfering metals in cells (magnesium, iron, and calcium). This genetically encoded fluorescent sensor reports manganese fluxes in bacterial cells in real time, laying the foundation for a new approach to studying manganese physiology. More broadly, it suggests general strategies for re-engineering non-native metal selectivity into proteins for wide-ranging applications, including metal separations.

In an aspect, the present disclosure provides proteins that bind metals (e.g., manganese).

The present disclosure provides proteins/peptides suitable for binding manganese. The proteins/peptides may be modified to comprise a FRET pair (e.g., a FRET donor on one terminus and a FRET acceptor on the other terminus).

In an aspect, the present disclosure provides proteins/peptides. The family of proteins/peptides may be referred to as MnLanM followed by a number, where the number refers to the specific family member. The proteins/peptides may further comprise a FRET pair. When the proteins/peptides comprise a FRET pair, the family is referred to as MnLaMP followed by a number, where the number refers to the specific family member. Each numbered MnLaMP protein/peptide comprises or includes the same-numbered MnLanM and a FRET pair. For example, MnLaMP1 comprises MnLanM1.

For example, a protein/peptide of the present disclosure has the following sequence:

(SEQ ID NO: 1)
Z¹-MPTTTTKVDIAAFDPDKDGTIHLKDALAAGSAAFDKLDPDKDGTLH
AKDLKGRVSEADLKKLDPDX¹DGTLHKKDYLAAVEAQFKAAX²PDNDGT
IX³ARX⁴LASPAGSALVNLIR-X⁵-Z²,

where Z¹ and Z² correspond to a FRET pair, X¹ is N or G, X² is N or D, X³ is D or H, X⁴ is E or D, and X⁵ is optional and is the peptide sequence GSGC (SEQ ID NO:40). Z¹ may be a FRET acceptor or FRET donor. When Z¹ is a FRET acceptor, Z² is a FRET donor. Z² may be a FRET acceptor or FRET donor. When Z² is a FRET acceptor, Z¹ is a FRET donor. Z¹ and Z² are optional. When X⁵ is present, then Z¹ and Z² are absent. In various examples, with the exception of Z¹, Z², X¹, X², X³, X⁴, or X⁵ a protein of the present disclosure has 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity with the base protein or peptide sequence.

In an aspect, the present disclosure provides compositions. The composition may comprise a protein/peptide of the present disclosure and a pharmaceutically acceptable carrier.

In an aspect, the present disclosure provides methods of using proteins/peptides of the present disclosure. The methods may comprise binding Mn^{II} to proteins/peptides of the present disclosure. Following binding, the presence of Mn^{II} can be determined.

In an aspect, the present disclosure provides devices. The device comprises one or more proteins of the present disclosures.

In an aspect, the present disclosure provides kits. The kits may provide one or more proteins of the present disclosure and/or one or more devices of the present disclosure. The kit may include instructions for use of the proteins or devices.

### BRIEF DESCRIPTION OF THE FIGURES

For a fuller understanding of the nature and objects of the disclosure, reference should be made to the following detailed description taken in conjunction with the accompanying figures.

FIG. 1. Structure of lanmodulin and a representative metal-binding site. (A) NMR solution structure of Y^{III}-bound LanM, with each EF-hand labeled. Y^{III} ions are represented as spheres. (B) Detail of EF2. The residues coordinating in the model are shown as sticks and labeled with their position in the loop. Studies of Tb^{III}-, Eu^{III}-, Gd^{III}-, and Cm^{III}-bound LanM have revealed an average of 2 coordinated solvent molecules per site. Because the protein residues at the metal-binding sites can theoretically provide up to 10-coordination, it is likely that at least one sidechain does not directly coordinate the metal ion. Indeed, recent evidence suggests that D9 hydrogen bonds with coordinated solvent.

FIG. 2. Biochemical and biophysical characterization of MnLaMP1. (A) General concept for MnLaMP1. (B) FRET ratios (F₅₂₇n/F₄₇₈ nm) of MnLaMP1 plotted against free concentrations of Mn^{II}, Ca^{II}, and Mg^{II}, determined by fluorescence titration (λₑₓ=433 nm). Each point represents the mean±SEM for three technical replicates. (C) Solvent coordination in MnLanM probed by ¹⁷C NMR. MnLanM r° plotted as a function of temperature between 1° and 40° C. (circles) and simulated temperature-dependent r° for high spin Mn^{II} complexes (Ao/h=3.33×10⁷ rad/s) with the following q and exchange parameters: (black line) q=2.2, τₘ²⁵=49 ns, ΔH^{≠}=39 kJ/mol. The vertical dashed line corresponds to 50° C. (1000/T=3.1 K⁻¹), above which MnLanM exhibits thermal instability. [MnLanM]=1.22 mM. [Mn^{II} ]=0.248 mM. (D) Plot of K_{eff} of MnLaMP1 versus number of d electrons for first-row transition metal ions and Ca^{II}. Conditions: 30 mM MOPS, 100 mM KCl, pH 7.2, 500 nM MnLaMP1. The data point for Cu^{II} is shown as an open circle, because it was determined for MnLanM1 using CD at pH 6.0, owing to the poor solubility of Cu(OH)₂ at pH 7.2 and Cu^{II}-induced fluorescence quenching.

FIG. 3. Characterization of MnLaMP1 allows for selective monitoring of Mn^{II} homeostasis in real-time in E. coli. (A) FRET ratios monitored by MnLaMP1 in WT E. coli (BW25113) exposed to 0, 100, 250, or 500 μM MnCl₂. Data points are mean±SEM for 5 biological replicates. The estimated free Mn^{II} concentration values on the right axis were calculated by translating the in-cell FRET ratios (left axis) to the Mn^{II} titration data in FIG. 2B. (B) Metal selectivity of MnLaMP1 in WT E. coli. Cells were exposed to 500 μM CaCl₂), MnCl₂, ferric ammonium citrate, or ZnSO₄, or 3 mM MgCl₂. FRET ratios were determined and shown as mean±SEM for 5 biological replicates. (C) Quantification of total cellular manganese by ICP-MS in E. coli WT and ΔmntP. Data points are mean±SEM for 4 and 5 biological replicates, respectively, in each cell type. Total Mn levels in ppb (FIG. 23) were normalized to cell number by cell counting. The ICP-MS data in control cells (no Mn added) agrees well with prior studies; the results in Mn-exposed cells are ˜4-fold higher, which may be a result of our longest timepoint being 1 h rather than the 2.5 h used in prior studies. (D) Kinetic analysis of intracellular free Mn^{II} concentrations in E. coli knockout strains of genes involved in Mn homeostasis: WT, ΔmntP, ΔmntH, and ΔmntR. Cells were exposed to 500 μM MnCl₂. Data points are mean±SEM for 3, 4, or 5 biological replicates for ΔmntR, ΔmntH, and ΔmntP, respectively. *p<0.05 vs. 0 μM (panels A, B, and C) or vs. WT (panel D) by ANOVA. The bracket in D indicates that all three mutants are significant vs. WT. The baseline FRET ratios of the mutants were not significantly different from WT at t=0.

FIG. 4. In vitro and in-cell characterization of MnLaMP2. A) In vitro fluorescence response of MnLaMP2 to Mn^{II}, Ca^{II}, and Mg^{II}. Compare with FIG. 2B for MnLaMP1; K_{eff} values in Table 2. Each point represents mean±SEM for 3 technical replicates. B) MnLaMP2 is highly selective for Mn^{II} over other biological relevant metal ions (Ca^{II}, Fe^{II}, Mg^{II}, and Zn^{II}) in WT E. coli. Data points are mean±SEM for 3 biological replicates (*p<0.05 for 0 M vs. M^{II} by ANOVA). C) Application of MnLaMP2 to monitor Mn^{II} dynamics in E. coli WT and ΔmntR at 37° C. Data points are mean±SEM for 3 biological replicates (*p<0.05 for ±Mn in each cell type by ANOVA). Free Mn^{II} concentration estimation (right axis) used FIG. 32.

FIG. 5. A cartoon depicting the FRET activity of a protein of the present disclosure.

FIG. 6. Structures of selected manganese binding sites in biology. (A) Mn^{II}-binding site (MnA) from the x-ray crystal structure of MntR (PDB code: 2F5D), showing the distorted pentagonal bipyramidal geometry of the Mn^{II} ion. The Mn^{II} is coordinated by the sidechains of His77 and Glu99 in monodentate fashion, Glu11 and Glu102 in bidentate fashion, and by a solvent molecule. (B) Detail of metal-binding sites in the x-ray crystal structure of the Cd^{II} and Mg^{II}-bound form of the ykoY-mntP Mn^{II} riboswitch chimera (PDB code: 6CC3). Cd^{II} is coordinated by A41, C40, A39, U45 and one solvent molecule. Mg^{II} (dark blue sphere) is coordinated by G9, A39, U45 and three solvent molecules.

FIG. 7. Comparison of EF1 from the x-ray crystal structures of the N-terminal lobe of CaM, in the presence of either Ca^{II} (green sphere) or Mn^{II} (purple spheres). (A) Ca^{II} is coordinated by the sidechains of D20, D22, and D24 (monodentate), the sidechain of E31 (bidentate), the backbone carbonyl of T26, and one solvent molecule (red sphere), resulting in a pentagonal bipyramidal geometry (PDB code: 4CLN). (B) Mn^{II} is coordinated by the same amino acids as the Ca^{II}-bound EF hand, except a solvent molecule occupies the place of E31, yielding octahedral geometry. That coordinated solvent is connected to E31 via a hydrogen bonding network mediated by an additional solvent molecule (PDB 3UCT).

FIG. 8. Emission spectra showing FRET changes during in vitro titration of MnLaMP1 with Mn^{II}. Free Mn^{II} concentrations for the plotted curves are: 0, 3 μM, 10 μM, 30 μM, 70 μM, 300 M, and 1 mM. Conditions: 30 mM MOPS, 100 mM KCl, pH 7.2, 500 nM MnLaMP1.

FIG. 9. Fitting of the FRET ratio data for MnLaMP1 (FIG. 2B) using a method known in the art to estimate the apparent K_{d} for Mn^{II} binding to MnLaMP1. The fit used points at free concentrations of 0.6 to 150 μM; the minor, lower-affinity phase could not be analyzed using this approach and was ignored. The resulting K_{d,app} is 15±1 μM with n=1.3±0.1 (note the excellent agreement with the ITC analysis of MnLanM in Table 7). The multiphase responses of many of the sensors constructed herein and the more difficult implementation of this analysis in cells led us to simply utilize the K_{eff} values in further work, which does not affect our analysis of in-cell free Mn^{II} concentrations. Data represent mean SEM of 3 technical replicates.

FIG. 10. Determination of K_{eff}, Hill coefficient (n), and FRET response for MnLaMP1 (0.5 μM), using unbuffered and/or buffered metal solutions (Fe^{II}, Co^{II}, Ni^{II}, Zn^{II}, and La^{III}). Plots with (A) Fe^{II}, (B) Co^{II} and (C) Ni^{II} were fitted to the Hill equation with one set of interacting sites. Plots with (D) Zn^{II} and (E) La^{III} were fitted to the Hill equation with two sets of interacting sites (the minor response in the 10-100 nM range for Zn^{II} could not be fitted to an additional site). The titrations with Fe^{II} were carried out anaerobically as described in the Experimental Section. K_{eff} of Zn^{II}-MnLaMP1 was determined by combining data from titrations using EGTA-buffered Zn^{II} solutions and unbuffered Zn^{II} solutions. K_{eff} of La^{III}-MnLaMP1 was determined using EDDS-buffered La^{III} titration. K_{eff} values, FRET changes, and n values are presented in Table 2. Conditions: 0.5 μM sensor, 30 mM MOPS, 100 mM KCl, pH 7.2, 25° C. Data represent mean±SEM of 3 technical replicates. Some error bars are not visible because they are smaller than the data markers. (F) Titration of MnLanM1 with unbuffered solutions of Cu^{II}, followed by circular dichroism spectroscopy (molar ellipticity at 222 nm). Conditions: 15 μM MnLanM1, 20 mM MES, 100 mM KCl, pH 6.0, 25° C. Initial experiments to assess Cu^{II} binding to MnLaMP1 resulted in fluorescence quenching, which has been noted previously for Cu^{II} binding to fluorescent proteins. In addition, the solubility of Cu^{II} at pH 7.0 is ˜16 μM (Kₛₚ=1.6×10⁻¹⁹). Therefore, we investigated the Cu^{II}-induced conformational change using CD in MnLanM1 at pH 6.0, under which conditions Cu^{II} is soluble. The data were fitted to the Hill equation with one set of interacting sites, with K_{d,app}=62±4 μM, n=1.6±0.1, and Δ[Θ]=−440 000 deg cm² dmol⁻¹. Uncertainties were calculated by standard deviation from three independent titrations. By way of comparison, the free concentrations of copper (as Cu^{I}) in cells are thought to be in the sub-femtomolar range.

FIG. 11. Response of MnLaMP1 to Na⁺ and K⁺. MnLaMP1 (0.5 M) was assayed in 30 mM MOPS, pH 7.2, supplemented by the indicated concentrations of NaCl or KCl. (A) MnLaMP1 does not respond significantly to K⁺. (B) MnLaMP1 exhibits a slight increase in basal FRET ratio in the presence of Na⁺. Data represent mean±SEM of 3 technical replicates.

FIG. 12. Titration of MnLaMP1 with citrate-buffered solutions of Mn^{II}, followed by circular dichroism spectroscopy (molar ellipticity at 222 nm). The data were fitted to the Hill equation with one set of interacting sites, with K_{d,app}=43±2 μM, n=1.9±0.1, and Δ[Θ]=−460 deg cm² dmol⁻¹×10⁻³. (Note that there is an appearance of two binding events but they are not sufficiently distinct to be fitted as such.) Uncertainties were calculated by standard deviation from three independent titrations. Conditions: 30 mM MOPS, 100 mM KCl, 1 mM citrate, pH 7.2, 25° C.

FIG. 13. Characterization of Ala variants at the 12^{th} position of individual EF hands, in order to understand the role of each EF hand in MnLaMP1's response to Mn^{II}. Titrations of MnLaMP1 D46A (EF1), D70A (EF2), and D95A (EF3) with citrate-buffered and unbuffered solutions of (A) Mn^{II} or (B) Co^{II}. Parameters for the fits are provided in Table 6. Although the D95A data appear to be two phases in the case of Mn^{II}, only the one-phase fit converged. Conditions: 0.5 μM sensor, 30 mM MOPS, 100 mM KCl, pH 7.2, 25° C. Data represent mean±SEM of 3 technical replicates. Some error bars are not visible because they are smaller than the data markers.

FIG. 14. Binding of Mn^{II} to MnLanM, studied by isothermal titration calorimetry (ITC) at 25° C. (Top) Representative ITC trace for titration of 60 μM untagged apo MnLanM with 2.6 mM MnCl₂ (45 injections of 0.8 μL 2.6 mM MnCl₂). (Bottom) Thermogram derived from the data above, after correction for heat of dilution, and fitted to a model with two sets of sites, with parameters presented in Table 7. Conditions: 30 mM MOPS, 100 mM KCl, pH 7.2, 25° C.

FIG. 15. Binding of Mn^{II} to MnLanM, studied by isothermal titration calorimetry (ITC) at 30° C. (Top) Representative ITC trace for titration of 60 μM untagged apo MnLanM with 3.0 mM MnCl₂ (45 injections of 0.8 μL 3.0 mM MnCl₂). (Bottom) Thermogram derived from the data above, after correction for heat of dilution, and fitted to a model with two sets of sites, with parameters presented in Table 7. Conditions: 30 mM MOPS, 100 mM KCl, pH 7.2, 30° C.

FIG. 16. Binding of Mn^{II} to MnLanM, studied by isothermal titration calorimetry (ITC) at 37° C. (Top) Representative ITC trace for titration of 60 μM untagged apo MnLanM with 5.0 mM MnCl₂ (45 injections of 0.8 μL 5.0 mM MnCl₂). (Bottom) Thermogram derived from the data above, after correction for heat of dilution, and fitted to a model with two sets of sites, with parameters presented in Table 7. Conditions: 30 mM MOPS, 100 mM KCl, pH 7.2, 37° C.

FIG. 17. van't Hoff plot of ln Kₐ of MnLanM (derived from the exothermic phase of the ITC experiments in FIGS. 14-16, shown in Table 7) versus reciprocal of the temperature. The fitted line (y=7126x−12.89) was used to estimate unbound concentrations of Mn^{II} in NMR experiments.

FIG. 18. Determination of the intracellular concentration of MnLaMP1 when constitutively expressed in E. coli BW25113 (wild type). (A) Standard curve relating concentration of purified MnLaMP1 to fluorescence emission at 528 nm. (B) Full spectra of the lowest three standard concentrations along with spectra of lysates (blue: no EDTA added; red: 1 mM EDTA added to remove the slight response of the sensor to trace metals in the lysate) from MnLaMP1-expressing E. coli. Using the +EDTA sample, intracellular MnLaMP1 concentration was determined to be 20±5 μM (two independent determinations) as described herein.

FIG. 19. Calibration of MnLaMP1 in E. coli BW25113 (wild-type) and ΔmntP. Arrows indicate timepoints at which 1 mM TPEN (to determine Rₘᵢₙ) and 2.5 M 4-BrA23187 and 10 mM Mn^{II} (to determine Rₘₐₓ) were added. Uncertainties represent standard deviation from 3 and 4 independent calibrations in WT and ΔmntP, respectively. Experiments using higher ionophore concentration (10 μM) and higher Mn^{II} concentration (30 mM) yielded similar Rₘₐₓ values (not shown), suggesting that these conditions cause sensor saturation. A) Calibration of MnLaMP1 in E. coli WT. Rₘₐₓ=4.2, Rₘᵢₙ=1.3. B) Calibration of MnLaMP1 in E. coli ΔmntP. Rₘₐₓ=3.2, Rₘᵢₙ=1.1. Phosphate-free MOPS minimal medium was used for these experiments to avoid precipitation of manganese phosphate in the saturating Mn^{II} condition.

FIG. 20. Calibration of MnLaMP1 and MnLaMP2 in E. coli ΔmntR. Arrows indicate timepoints at which 1 mM TPEN (to determine Rₘᵢₙ) and 2.5 M 4-BrA23187 and 10 mM Mn^{II} (to determine Rₘₐₓ) were added. Uncertainties represent standard deviation from 2 independent calibrations. A) MnLaMP1 (Rₘₐₓ=2.7, Rₘᵢₙ=1.1). B) MnLaMP2 (Rₘₐₓ=2.6, Rₘᵢₙ=1.1).

FIG. 21. Fluorescence response of MnLaMP1 to Mn^{II} simulated cellular conditions (0.5 μM MnLaMP1; 5 mM Na⁺ and 95 mM K⁺). The plot was fitted to the Hill equation with two sets of interacting sites. The first phase has K_{eff}=23±2 μM, n=1.2, and F/F₀=3.1. The minor second phase has 1.4±0.5 mM, n=2.0, and F/F₀=1.1. Note the slightly higher baseline (1.66), relative to FIG. 2B (100 mM K⁺). Data represent mean SEM of 3 technical replicates.

FIG. 22. Fluorescence response of MnLaMP1 to Mn^{II} in 300 mM KCl, showing the only slight ionic strength dependence of K_{eff}. The plot was fitted to the Hill equation with two sets of interacting sites. The first phase has K_{eff}=41±2 μM and the K_{eff} for the second phase was not well constrained by the data. Data represent mean±SEM of 3 technical replicates.

FIG. 23. Quantification of total Mn^{II} concentration (ppb) in E. coli WT and ΔmntP. These raw values were used to calculate the concentrations in FIG. 3C based on cell counting results and 3.2 fL for the volume of an E. coli cell (see Experimental Section). Data are mean±SEM from 5 biological replicates.

FIG. 24. Determination of MnLaMP1's metal selectivity for Mn^{II} and Ca^{II} in E. coli BW25113 under different metal levels in the presence of ionophores: (A) 10 mM Mn^{II} and 5 M ionomycin, (B) 500 μM Mn^{II} and 2.5 M 4-BrA23187 (compare with FIG. 19 for 10 mM Mn^{II} and 2.5 μM 4-BrA23187), (C) 500 M Ca^{II} and 5 M ionomycin, and (D) 10 mM Ca(II) and 5 M ionomycin. Phosphate-free MOPS medium was used for this experiment to avoid the precipitation of manganese and calcium phosphates. 4-BrA23187 was used for only Mn^{II} uptake experiments because of its low efficiency for Ca^{II} transport. The Mn^{II} and Ca^{II} transport rates for ionomycin are similar. Uncertainties were determined by standard deviation from 2 independent experiments in WT.

FIG. 25. Application of MnLaMP1 in E. coli WT and knockout strains (ΔmntR, ΔmntP, ΔmntH) for comparison of labile Mn^{II} kinetics. A) In the absence of added Mn^{II}. (B,C,D) Comparison of labile Mn^{II} kinetics between WT and ΔmntP, ΔmntH, and ΔmntR. *p<0.05 (0 M vs. 500 M Mn^{II} in each strain. E) Fitting of the WT kinetic data to a two-phase exponential yields apparent time constants τ₁=2.8±0.7 for Mn^{II} increase and τ₂=18±3 for Mn^{II} decrease. See FIG. 3 legend for experimental conditions.

FIG. 26. Tests of MnLaMP1 in mammalian cells. (A and B) HeLa cells transfected with Golgi-MnLaMP1 were imaged live before and after Mn treatment. FRET ratios from multiple cells were quantified in (B); N=15 cells for before Mn and 26 cells for after Mn. (C) FRET ratios for individual cells from (B) were plotted against time. Mn addition=0 min. (D and E) The cis-Golgi protein GPP130 is degraded upon treatment of cells with manganese and served as a control for Mn^{II} accumulation in the Golgi. GPP130 levels after Mn treatment were detected using immunofluorescence. Levels before Mn were normalized to 1. N=20 cells. Data depicted represent mean±SEM; *, p<0.05 by t-test. Scale bar=10 μm.

FIG. 27. Fluorescence response for the X₄G variants in EF1 and EF2 (0.5 M sensor, 25° C., 100 mM KCl, 30 mM MOPS, pH 7.2). A) MnLaMP1-K38G. B) MnLaMP1-K62G. Fitting parameters are given in Table 9. The data for the N87G variant is shown in FIG. 4A. Data represent mean±SEM of 3 technical replicates.

FIG. 28. Response of MnLaMP1-N87G (MnLaMP2) to Na⁺ and K⁺. MnLaMP2 (0.5 μM) was incubated in 30 mM MOPS, pH 7.2, supplemented by the indicated concentrations of NaCl or KCl. A) Response to K⁺. B) Response to different ratios of Na⁺ and K⁺ with constant ionic strength (100 mM). Data represent mean±SEM of 3 technical replicates.

FIG. 29. Fluorescence response of MnLaMP2 to various metal ions (0.5 M sensor, 25° C., 30 mM MOPS, 100 mM KCl, pH 7.2). Plots for A) Ca^{II}, B) Fe^{II}, and C) Co^{II} were fitted to the Hill equation with one set of sites. The Fe^{II} titration was performed under anaerobic conditions. D) The Zn^{II} titration was fitted to the Hill equation with two sets of interacting sites, but the second phase is not saturated with 10 mM Zn^{II}. E) The La^{III} titration, performed using EDDS-La^{III} solutions, was fitted to the Hill equation with two sets of interacting sites. K_{eff} and n values are reported in Table 2. Data represent mean±SEM of 3 technical replicates.

FIG. 30. Plot of K_{eff} of MnLaMP2 versus d-electron count. Conditions: 30 mM MOPS, 100 mM KCl, pH 7.2, 500 nM MnLaMP1. The data point for Cu^{II} is shown as an open circle, because it was determined for MnLanM2 (not MnLaMP2) using CD spectroscopy at pH 6.0, owing to the poor solubility of Cu(OH)₂ at pH 7.2 (Kₛₚ=1.6×10⁻¹⁹) and Cu^{II}-induced fluorescence quenching.

FIG. 31. Calibration of MnLaMP2 in E. coli BW25113 (wild-type). Arrows indicate timepoints at which 1 mM TPEN (to determine Rₘᵢₙ) and 2.5 M 4-BrA23187 and 10 mM Mn^{II} (to determine Rₘₐₓ) were added. Uncertainties represent standard deviation from 3 independent calibrations. Rₘₐₓ=2.9, Rₘᵢₙ=1.2.

FIG. 32. Characterization of MnLaMP2 in the presence of magnesium (500 M Mg^{II}, 100 mM KCl, 30 mM MOPS, pH 7.2; 0.1 μM MnLaMP2 for points 0.1-1 μM Mn^{II}, 0.5 μM MnLaMP2 for points 3 μM-10 mM Mn^{II}) determined by fluorescence titrations unbuffered Mn^{II} solutions. The fit yielded similar parameters as in the absence of Mg^{II}. For the first phase, K_{eff}=9±1 μM, n=1.6±0.4, and F/F₀=1.8. For the second phase, which was not well defined, K_{eff}=110±90 μM, n=0.5±0.1, and F/F₀=1.1. Data represent mean±SEM of 3 technical replicates.

FIG. 33. Comparison and characterization of MnLaMP variants. (A) Comparison of sequences of MnLanM variants. In vitro characterization of (B) MnLaMP3 and (C) MnLaMP4 by fluorescence titration showing FRET ratio (F_{527 nm}/F_{478 nm}) of each sensor in response to Mn^{II}, Co^{II}, Ni^{II}, and Mg^{II}. Each point represents the mean±SEM for three technical replicates. Some error bars are not visible because they are smaller than the data markers. Free Mn^{II} concentrations were buffered using NTA. Co^{II}, Ni^{II}, and Mg^{II} titrations were unbuffered, due to lower affinities. K_{eff} values, FRET changes, and n values are summarized in Table 4. Conditions: 0.1 μM sensor, 30 mM MOPS, 100 mM KCl, pH 7.2, 25° C. (A) contains portions of SEQ ID NO:2 (labeled MnLanM1), portions of SEQ ID NO:3 (labeled MnLanM2), portions of SEQ ID NO:4 (labeled MnLanM3), and portions of SEQ ID NO:5 (labeled MnLanM4).

FIG. 34. Characterization of MnLanM3 and MnLanM4 at (A) pH 7.2 and (B) pH 5.0 with Mn^{II}, Co^{II}, and Ni^{II} by circular dichroism spectroscopy (molar ellipticity at 222 nm). Mn^{II} was assayed using NTA- and citrate-buffered titrations at pH 7.2 and 5.0, respectively. Co^{II}, Ni^{II}, and Mg^{II} titrations were performed unbuffered. Each point represents the mean±SEM for three technical replicates. K_{d,app} values, ΔΘ, and n values are summarized in Table 5. Conditions: (A) 15 M protein, 30 mM MOPS, 100 mM KCl, pH 7.2, 25° C., and (B) 15 M protein, 20 mM acetate, 100 mM KCl, pH 5.0, 25° C.

FIG. 35. Metal separation using MnLanM variants in the presence of 500 kM Mn^{II}, Co^{II}, and Ni^{II}. (A) Schematic of protein-based metal separation using protein concentrators. (B) Comparison of metal separation among MnLanM variants at pH 5.0 determined by inductively coupled plasma mass spectrometry. M/M₀ indicates the ratio of the metal in retentate to metal initially added. The control samples contained no protein. Data represent mean±error for five replicates for every MnLanM variant. The error was calculated using error propagation. Condition: 100 M protein, ˜500 M MnCl₂, ˜500 M CoCl₂, ˜500 M NiSO₄, 100 mM KCl, 20 mM acetate, pH 5.0. *p<0.05 by ANOVA.

FIG. 36. Breakthrough of MnLanM4 with Mn at pH 5.0. (A) Adsorption with 0.2 mM Mn^{II}. (B) Desorption with 25 mM HCl, pH 1.8. The binding capacity of MnLanM4 is 4 mol Mn^{II}/mL agarose, which is ˜0.9:1 stoichiometry of Mn^{II} per immobilized MnLanM4.

FIG. 37. Two metal separation using immobilized MnLanM4 at pH 5.0 quantified by ICP-MS. Solutions containing equimolar Mn^{II} and Co^{II}, or Mn^{II} and Ni^{II}, were applied to the column as described in the experimental section. Although Ni and Co are not completely rejected by the protein, Mn largely outcompetes them for binding. (A) Adsorption process for Mn^{II}/Co^{II} separation. (B) Desorption process for Mn^{II}/Co^{II} separation. (C) Adsorption process for Mn^{II}/Ni^{II} separation. (D) Desorption process for Mn^{II}/Ni^{II} separation.

FIG. 38. Titration data of untagged MnLanM3 with Mn.

FIG. 39. Titration data of untagged MnLanM3 with Co.

FIG. 40. Titration data of untagged MnLanM3 with Ni.

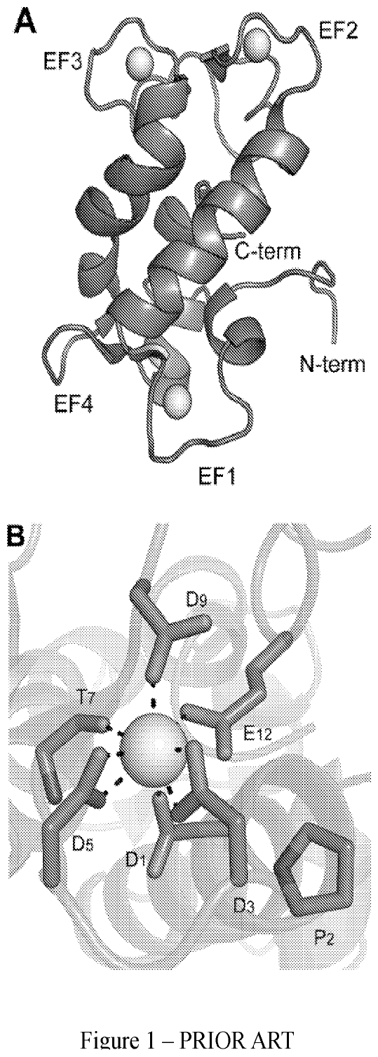

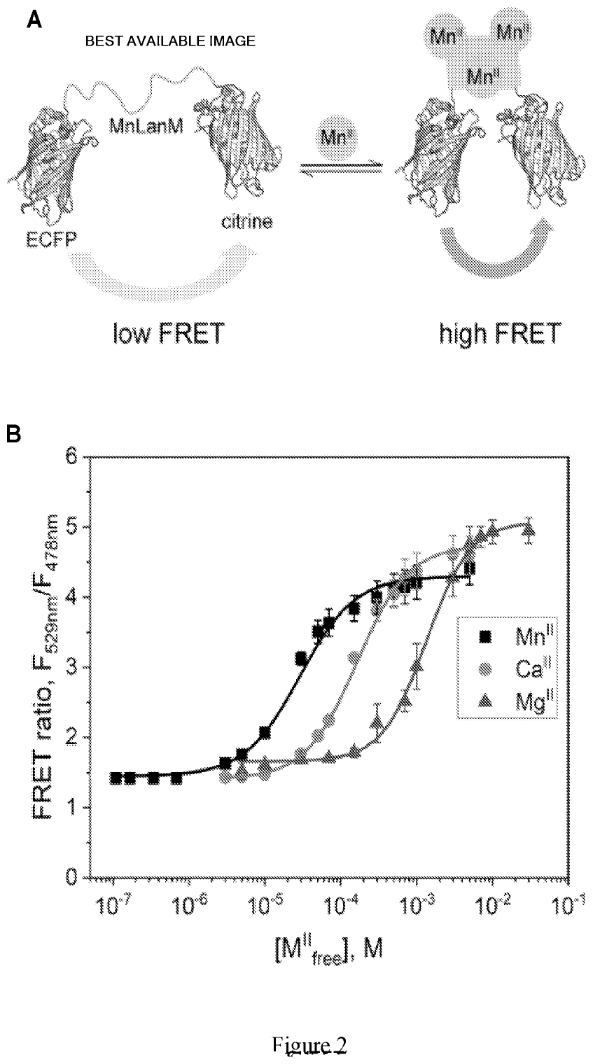

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g., 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%, 0.5% to 2.4%, 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about”, it will be understood that the particular value forms a further disclosure. For example, if the value “about 10” is disclosed, then “10” is also disclosed.

As used herein, the terms “including,” “containing,” and “comprising” are used in their open, non-limiting sense.

As used in this disclosure, the singular forms include the plural forms and vice versa unless the context clearly indicates otherwise.

The articles “a” and “an” are used in this disclosure to refer to one or more than one (i.e., to at least one) of the grammatical object of the article. By way of example, “an element” means one element or more than one element.

As used herein, unless otherwise stated or indicated, “s” refers to second(s), “min” refers to minute(s), and “h” refers to hour(s).

The phrase “therapeutically effective amount” is used herein to mean an amount sufficient to reduce by at least about 15 percent, preferably by at least 50 percent, more preferably by at least 90 percent, and most preferably prevents oxidative stress in the individual. Alternatively, a therapeutically effective amount is sufficient to cause an improvement in a clinically significant condition in the host.

As used herein, “FRET” refers to Forster resonance energy transfer or fluorescence resonance energy transfer. Signals produced by FRET interactions may be determined by fluorescence spectroscopy, methods of which are known in the art.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent, trivalent, and the like, radicals). Illustrative examples of groups include:

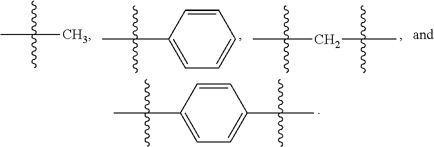

Amino acids and amino acid residues may be referred to herein by either their commonly known three letter symbols or by the one-letter symbols recommended by the IUPAC-IUB Biochemical Nomenclature Commission.

The present disclosure also provides sequences that have homology with the protein or peptides sequences (including antibody sequences) described herein. In various examples, the homologous sequences have at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity with a protein or peptide sequence of the present disclosure.

Examples of hydrophobic amino acid and hydrophobic amino acid residues include, but are not limited to, glycine, alanine, valine, leucine, isoleucine, proline, cysteine, phenylalanine, methionine, tyrosine, and tryptophan.

Virtually no chemical biology tools exist for real-time imaging of manganese(II) in cells. Such tools could help to answer important questions as to how manganese functions in oxidative defense, both when protein-bound as an enzyme cofactor and unbound in the labile manganese pool. Described herein is a lanthanide-binding protein can be re-engineered to respond to manganese with strong selectivity over the most important interfering metals in cells (magnesium, iron, and calcium). This genetically encoded fluorescent sensor reports manganese fluxes in bacterial cells in real time, laying the foundation for a new approach to studying manganese physiology. More broadly, it suggests general strategies for re-engineering non-native metal selectivity into proteins for wide-ranging applications, including metal separations.

In an aspect, the present disclosure provides proteins that bind metals (e.g., manganese). Other metal-binding proteins are disclosed in WO2020051274 and WO2023004333, which are incorporated herein by reference.

The present disclosure provides proteins/peptides suitable for binding manganese. The proteins/peptides may be modified to comprise a FRET pair (e.g., a FRET donor on one terminus and a FRET acceptor on the other terminus).

In an aspect, the present disclosure provides proteins/peptides. The family of proteins/peptides may be referred to as MnLanM followed by a number, where the number refers to the specific family member. The proteins/peptides may further comprise a FRET pair. When the proteins/peptides comprise a FRET pair, the family is referred to as MnLaMP followed by a number, where the number refers to the specific family member. Each numbered MnLaMP protein/peptide comprises the same-numbered MnLanM and a FRET PAIR. For example, MnLaMP1 comprises MnLanM1.

Suitable proteins include the derivatives of M. extorquens LanM protein, or orthologs from other organisms having at least two EF hand motifs, with at least one EF hand motif having at least 3 carboxylate residues, and at least 2 of the EF hand motifs being separated by a space of 10-15 residues. Reference herein will be made generally to “lanmodulin,” “LanM” or “LanM protein” and should be understood to include the wild type and orthologs described herein. “LanM” can include full proteins having one or more LanM units or portions thereof comprising the one or more LanM units. LanM units include at least two EF hand motifs, with at least one EF hand motifs having at least 3 carboxylate residues, and at least 2 of the EF hand motifs being separated by a space of 10-15 residues. For ease of reference, discussion will be made with reference to lanmodulin, LanM or LanM protein and should be understood to include both the full proteins and portions of full proteins having the suitable LanM unit.

For example, a protein/peptide of the present disclosure has the following sequence:

(SEQ ID NO: 1)
Z¹-MPTTTTKVDIAAFDPDKDGTIHLKDALAAGSAAFDKLDPDKDGTLH
AKDLKGRVSEADLKKLDPDX¹DGTLHKKDYLAAVEAQFKAAX²PDNDGT
IX³ARX⁴LASPAGSALVNLIR-X⁵-Z²,

where Z¹ and Z² correspond to a FRET pair, X¹ is N or G, X² is N or D, X³ is D or H, X⁴ is E or D, and X⁵ is optional and is the peptide sequence GSGC (SEQ ID NO:40). Z¹ may be a FRET acceptor or FRET donor. When Z¹ is a FRET acceptor, Z² is a FRET donor. Z² may be a FRET acceptor or FRET donor. When Z² is a FRET acceptor, Z¹ is a FRET donor. Z¹ and Z² are optional. When X⁵ is present, then Z¹ and Z² are absent and vice versa. In various examples, with the exception of Z¹, Z¹, X¹, X², X³, X⁴, and X⁵ a protein of the present disclosure has 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity with the base protein or peptide sequence.

Examples of peptides of the present disclosure include, but are not limited to,

>MnLanM1:
(SEQ ID NO: 2)
MPTTTTKVDIAAFDPDKDGTIHLKDALAAGSAAFDKLDPDKDGTLHAKDL
KGRVSEADLKKLDPDNDGTLHKKDYLAAVEAQFKAANPDNDGTIDARELA
SPAGSALVNLIR;
>MnLanM2:
(SEQ ID NO: 3)
MPTTTTKVDIAAFDPDKDGTIHLKDALAAGSAAFDKLDPDKDGTLHAKDL
KGRVSEADLKKLDPDGDGTLHKKDYLAAVEAQFKAANPDNDGTIDARELA
SPAGSALVNLIR;
>MnLanM3:
(SEQ ID NO: 4)
MPTTTTKVDIAAFDPDKDGTIHLKDALAAGSAAFDKLDPDKDGTLHAKDL
KGRVSEADLKKLDPDGDGTLHKKDYLAAVEAQFKAADPDNDGTIHARDLA
SPAGSALVNLIR;
and
>MnLanM4:
(SEQ ID NO: 5)
MPTTTTKVDIAAFDPDKDGTIHLKDALAAGSAAFDKLDPDKDGTLHAKDL
KGRVSEADLKKLDPDNDGTLHKKDYLAAVEAQFKAADPDNDGTIHARDLA
SPAGSALVNLIR.

Each of the foregoing sequences may comprise a Z¹ group and a Z² group as described herein.

In various examples, the protein is immobilized on a substrate, such as a bead (e.g., agarose bead) or a resin. The protein/peptide may further comprise a Cys-binding region: GSGC (SEQ ID NO:40). Any protein not including a FRET pair may have a Cys-binding region conjugated to its C-terminus. In various examples, MnLanM1, MnLanM2, MnLanM3, or MnLanM4 have a Cys-binding region. For example, the sequence of MnLanM4 having a Cys-binding region conjugated thereto is:

(SEQ ID NO: 30)
MPTTTTKVDIAAFDPDKDGTIHLKDALAAGSAAFDKLDPDKDGTLHAKDL
KGRVSEADLKKLDPDNDGTLHKKDYLAAVEAQFKAADPDNDGTIHARDLA
SPAGSALVNLIRGSGC.

Other substrates may be used and are known in the art. In various embodiments, utilization of immobilization of a protein on a substrate may allow for the selective separation of Mn^{II} from a sample comprising a plurality of metals. In such an example, (i) the protein may be immobilized onto a substrate and then the protein bound to Mn^{II} may selectively be separated from the substrate or (ii) the substrate having the bound protein immobilized thereto may be separated from the sample.

Various FRET pairs may be conjugated to the protein/peptide of the present disclosure. Each FRET pair comprises a donor and a acceptor, which may be referred to Z¹ and Z². Z¹ may be a FRET acceptor or FRET donor. When Z¹ is a FRET acceptor, Z² is a FRET donor. Z² may be a FRET acceptor or FRET donor. When Z² is a FRET acceptor, Z¹ is a FRET donor. In various examples, the FRET pair may be small molecule-based FRET pairs. Examples of small molecule-based FRET pairs include, but are not limited to, Cy3 and Cy5. When the FRET pair is small molecule-based, they may be attached to amino acid residues. In such an example, the Z group would comprise a FRET donor or FRET acceptor conjugated to an amino acid residue. In other examples, the FRET pair may be peptide/protein-based FRET pairs (e.g., fluorescent protein pairs). For example, the FRET pair may be a cyan fluorescent protein (e.g., enhanced cyan fluorescent protein (ECFP)) and yellow fluorescent protein (e.g., citrine)). In various examples, the FRET pair is ECFP and citrine. ECFP may be conjugated to the N-terminus and citrine may be conjugated to the C-terminus of the protein/peptide of the present disclosure. In various embodiments, ECFP is conjugated to the C-terminus and citrine is conjugated to the N-terminus. If the FRET pair is protein/peptide-based, it may comprise additional amino acid residues relative to the native sequence of the fluorescent protein/peptide. Alternatively, the protein/peptide FRET pair may be truncated relative to their native sequences. ECFP as used herein has the following sequence:

(SEQ ID NO: 6)
MVSKGEELFTGVVPILVELDGDVNGHRFSVSGEGEGDATYGKLTLKFICT
TGKLPVPWPTLVTTLTWGVQCFSRYPDHMKQHDFFKSAMPEGYVQERTIF
FKDDGNYKTRAEVKFEGDTLVNRIELKGIDFKEDGNILGHKLEYNYISHN
VYITADKQKNGIKAHFKIRHNIEDGSVQLADHYQQNTPIGDGPVLLPDNH
YLSTQSALSKDPNEKRDHMVLLEFVTAAR.

Citrine as used herein has the following sequence:

(SEQ ID NO: 7)
ELMVSKGEELFTGVVPILVELDGDVNGHKFSVSGEGEGDATYGKLTLKF
ICTTGKLPVPWPTLVTTFGYGLMCFARYPDHMKQHDFFKSAMPEGYVQE
RTIFFKDDGNYKTRAEVKFEGDTLVNRIELKGIDFKEDGNILGHKLEYN
YNSHNVYIMADKQKNGIKVNFKIRHNIEDGSVQLADHYQQNTPIGDGPV
LLPDNHYLSYQSALSKDPNEKRDHMVLLEFVTAAGITLGMDELYK.

In various examples, ECFP may comprise one or more additional amino acid residues or be truncated relative to the sequence disclosed herein. In various examples, citrine may comprise one or more additional amino acid residues or be truncated relative to the sequence disclosed herein.

In various examples, when a protein/peptide of the present disclosure comprises a protein/peptide-based FRET pair, the protein/peptide may have the following sequence or comprise the following sequence:

>MnLaMP1:
(SEQ ID NO: 8)
MVSKGEELFTGVVPILVELDGDVNGHRFSVSGEGEGDATYGKLTLKFICTTGKLPVP
WPTLVTTLTWGVQCFSRYPDHMKQHDFFKSAMPEGYVQERTIFFKDDGNYKTRAE
VKFEGDTLVNRIELKGIDFKEDGNILGHKLEYNYISHNVYITADKQKNGIKAHFKIRH
NIEDGSVQLADHYQQNTPIGDGPVLLPDNHYLSTQSALSKDPNEKRDHMVLLEFVTA
ARMPTTTTKVDIAAFDPDKDGTIHLKDALAAGSAAFDKLDPDKDGTLHAKDLKGRV
SEADLKKLDPDNDGTLHKKDYLAAVEAQFKAANPDNDGTIDARELASPAGSALVNL
IRELMVSKGEELFTGVVPILVELDGDVNGHKFSVSGEGEGDATYGKLTLKFICTTGK
LPVPWPTLVTTFGYGLMCFARYPDHMKQHDFFKSAMPEGYVQERTIFFKDDGNYKT
RAEVKFEGDTLVNRIELKGIDFKEDGNILGHKLEYNYNSHNVYIMADKQKNGIKVNF
KIRHNIEDGSVQLADHYQQNTPIGDGPVLLPDNHYLSYQSALSKDPNEKRDHMVLLE
FVTAAGITLGMDELYK;
>MnLaMP2:
(SEQ ID NO: 9)
MVSKGEELFTGVVPILVELDGDVNGHRFSVSGEGEGDATYGKLTLKFICTTGKLPVP
WPTLVTTLTWGVQCFSRYPDHMKQHDFFKSAMPEGYVQERTIFFKDDGNYKTRAE
VKFEGDTLVNRIELKGIDFKEDGNILGHKLEYNYISHNVYITADKQKNGIKAHFKIRH
NIEDGSVQLADHYQQNTPIGDGPVLLPDNHYLSTQSALSKDPNEKRDHMVLLEFVTA
ARMPTTTTKVDIAAFDPDKDGTIHLKDALAAGSAAFDKLDPDKDGTLHAKDLKGRV
SEADLKKLDPDGDGTLHKKDYLAAVEAQFKAANPDNDGTIDARELASPAGSALVNL
IRELMVSKGEELFTGVVPILVELDGDVNGHKFSVSGEGEGDATYGKLTLKFICTTGK
LPVPWPTLVTTFGYGLMCFARYPDHMKQHDFFKSAMPEGYVQERTIFFKDDGNYKT
RAEVKFEGDTL VNRIELKGIDFKEDGNILGHKLEYNYNSHNVYIMADKQKNGIKVNF
KIRHNIEDGSVQLADHYQQNTPIGDGPVLLPDNHYLSYQSALSKDPNEKRDHMVLLE
FVTAAGITLGMDELYK;
>MnLaMP3:
(SEQ ID NO: 10)
MVSKGEELFTGVVPILVELDGDVNGHRFSVSGEGEGDATYGKLTLKFICTTGKLPVP
WPTLVTTLTWGVQCFSRYPDHMKQHDFFKSAMPEGYVQERTIFFKDDGNYKTRAE
VKFEGDTLVNRIELKGIDFKEDGNILGHKLEYNYISHNVYITADKQKNGIKAHFKIRH
NIEDGSVQLADHYQQNTPIGDGPVLLPDNHYLSTQSALSKDPNEKRDHMVLLEFVTA
ARMPTTTTKVDIAAFDPDKDGTIHLKDALAAGSAAFDKLDPDKDGTLHAKDLKGRV
SEADLKKLDPDGDGTLHKKDYLAAVEAQFKAADPDNDGTIHARDLASPAGSALVN
LIRELMVSKGEELFTGVVPILVELDGDVNGHKFSVSGEGEGDATYGKLTLKFICTTG
KLPVPWPTLVTTFGYGLMCFARYPDHMKQHDFFKSAMPEGYVQERTIFFKDDGNY
KTRAEVKFEGDTLVNRIELKGIDFKEDGNILGHKLEYNYNSHNVYIMADKQKNGIKV
NFKIRHNIEDGSVQLADHYQQNTPIGDGPVLLPDNHYLSYQSALSKDPNEKRDHMVL
LEFVTAAGITLGMDELYK;
or
>MnLaMP4:
(SEQ ID NO: 11)
MVSKGEELFTGVVPILVELDGDVNGHRFSVSGEGEGDATYGKLTLKFICTTGKLPVP
WPTLVTTLTWGVQCFSRYPDHMKQHDFFKSAMPEGYVQERTIFFKDDGNYKTRAE
VKFEGDTLVNRIELKGIDFKEDGNILGHKLEYNYISHNVYITADKQKNGIKAHFKIRH
NIEDGSVQLADHYQQNTPIGDGPVLLPDNHYLSTQSALSKDPNEKRDHMVLLEFVTA
ARMPTTTTKVDIAAFDPDKDGTIHLKDALAAGSAAFDKLDPDKDGTLHAKDLKGRV
SEADLKKLDPDNDGTLHKKDYLAAVEAQFKAADPDNDGTIHARDLASPAGSALVN
LIRELMVSKGEELFTGVVPILVELDGDVNGHKFSVSGEGEGDATYGKLTLKFICTTG
KLPVPWPTLVTTFGYGLMCFARYPDHMKQHDFFKSAMPEGYVQERTIFFKDDGNY
KTRAEVKFEGDTLVNRIELKGIDFKEDGNILGHKLEYNYNSHNVYIMADKQKNGIKV
NFKIRHNIEDGSVQLADHYQQNTPIGDGPVLLPDNHYLSYQSALSKDPNEKRDHMVL
LEFVTAAGITLGMDELYK.

A protein/peptide comprising a protein/peptide-based FRET pair may have 75%, 76%, 77%, 78%, 79%, 80%, 81%, 82%, 83%, 84%, 85%, 86%, 87%8, 88%, 89%, 90%, 91%, 92%, 93%, 94%, 95%, 96%, 97%, 98%, or 99% identity with the foregoing sequences.

A protein/peptide of the present disclosure has several desirable features. For example, a protein/peptide of the present disclosure undergoes a conformational change in response to Mn^{II} with a selectivity that is at least 2 to 30-fold (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30) over other divalent metals (e.g., Ca^{II}, Fe^{II}, Co^{II}, Ni^{II}, Zn^{II}, Mg^{II}). For example, a protein/peptide of the present disclosure displays minimal responsiveness to monovalent ions, such as, for example, Na⁺ and K⁺.

In an aspect, the present disclosure provides compositions. The composition may comprise a protein/peptide of the present disclosure and a pharmaceutically acceptable carrier.

The composition can comprise the proteins/peptides in a pharmaceutically acceptable carrier (e.g., carrier). The carrier can be an aqueous carrier suitable for administration to individuals including humans. The carrier can be sterile. The carrier can be a physiological buffer. Examples of suitable carriers include sucrose, dextrose, saline, and/or a pH buffering element (such as, a buffering element that buffers to, for example, a pH from pH 5 to 9, from pH 6 to 8, (e.g., 6.5)) such as histidine, citrate, or phosphate. Additionally, pharmaceutically acceptable carriers may be determined in part by the particular composition being administered. Accordingly, there are a wide variety of suitable formulations of pharmaceutical compositions of the present disclosure. Additional, non-limiting examples of carriers include solutions, suspensions, and emulsions that are dissolved or suspended in a solvent before use, and the like. The composition may comprise one or more diluents. Examples of diluents, include, but are not limited to distilled water, physiological saline, vegetable oil, alcohol, dimethyl sulfoxide, and the like, and combinations thereof. Compositions may contain stabilizers, solubilizers, suspending agents, emulsifiers, soothing agents, buffers, preservatives, and the like, and combinations thereof. Compositions may be sterilized or prepared by sterile procedure. A composition of the disclosure may also be formulated into a sterile solid preparation, for example, by freeze-drying, and may be used after sterilization or dissolution in sterile injectable water or other sterile diluent(s) immediately before use. Additional examples of pharmaceutically acceptable carriers include, but are not limited to, sugars, such as, for example, lactose, glucose, and sucrose; starches, such as, for example, corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as, for example, peanut oil, cottonseed oil, safflower oil, sesame oil, olive oil, corn oil, and soybean oil; glycols, such as, for example, propylene glycol; polyols, such as, for example glycerin, sorbitol, mannitol, and polyethylene glycol; esters, such as, for example, ethyl oleate and ethyl laurate; agar; buffering agents, such as, for example, magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Additional non-limiting examples of pharmaceutically acceptable carriers can be found in: Remington: The Science and Practice of Pharmacy (2012) 22nd Edition, Philadelphia, PA. Lippincott Williams & Wilkins. For example, a composition comprises a modified peptide, and a sterile, suitable carrier for administration to individuals including humans—such as a physiological buffer such as sucrose, dextrose, saline, pH buffering (such as from pH 5 to 9, from pH 7 to 8, from pH 7.2 to 7.6, (e.g., 7.4)) element such as, for example, histidine, citrate, or phosphate. In various examples, the composition may be suitable for injection. Parenteral administration includes infusions and injections, such as, for example, intramuscular, intravenous, intraarterial, intraperitoneal, subcutaneous administration, and the like.

The compositions may be administered systemically. Compositions may be administered orally, may be administered parenterally, and/or intravenously. Compositions suitable for parenteral, administration may include aqueous and/or non-aqueous carriers and diluents, such as, for example, sterile injection solutions. Sterile injection solutions may contain anti-oxidants, buffers, bacteriostatic agents and solutes, which render the composition isotonic with the blood of the intended recipient. Aqueous and/or non-aqueous sterile suspensions may include suspending agents and thickening agents.

Nasal aerosol and inhalation compositions of the present disclosure may be prepared by any method in the art. Such compositions may include dosing vehicles, such as, for example, saline; preservatives, such as, for example, benzyl alcohol; absorption promoters to enhance bioavailability; fluorocarbons used in the delivery systems (e.g., nebulizers and the like; solubilizing agents; dispersing agents; or a combination thereof).

The compositions of the present disclosure may be administered systemically. The term “systemic” as used herein includes parenteral, topical, oral, spray inhalation, rectal, nasal, and buccal administration. The term “parenteral” as used herein includes subcutaneous, intravenous, intramuscular, intra-articular, intra-synovial, intrasternal, intrathecal, intrahepatic, intralesional, and intracranial administration. Preferably, the compositions are administered orally, intraperitoneally, or intravenously.

Examples of compositions include, but are not limited to, liquid solutions, such as, for example, an effective amount of a compound of the present disclosure suspended in diluents, such as, for example, water, saline or PEG 400. The liquid solutions described above may be sterile solutions. The compositions may comprise, for example, one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers.

In an aspect, the present disclosure provides methods of using proteins/peptides of the present disclosure. The methods may comprise binding Mn^{II} to proteins/peptides of the present disclosure. Following binding, the presence of Mn^{II} can be determined.

In various examples, a method of the present disclosure comprises contacting a protein/peptide of the present disclosure with a sample having or suspected of comprising Mn^{II}. The method may further comprise detecting a signal. The signal may be generated by a change in fluorescence activity due to the proximity of a FRET quencher and FRET donor. The change is fluorescence is indicative for binding of Mn^{II}. In various examples, the protein/peptide having Mn^{II} bound thereto may be separated (e.g., isolated) from the sample to remove some or all of the Mn^{II} from the sample.

Examples of samples include, but are not limited to, water samples (e.g., ponds, rivers), aqueous extracts (e.g., mine tailings and other leachates from mining processes, brines, extracts of battery materials), and biological samples (e.g., samples from a subject, samples from any organism such as a plant, animal, other eukaryote, bacterium). In various examples, the method may be used to isolate Mn^{II} during lithium-ion battery recycling.

In various examples, the protein/peptide is administered to a subject and the protein/peptide binds to Mn^{II}. The binding event may be then detected via fluorescence activity of the FRET pair of the protein/peptide. In various examples, a protein/peptide of the present disclosure binds Mn^{II} with better affinity than other cellular competitors. The method may further comprise detecting a signal. The signal may be generated by a change in fluorescence activity due to the proximity of a FRET acceptor and FRET donor. The change is fluorescence is indicative for binding of Mn^{II}.

In a method of the present disclosure, compositions may be administered by various routes. The compositions of the present disclosure may be administered systemically or orally.

An individual in need of treatment may be a human or non-human mammal. Non-limiting examples of non-human mammals include cows, pigs, mice, rats, rabbits, cats, dogs, other agricultural animal, pet, service animals, and the like.

In an aspect, the present disclosure provides devices. The device comprises one or more proteins of the present disclosures.

Various devices may comprise a protein of the present disclosure. Non-limiting examples of devices include filters, membranes, sensors, handheld detector, plate reader, fluorimeter, biosensors, in-line monitors, and the like. One or more proteins/peptides of the present disclosure may be immobilized onto a surface of the device. Methods for immobilization are known in the art. In various examples, the one or more proteins/peptides are conjugated (e.g., immobilized) onto a resin.

In an aspect, the present disclosure provides kits. The kits may provide one or more proteins of the present disclosure and/or one or more devices of the present disclosure. The kit may include instructions for use of the proteins or devices.

The steps of the method described in the various embodiments and examples disclosed herein are sufficient to carry out the methods of the present invention. Thus, in an embodiment, the method consists essentially of a combination of the steps of the methods disclosed herein. In another embodiment, the method consists of such steps.

The following Statements provide various examples and embodiments of the present disclosure.

Statement 1. A protein capable of binding Mn^{II}, comprising the following sequence: Z¹-MPTTTTKVDIAAFDPDKDGTIHLKDALAAGSAAFDKLDPDKDGTLHAKDLKGRVSE ADLKKLDPDX¹DGTLHKKDYLAAVEAQFKAAX²PDNDGTIX³ARX⁴LASPAGSALVN LIR-X⁵-Z² (SEQ ID NO:1), wherein Z¹ and Z² are optional and are a Forster resonance energy transfer (FRET) pair; X¹ is N or G; X² is N or D; X³ is D or H, X⁴ is E or D, and X⁵ optional and is the peptide sequence GSGC (SEQ ID NO:40), wherein when Z¹ is a FRET donor, Z² is a FRET acceptor and when Z¹ is a FRET acceptor, Z² is a FRET donor, and when X⁵ is present, then Z¹ and Z² are absent, or a protein having at least 75% identity to the residues other than X¹, X², X³, or X⁴.
Statement 2. A protein according to Statement 1, wherein the protein comprises or has the following sequence:

>MnLanM1:
(SEQ ID NO: 2)
MPTTTTKVDIAAFDPDKDGTIHLKDALAAGSAAFDKLDPDKDGTLHAKD
LKGRVSEADLKKLDPDNDGTLHKKDYLAAVEAQFKAANPDNDGTIDARE
LASPAGSALVNLIR;
>MnLanM2:
(SEQ ID NO: 3)
MPTTTTKVDIAAFDPDKDGTIHLKDALAAGSAAFDKLDPDKDGTLHAKD
LKGRVSEADLKKLDPDGDGTLHKKDYLAAVEAQFKAANPDNDGTIDARE
LASPAGSALVNLIR;
>MnLanM3:
(SEQ ID NO: 4)
MPTTTTKVDIAAFDPDKDGTIHLKDALAAGSAAFDKLDPDKDGTLHAKD
LKGRVSEADLKKLDPDGDGTLHKKDYLAAVEAQFKAADPDNDGTIHARD
LASPAGSALVNLIR;
>MnLanM4:
(SEQ ID NO: 5)
MPTTTTKVDIAAFDPDKDGTIHLKDALAAGSAAFDKLDPDKDGTLHAKD
LKGRVSEADLKKLDPDNDGTLHKKDYLAAVEAQFKAADPDNDGTIHARD
LASPAGSALVNLIR;
or
MnLanM4-Cys:
(SEQ ID NO: 30)
MPTTTTKVDIAAFDPDKDGTIHLKDALAAGSAAFDKLDPDKDGTLHAKD
LKGRVSEADLKKLDPDNDGTLHKKDYLAAVEAQFKAADPDNDGTIHARD
LASPAGSALVNLIRGSGC.

Statement 3. A protein according to Statement 1 or Statement 2, wherein the FRET pair is protein/peptide-based.
Statement 4. A protein according to Statement 3, wherein the FRET pair is a yellow fluorescent protein-based and cyan fluorescent protein-based FRET pair.
Statement 5. A protein according to Statement 1, Statemen 3, or Statement 4, wherein Z¹ is a cyan fluorescent protein-based group and Z² is a yellow fluorescent protein-based FRET group.
Statement 6. A protein according to Statement 4 or Statement 5, wherein Z¹ has the following sequence:

(SEQ ID NO: 6)
MVSKGEELFTGVVPILVELDGDVNGHRFSVSGEGEGDATYGKLTLKFIC
TTGKLPVPWPTLVTTLTWGVQCFSRYPDHMKQHDFFKSAMPEGYVQERT
IFFKDDGNYKTRAEVKFEGDTLVNRIELKGIDFKEDGNILGHKLEYNYI
SHNVYITADKQKNGIKAHFKIRHNIEDGSVQLADHYQQNTPIGDGPVLL
PDNHYLSTQSALSKDPNEKRDHMVLLEFVTAAR.

Statement 7. A protein according to Statement 4, Statement 5, or Statement 6, wherein Z² has the following sequence:

(SEQ ID NO: 7)
ELMVSKGEELFTGVVPILVELDGDVNGHKFSVSGEGEGDATYGKLTLKF
ICTTGKLPVPWPTLVTTFGYGLMCFARYPDHMKQHDFFKSAMPEGYVQE
RTIFFKDDGNYKTRAEVKFEGDTLVNRIELKGIDFKEDGNILGHKLEYN
YNSHNVYIMADKQKNGIKVNFKIRHNIEDGSVQLADHYQQNTPIGDGPV
LLPDNHYLSYQSALSKDPNEKRDHMVLLEFVTAAGITLGMDELYK.

Statement 8. A protein according to Statement 1 or Statements 3-7, wherein the protein has the following sequence:

>MnLaMP1:
(SEQ ID NO: 8)
MVSKGEELFTGVVPILVELDGDVNGHRFSVSGEGEGDATYGKLTLKFICTTGKLPVP
WPTLVTTLTWGVQCFSRYPDHMKQHDFFKSAMPEGYVQERTIFFKDDGNYKTRAE
VKFEGDTLVNRIELKGIDFKEDGNILGHKLEYNYISHNVYITADKQKNGIKAHFKIRH
NIEDGSVQLADHYQQNTPIGDGPVLLPDNHYLSTQSALSKDPNEKRDHMVLLEFVTA
ARMPTTTTKVDIAAFDPDKDGTIHLKDALAAGSAAFDKLDPDKDGTLHAKDLKGRV
SEADLKKLDPDNDGTLHKKDYLAAVEAQFKAANPDNDGTIDARELASPAGSALVNL
IRELMVSKGEELFTGVVPILVELDGDVNGHKFSVSGEGEGDATYGKLTLKFICTTGK
LPVPWPTLVTTFGYGLMCFARYPDHMKQHDFFKSAMPEGYVQERTIFFKDDGNYKT
RAEVKFEGDTLVNRIELKGIDFKEDGNILGHKLEYNYNSHNVYIMADKQKNGIKVNF
KIRHNIEDGSVQLADHYQQNTPIGDGPVLLPDNHYLSYQSALSKDPNEKRDHMVLLE
FVTAAGITLGMDELYK;
>MnLaMP2:
(SEQ ID NO: 9)
MVSKGEELFTGVVPILVELDGDVNGHRFSVSGEGEGDATYGKLTLKFICTTGKLPVP
WPTLVTTLTWGVQCFSRYPDHMKQHDFFKSAMPEGYVQERTIFFKDDGNYKTRAE
VKFEGDTLVNRIELKGIDFKEDGNILGHKLEYNYISHNVYITADKQKNGIKAHFKIRH
NIEDGSVQLADHYQQNTPIGDGPVLLPDNHYLSTQSALSKDPNEKRDHMVLLEFVTA
ARMPTTTTKVDIAAFDPDKDGTIHLKDALAAGSAAFDKLDPDKDGTLHAKDLKGRV
SEADLKKLDPDGDGTLHKKDYLAAVEAQFKAANPDNDGTIDARELASPAGSALVNL
IRELMVSKGEELFTGVVPILVELDGDVNGHKFSVSGEGEGDATYGKLTLKFICTTGK
LPVPWPTLVTTFGYGLMCFARYPDHMKQHDFFKSAMPEGYVQERTIFFKDDGNYKT
RAEVKFEGDTLVNRIELKGIDFKEDGNILGHKLEYNYNSHNVYIMADKQKNGIKVNF
KIRHNIEDGSVQLADHYQQNTPIGDGPVLLPDNHYLSYQSALSKDPNEKRDHMVLLE
FVTAAGITLGMDELYK;
>MnLaMP3:
(SEQ ID NO: 10)
MVSKGEELFTGVVPILVELDGDVNGHRFSVSGEGEGDATYGKLTLKFICTTGKLPVP
WPTLVTTLTWGVQCFSRYPDHMKQHDFFKSAMPEGYVQERTIFFKDDGNYKTRAE
VKFEGDTLVNRIELKGIDFKEDGNILGHKLEYNYISHNVYITADKQKNGIKAHFKIRH
NIEDGSVQLADHYQQNTPIGDGPVLLPDNHYLSTQSALSKDPNEKRDHMVLLEFVTA
ARMPTTTTKVDIAAFDPDKDGTIHLKDALAAGSAAFDKLDPDKDGTLHAKDLKGRV
SEADLKKLDPDGDGTLHKKDYLAAVEAQFKAADPDNDGTIHARDLASPAGSALVN
LIRELMVSKGEELFTGVVPILVELDGDVNGHKFSVSGEGEGDATYGKLTLKFICTTG
KLPVPWPTLVTTFGYGLMCFARYPDHMKQHDFFKSAMPEGYVQERTIFFKDDGNY
KTRAEVKFEGDTLVNRIELKGIDFKEDGNILGHKLEYNYNSHNVYIMADKQKNGIKV
NFKIRHNIEDGSVQLADHYQQNTPIGDGPVLLPDNHYLSYQSALSKDPNEKRDHMVL
LEFVTAAGITLGMDELYK;
or
>MnLaMP4:
(SEQ ID NO: 11)
MVSKGEELFTGVVPILVELDGDVNGHRFSVSGEGEGDATYGKLTLKFICTTGKLPVP
WPTLVTTLTWGVQCFSRYPDHMKQHDFFKSAMPEGYVQERTIFFKDDGNYKTRAE
VKFEGDTLVNRIELKGIDFKEDGNILGHKLEYNYISHNVYITADKQKNGIKAHFKIRH
NIEDGSVQLADHYQQNTPIGDGPVLLPDNHYLSTQSALSKDPNEKRDHMVLLEFVTA
ARMPTTTTKVDIAAFDPDKDGTIHLKDALAAGSAAFDKLDPDKDGTLHAKDLKGRV
SEADLKKLDPDNDGTLHKKDYLAAVEAQFKAADPDNDGTIHARDLASPAGSALVN
LIRELMVSKGEELFTGVVPILVELDGDVNGHKFSVSGEGEGDATYGKLTLKFICTTG
KLPVPWPTLVTTFGYGLMCFARYPDHMKQHDFFKSAMPEGYVQERTIFFKDDGNY
KTRAEVKFEGDTLVNRIELKGIDFKEDGNILGHKLEYNYNSHNVYIMADKQKNGIKV
NFKIRHNIEDGSVQLADHYQQNTPIGDGPVLLPDNHYLSYQSALSKDPNEKRDHMVL
LEFVTAAGITLGMDELYK.

Statement 9. A composition comprising a protein according to any one of the preceding Statements and a carrier.
Statement 10. A composition according to Statement 9, wherein the carrier is a pharmaceutically acceptable carrier.
Statement 11. A method for binding and/or detecting Mn^{II} in a sample, comprising: contacting the sample with a protein according to any one of Statements 1 to 8 or a composition according to Statement 9 or Statement 10, and measuring fluorescence activity; and wherein a change in fluorescence is used to determine whether Mn^{II} is bound to the protein. In various embodiments, one or more other metals are present.
Statement 12. A method of Statement 11, wherein the protein has Z¹ and Z² groups.
Statement 13. A method of Statement 11, wherein the protein is immobilized on a substrate.
Statement 14. A method according to Statement 11, wherein the method further comprises separating and isolating the Mn^{II}-bound protein from the sample.
Statement 14a. A method according to Statement 11, wherein the method further comprises eluting the protein using acid or a chelator to purify the Mn(II).
Statement 14b. A method according to Statement 11, wherein the protein is SEQ ID NO:30 and the protein selectively binds Mn^{II} in the presence of one or more other metals and the Mn^{II} is selectively eluted.
Statement 15. A method according to any one of Statements 11 to 14b, wherein the method further comprises imaging.
Statement 16. A method for determining the presence or absence of Mn^{II} in a subject, comprising: administering a protein according to claim 1 to the subject; and measuring fluorescence activity, wherein a change in fluorescence is used to determine whether Mn^{II} is bound to the protein.
Statement 17. A method according to Statement 16, wherein the method further comprises imaging.
Statement 18. A method according to Statement 16 and Statement 17, wherein the subject is a human or non-human.
Statement 19. A device comprising a protein according to any one of Statements 1 to 8.
Statement 20. A kit comprising a protein according to any one of Statements 1 to 8 or a composition according to Statements 9 or 10.

The following examples are presented to illustrate the present disclosure. They are not intended to be limiting in any matter.

### Example 1

This example provides a description of peptides of the present disclosure and uses for same.

The design of selective metal-binding sites is a challenge in both small-molecule and macromolecular chemistry. Selective recognition of manganese(II), the weakest binding of first-row transition metal ions, is particularly difficult. As a result, there is a dearth of chemical biology tools with which to study manganese physiology in live cells, which would advance understanding of photosynthesis, host-pathogen interactions, and neurobiology. Described herein is the engineering of the lanthanide-binding protein, lanmodulin, into the first genetically encoded fluorescent sensors for Mn^{II}, MnLaMP1 and MnLaMP2. These sensors with effective Kₐ(Mn^{II}) of 29 and 7 μM, respectively, defy the Irving-Williams series to selectively detect Mn^{II} in vitro and in vivo. Both sensors enable visualization of kinetics of bacterial labile manganese pools. Biophysical studies indicate the importance of coordinated solvent and hydrophobic interactions in the sensors' selectivity. These results establish lanmodulin as a versatile scaffold for design of novel, selective protein-based biosensors and chelators for metals beyond the f-block.

Described herein is lanmodulin can be re-engineered to sense Mn^{II} with high selectivity over its most important cellular competitors, overcoming the Irving-Williams series. The resulting sensor, MnLaMP1, achieves selectivity through two synergistic mutations in each metal-binding EF hand. These substitutions increase Mn^{II} affinity by >25-fold while suppressing response to other divalent metal ions (e.g., Ca^{II}, Fe^{II}, Zn^{II}) in their physiological concentration regimes. The sensor was used to study manganese homeostasis in Escherichia coli and estimate real-time labile Mn^{II} pool size and dynamics for the first time. Finally, mechanistic analysis of Mn^{II} recognition enables us to design MnLaMP2, a sensor with even higher affinity and selectivity for Mn^{II}.

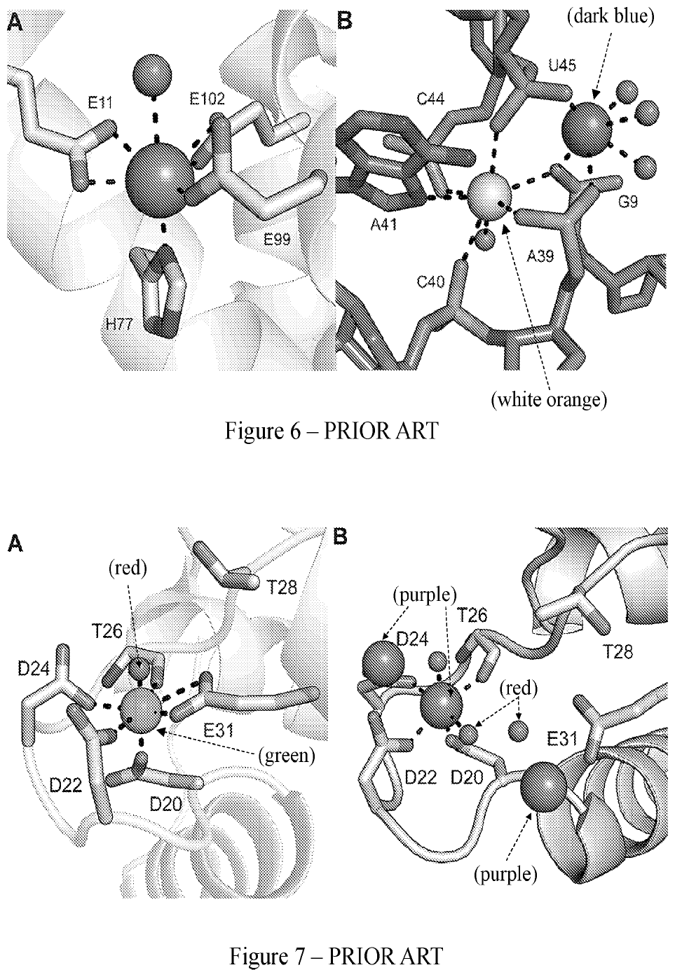

When Nature requires the highest selectivity for Mn^{II} binding, Mn^{II} is recognized with pentagonal bipyramidal coordination geometry, as is evident in metalloregulators: for example, MntR and Mn^{II}-responsive riboswitches (FIG. 6). This is also the preferred geometry of Ca^{II} in EF-hand motifs, for example the canonical Ca^{II} sensing protein, calmodulin, for which structures reveal Ca^{II} coordinated by the residues in the 3^{rd}, 5^{th}, 7^{th}, and 12^{th} positions of the EF hand in the pentagonal plane and the 1^{st} residue and a solvent molecule near the 9^{th} residue in the axial positions (FIG. 7). In fact, some EF-hand proteins display a modest selectivity for Mn^{II} over Ca^{II}; the wild-type LaMP1 sensor undergoes a conformational change in response to 2-fold lower concentrations of Mn^{II} versus Ca^{II} (vide infra, Table 1), and a recent study of an unrelated EF-hand protein, NCS1, shows 3-fold selectivity for Mn^{II} over Ca^{II}. It should be noted that Mn^{II} binding does not always cause the same conformational response in the protein as does Ca^{II}. For example, in calmodulin with Mn^{II} bound, the 12^{th} position Glu residue that is bidentate with Ca^{II} bound is replaced by a single solvent molecule, leading to octahedral geometry and no conformational response, presumably due to the smaller ionic radius of Mn^{II} relative to Ca^{II} (1.04 and 1.20 Å respectively, for the 7-coordinate ions).

Selected constructs in the described evolution of LaMP1's Mn^{II} selectivity are shown in Table 1. The primary metric herein for metal binding selectivity is effective Kₐ (K_{eff}). This value is calculated from the change in the FRET ratio (ratio of emission intensities of YFP and ECFP, F_{529 nm}/F_{478 nm}) during metal titrations. K_{eff} is closely related to the K_{d,app} for the metal-associated conformational response of the protein—for the LanM-based FRET sensor for Ln^{III} ions, LaMP1, the K_{eff} values for the sensor's response to lanthanides are only 2 times the K_{d,app} values for the Ln^{III}-dependent conformational change of LanM itself. The 2-fold difference is an artifact of the choice to use the YFP/ECFP ratio in the calculations, as shown for FRET sensors in general. Whereas metal binding is not necessarily directly coupled to a conformational change, this does seem to be true for the lighter Ln^{III} ions binding to LanM, and data below suggest it is also the case for Mn^{II} binding to LanM variants (vide infra). Therefore, the FRET ratio changes are directly tied to the conformational changes in the Mn^{II}-binding domains, and likely to Mn^{II} binding itself—an important observation for the interpretation of selectivity trends in the sensors of the present disclosure.

Throughout, the number of EF hands mutated is given first, followed by the position of the mutated residue in the EF hand (e.g., D₉ or P₂) and the identity of the resultant residue. Because EF4 has low affinity for Ln^{III} ions, all mutations other than 4P₂A were only made to EF1-3. Substitutions of the axial D9 position to potentially add a softer neutral N donor and better accommodate the smaller Mn^{II} ion (D9Q), or to increase +II selectivity over +III (D₉N) decreased K_{eff} for both Mn^{II} and Ca^{II} but inverted selectivity; however, D₉H decreased K_{eff} and retained slight Mn^{II} selectivity. Importantly, these variants retained a strong fluorescence response and good protein yields. Next, because the E12 residue engages in bidentate coordination in many Ca^{II}-binding EF-hand proteins, its neutralization to Gln was investigated. Surprisingly, the E12Q substitution had only a small effect on K_{eff}. Therefore, it was hypothesized that this residue may bind Mn^{II} via an intermediary water molecule, and the 3E₁₂D variant was investigated to perhaps better accommodate the water. This substitution enhanced K_{eff}, but at the expense of fluorescence response. Reasoning that the 4P₂A variant that responded well to Ca^{II} might also facilitate recognition of the smaller Mn^{II} ion, we also generated several variants in this background; these variants exhibited lower Mn^{II}/Ca^{II} selectivity (Table 3).

TABLE 1
Fluorescence response of LaMP1 variants to Mn^{II} and
Ca^{II}.^{a} Mean (SEM) for three technical replicates. Note that
MnLaMP1 and MnLaMP2 exhibit two-phase responses to Mn^{II}.

| | Mn^{II} | | | Ca^{II} | | |
|---|---|---|---|---|---|---|
| | K_{eff} | | | K_{eff} | | |
| | (μM) | n | F/F₀ | (μM) | n | F/F₀ |
| LaMP1 | 750(10) | 1.9(1) | 4.8 | 1400(100) | 1.1(1) | 3.2 |
| 3D₉N | 300(10) | 1.3(1) | 3.3 | 240(10) | 1.5(1) | 3.5 |
| 3D₉Q | 510(20) | 1.6(1) | 3.0 | 460(10) | 2.5(1) | 3.2 |
| 3D₉H | 280(10) | 1.5(1) | 3.3 | 430(10) | 1.9(1) | 3.3 |
| 3D₉H/3E₁₂Q | 430(20) | 1.3(1) | 2.5 | 710(30) | 1.8(1) | 2.5 |
| 3E₁₂D | 200(20) | 1.1(1) | 1.3 | 310(10) | 1.9(1) | 1.3 |
| 3D₉Q/3E₁₂D | 48(5) | 1.6(3) | 1.5 | 120(10) | 1.8(1) | 1.6 |
| 3D₉H/3E₁₂D | 29(1), | 1.2(1), | 3.0, | 160(10) | 1.3(1) | 3.0 |
| (MnLaMP1) | 100(10) | 0.5(1) | 1.1 | | | |
| 3D₉H/3E₁₂D/ | 7(1), | 1.4(1), | 2.3, | 150(20) | 1.5(2) | 2.7 |
| N87G | | | | | | |
| (MnLaMP2) | 440(10) | 0.8(1) | 1.4 | | | |

^{a}Effective K_{d} (K_{eff}), Hill coefficient (n), and fold response (F/F₀) were calculated from the FRET ratio (F_{527 nm}/F_{478 nm}) in titrations of 0.5 μM sensor in the presence of unbuffered or EGTA-buffered Mn^{II} and Ca^{II} solutions at 25° C., 30 mM MOPS, 100 mM KCl, pH 7.2. Uncertainties are noted in parentheses.

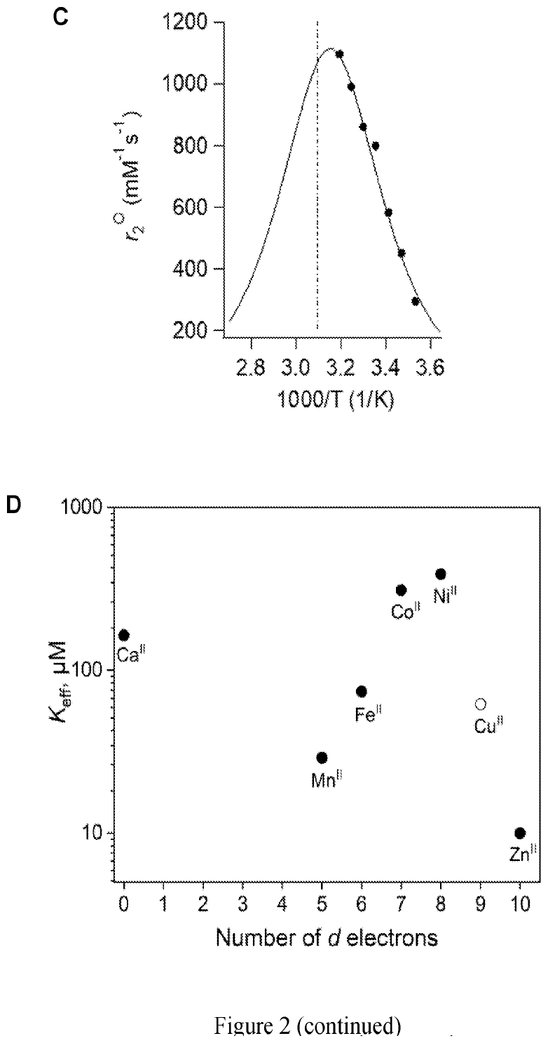

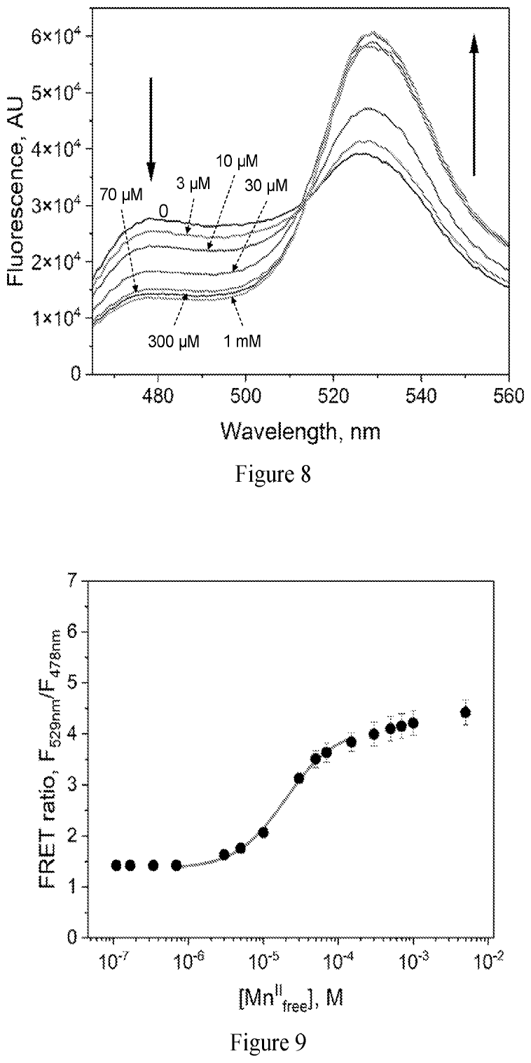

Remarkably, however, combination of the 3E₁₂D mutation with 3D₉H yielded low K_{eff}, >5-fold selectivity over Ca^{II}, and strong (3.3-fold overall) FRET response (FIG. 2B, Table 1, FIG. 8). Two phases were required to fit the Mn^{II} titration data for this variant, with K_{eff}=29 μM (major phase) and 100 μM (minor phase), indicating the involvement of at least 2 EF hands in metal binding. The major-phase K_{eff} value reflects an apparent K_{d} of 15 μM (FIG. 9), similar to the values of 6 and 13 μM reported for Mn^{II}-MntR, suggesting that this sensor can bind and respond to Mn^{II} at physiologically relevant concentrations. This variant was denoted MnLaMP1.

Many of our engineered sensors, including MnLaMP1, exhibit decreased cooperativity, as evidenced by Hill coefficients (n) closer to unity, suggestive of disruption of some of the communication between the EF hands in the wild-type protein. Genetically encoded Ca^{II} sensors derived from engineered EF hands also often lose the cooperativity exhibited by the wild-type Ca^{II}-binding proteins. However, non-cooperativity can, in fact, be beneficial for a fluorescent sensor, because it allows the sensor's response to extend over a larger range of analyte concentrations.

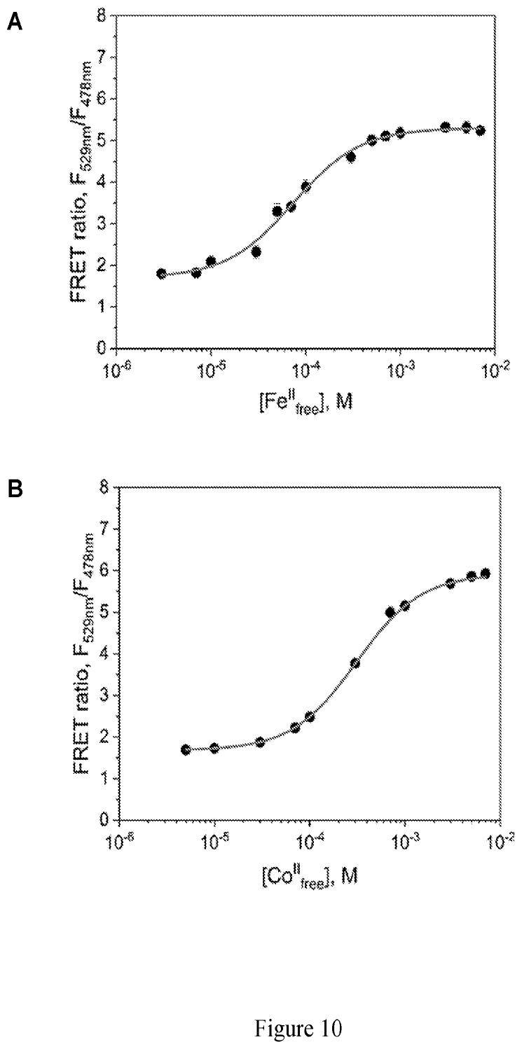

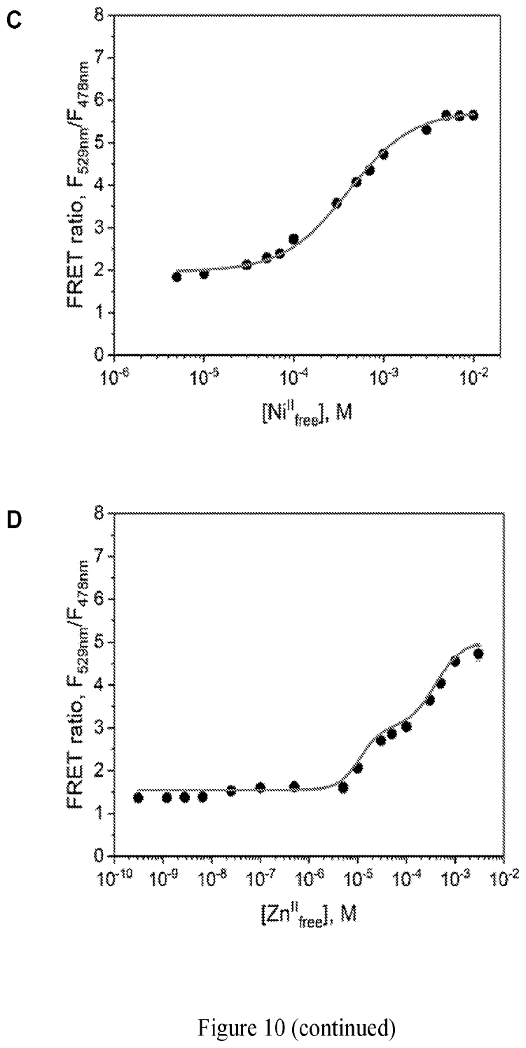

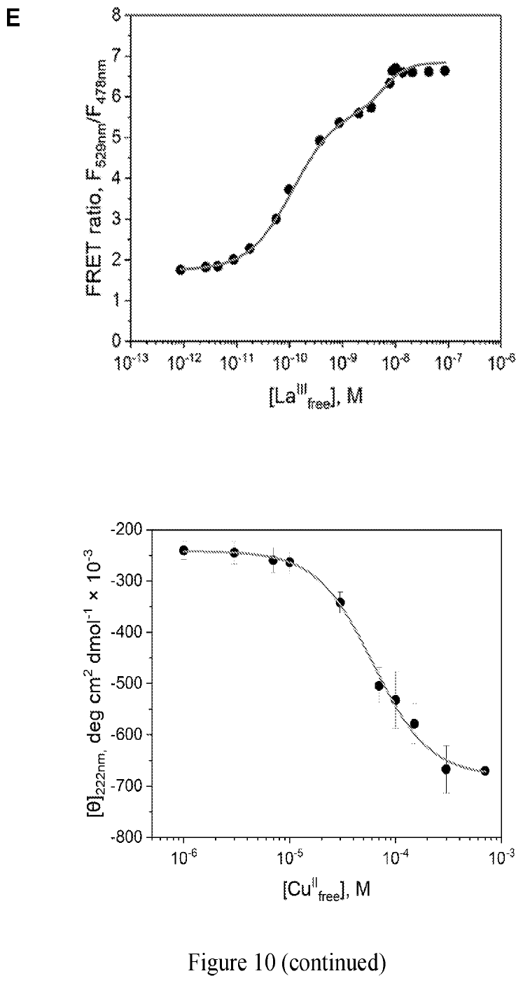

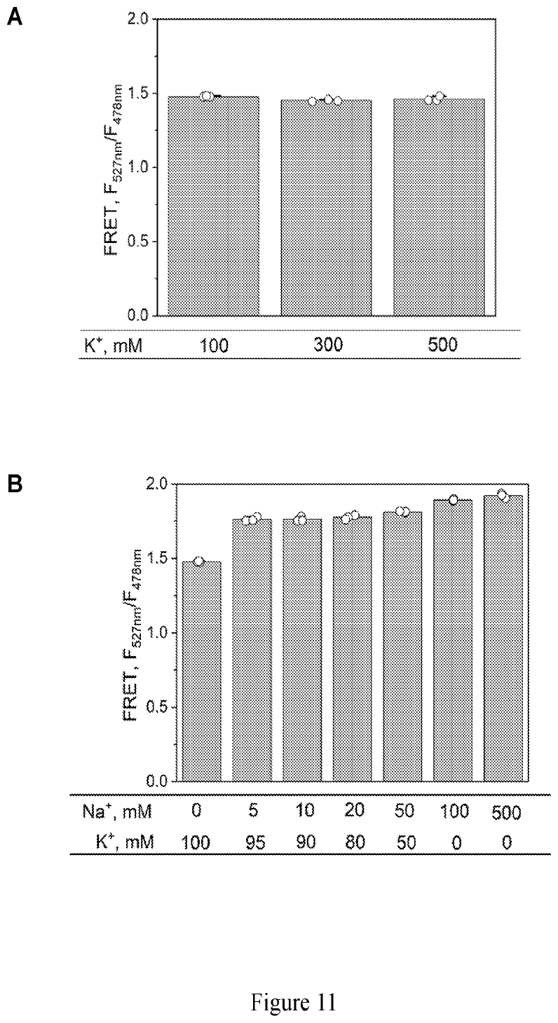

MnLaMP1 selectively responds to Mn^{II} in vitro. Next, the selectivity of MnLaMP1 for Mn^{II} was assessed against other biologically important metal ions. The raw data are shown in FIG. 10 and summarized in Table 2 (selectivity data for other sensors are given in Tables 4 and 5). Interestingly, the divalent metal ions with zero ligand field stabilization energy (LFSE; Ca^{II}, Mn^{II}, and Zn^{II}) exhibit the expected decrease in log K_{eff} with increasing d-electron count, but Fe^{II}, Co^{II}, Ni^{II} deviate in the opposite direction from the expectation from the Irving-Williams series and LFSE trend for an octahedral field (FIG. 2D). (Data for Cu^{II} could not be directly compared owing to low solubility at pH 7.2 and fluorescence quenching, and therefore, response to Cu^{II} was assayed at pH 6.0 (FIG. 10F); these results still suggest a Mn^{II}/Cu^{II} selectivity counter to the Irving-Williams series.) One possible explanation for this surprising result is that MnLaMP1's metal-binding sites favor a non-octahedral geometry in order to optimally induce the protein's conformational response, as intended. Perhaps the most crucial selectivity, and one that has challenged prior sensors, is against Fe^{II}. Labile Fe^{II} concentrations are ˜1 μM in cells; at these levels, MnLaMP1 should not respond to Fe^{II} given its K_{eff} of 74 μM. The sensor maintains >10-fold selectivity over Co^{II} and Ni^{II}, and requires orders of magnitude higher concentrations than those maintained by metalloregulators for these ions. MnLaMP1 exhibits a complex response to Zn^{II} with Hill coefficients>1, unlike the other metals, suggesting that multiple Zn^{II} ions may bind per EF hand (FIG. 10D). Although the first zinc response is slightly tighter than to Mn^{II}, free Zn^{II} concentrations are in the picomolar to nanomolar range in bacteria and eukaryotes, and therefore would be not expected to interfere with Mn^{II} sensing inside the cell. The sensor retains a response to La^{III}, although substantially weaker than that of LaMP1. The important issue of Ca^{II} selectivity is addressed in detail below through in-cell studies. Finally, MnLaMP1 also has 50-fold selectivity over Mg^{II}, it does not respond to K⁺, and displays only a slight (20%) response to Na⁺ below 5 mM (FIG. 11). MnLaMP1's inversion of the Irving-Williams series is especially notable, suggesting that it may uniquely enable in-cell analysis of labile Mn^{II} with little to no interference from other metal ions.

TABLE 2
Fluorescence response of MnLaMP1 and MnLaMP2 (vide infra) to
biologically relevant metal ions.^{a} Mean (SEM) for three
technical replicates. Note that the sensors exhibit two-
phase responses with Mn^{II}, Zn^{II}, and La^{III}

| | MnLaMP1 | | | MnLaMP2 | | |
|---|---|---|---|---|---|---|
| Metal | K_{eff} | | | K_{eff} | | |
| ion | (μM) | n | F/F₀ | (μM) | n | F/F₀ |
| Mn^{II} | 29(1), | 1.2(1), | 3.0, | 7(1), | 1.4(1), | 2.3, |
| | 100(10) | 0.5(1) | 1.1 | 440(10) | 0.8(1) | 1.4 |
| Ca^{II} | 160(10) | 1.3(1) | 3.0 | 150(20) | 1.5(2) | 2.7 |
| Mg^{II} | 1400(200) | 1.4(1) | 3.3 | 1700(170) | 1.1(1) | 2.4 |
| Fe^{II} | 74(6) | 1.2(2) | 2.8 | 78(8) | 1.0(1) | 2.6 |
| Co^{II} | 310(10) | 1.3(1) | 3.2 | 130(20) | 1.0(1) | 2.8 |
| Ni^{II} | 390(20) | 1.0(1) | 3.1 | 380(30) | 0.9(1) | 3.0 |
| Zn^{II} | 10(1), | 2.0(1), | 1.5, | 3(1), | 1.4(3), | 2.3, |
| | 400(10) | 1.6(1) | 2.0 | N.D.^{b} | N.D. | N.D. |
| La^{III} | 0.10(1) | 1.0(1), | 3.7, | 0.07(1) | 0.9(1), | 2.6, |
| | nM, | 1.0(1) | 1.2 | nM, | 3.6(1.4) | 0.8 |
| | 6.3(7) | | | 4.3(5) | | |
| | nM | | | nM | | |

^{a}See Table 1 for conditions. Uncertainties are noted in parentheses.
^{b}N.D.: No saturation observed at 10 mM for second phase, so K_{eff} could not be determined.

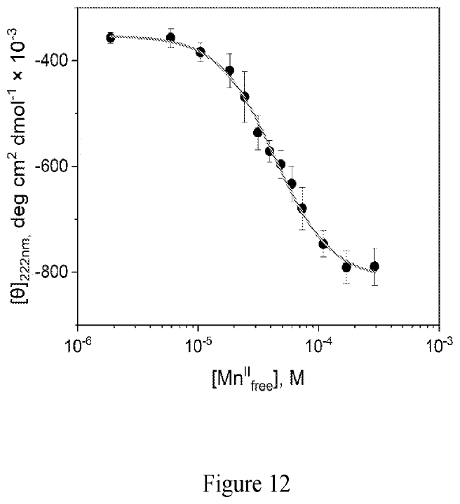

Biophysical characterization of MnLaMP1's Mn^{II}-binding sites. With three metal-binding sites potentially contributing together or independently to response, MnLaMP1 is an unusually complex genetically encoded sensor. Its ˜3-fold FRET response is less than LaMP1's 6-fold response to Ln^{III} ions, indicative of non-identical conformational changes. However, MnLaMP1's FRET change is similar to the 2.9-fold response for Ca^{II} binding to wild-type LaMP1, and the circular dichroism (CD) response of MnLanM (MnLaMP1 with the fluorescent proteins removed) to Mn^{II} is similar in magnitude to that of LanM to Ca^{II} (FIG. 12), perhaps suggesting similar conformational changes in these cases. These considerations led to the examination of which EF hand(s) are responsible for MnLaMP1's response.

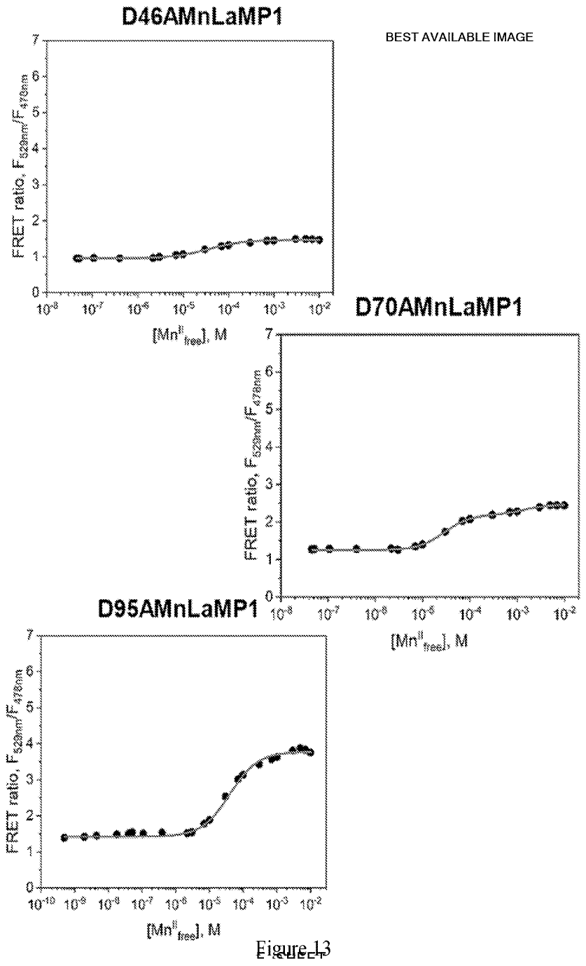

EF hand substitutions. First, individual EF hands were disabled by mutating the D12 residue to Ala and assessed the effects on MnLaMP1's FRET change and K_{eff}. This position was focused on because of the importance of metal coordination by the 12^{th} residue (usually Glu) for conformational responses in EF-hand proteins, as well as the crucial nature of the E12D substitution in generating MnLaMP1. The variants are denoted MnLaMP1-D46A, D70A, and D95A, for substitutions in EF1, EF2, and EF3, respectively. These variants exhibited two-phase responses with similar K_{eff} to MnLaMP1 itself and to each other, ˜30 μM (FIG. 13A, Table 6). However, the variants differed substantially in fold response. The D46A variant reduces the sensor's FRET response to a mere 1.6-fold change. The D70A variant also exhibits a reduced response (1.9-fold for the first phase). However, interfering with EF3 via the D95A substitution (2.7-fold response) had little effect relative to MnLaMP1. Together, these data confirm that Mn^{II} binds to MnLaMP1's EF hands, and they suggest that both EF1 and EF2 are important for MnLaMP1's FRET change, whereas EF3 contributes little. Interestingly, this result contrasts with wild-type LanM's response to Ln^{III} ions, in which EF2 and EF3 are most important and EF1 plays a secondary role. To examine whether these observations were unique to Mn^{II} response, analogous experiments were carried out with Co^{II} (FIG. 13B, Table 6), yielding nearly identical results to Mn^{II}. Therefore, divalent metal ions other than Mn^{II} likely bind to the same sites but generally induce MnLaMP1's conformational change less efficiently than Mn^{II} does.

Isothermal titration calorimetry (ITC). It was sought to corroborate the conclusion that only two EF hands seemed critical to MnLaMP1's function, using ITC studies of MnLanM. Application of this method to characterize Ca^{II} binding to wild-type LanM previously revealed two distinct events, an endothermic phase with stoichiometry of 3, interpreted as Ca^{II} binding to EF hands 1-3, and an exothermic phase interpreted as the protein's overall conformational change. Metal binding may not be propagated to a global conformational change if the local structure at the metal-binding site is incompatible with the protein's optimal folded structure, a hypothesis supported by independent characterization of Ln^{III} ion binding (intrinsic Kₐ) and conformational response (apparent Kₐ) in LanM.

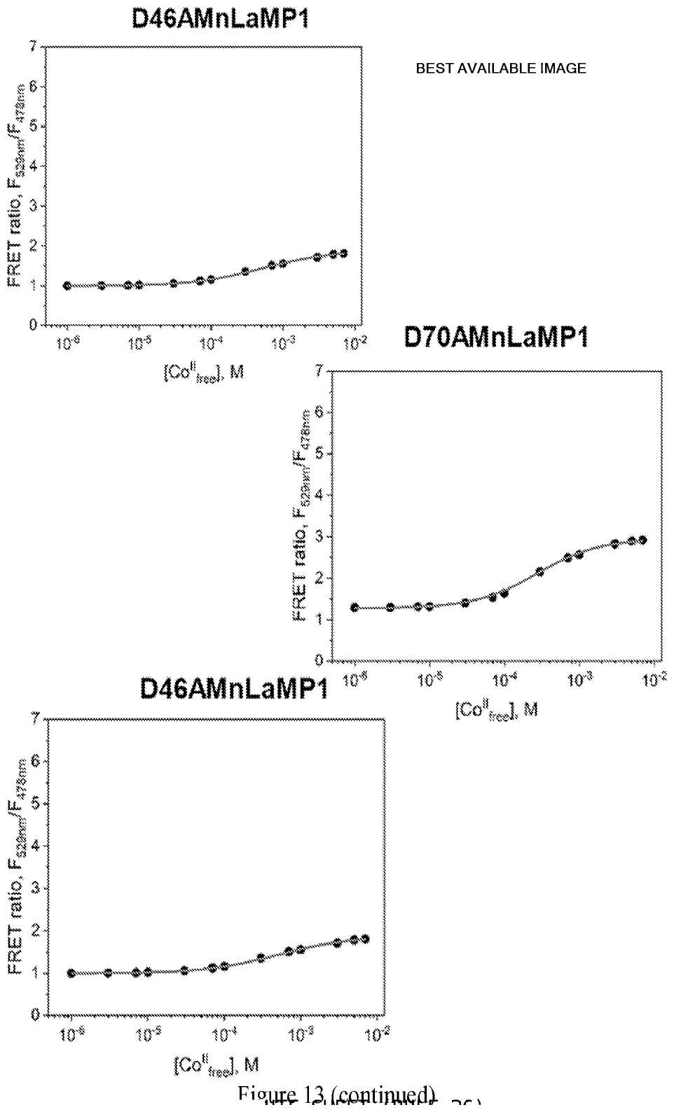

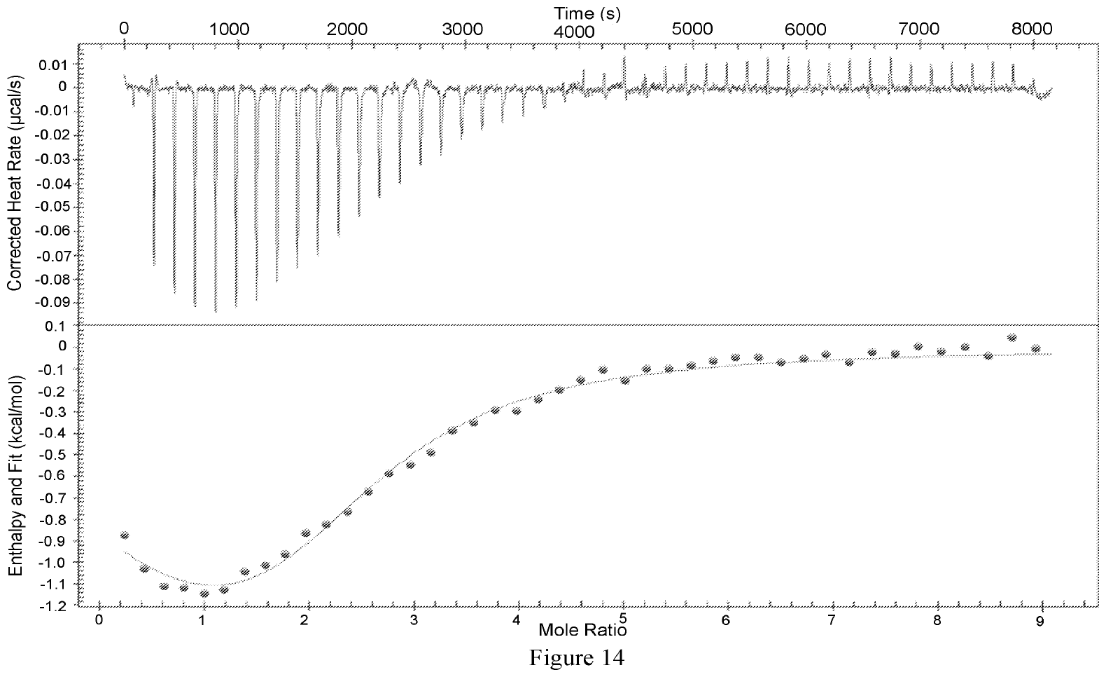

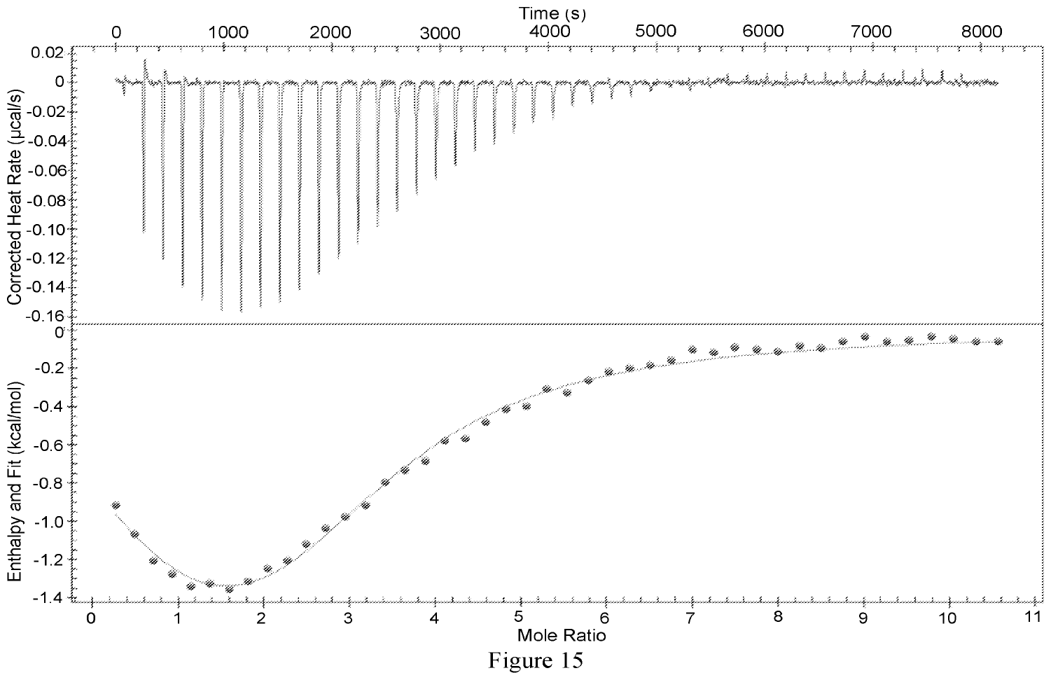

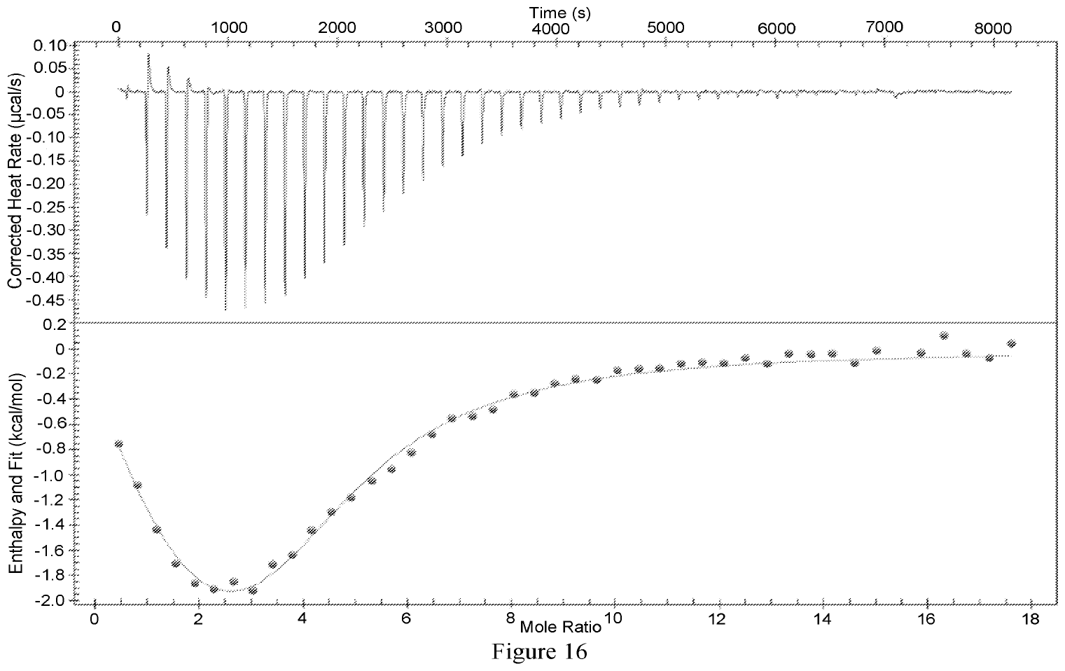

The ITC data for MnLanM (FIGS. 14-16) reveal a similar scenario: an endothermic phase with stoichiometry of ˜1 is combined with an exothermic phase with stoichiometry of ˜2 (Table 7). The Kas for these phases at 25° C. (15 μM) are in good agreement with the K_{eff} values of MnLaMP1 (Table 1, FIG. 9). It was proposed that the endothermic phase represents Mn^{II} binding to one EF hand (perhaps EF3, based on the mutational analyses above) without causing an overall conformational change in the protein, whereas the exothermic phase reflects the conformational change occurring upon metal binding to two other EF hands. Thus, ITC and mutational studies together suggested that two EF hands, likely EF1 and EF2, are most important for MnLaMP1's response.

Solvent coordination probed by ¹⁷ O NMR. Efforts to crystallographically characterize Mn^{II}-bound MnLanM and MnLaMP1 have been unsuccessful to date. Nevertheless, insight can be gained into the coordination environments of the protein-bound Mn^{II} ions (specifically, the presence of Mn^{II}-water co-ligand interactions) by observing the temperature-dependence of MnLanM water-¹⁷O transverse relaxivity

$$( {r}_{2}^{O} ,$$

the paramagnetically induced increase of water-¹⁷O transverse relaxation rate normalized to Mn^{II} concentration in mM) at 11.7 T. In this experiment, the observed

$${r}_{2}^{O}$$

values reflect the number of water co-ligands bound to Mn^{II} (q), the temperature dependence on the mean residency time of the water co-ligand(s) (τₘ), and the strength of the hyperfine coupling interaction between the Mn^{II} electron spin and ¹⁷O nuclei of the exchanging water molecule (Ao/h). The MnLanM

$${r}_{2}^{O}$$

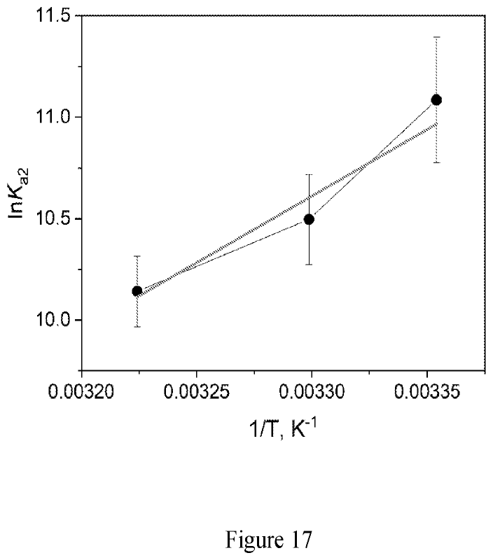

values recorded between 25 and 45° C. are shown in FIG. 2C. This temperature range was used because protein precipitation was observed above 50° C. Based on the ITC-derived thermodynamic parameters (FIG. 17), Mn^{II} is predominantly complexed with MnLanM under our experimental conditions. The temperature-dependent

$${r}_{2}^{O}$$

values for MnLanM were isolated by subtracting the minor contribution of unchelated Mn^{II} (0.14-2.2%, Equation S1, Table 8) to the empirically observed

$${r}_{2}^{O}$$

values.

Within the observed temperature range, MnLaMP

$${r}_{2}^{O}$$

values reside predominantly in the ‘slow exchange’ regime where the time constant for transverse relaxation of coordinated water-¹⁷O (T₂ₘ) is shorter than τₘ. Under the slow exchange condition,

$${r}_{2}^{O}$$

increases with increasing temperature until τₘ=T₂ₘ, at which point

$${r}_{2}^{O}$$

achieves a theoretical maximum. As the temperature continues to increase, the

$${r}_{2}^{O}$$

values move into the ‘fast exchange’ regime where τₘ is now longer than T₂ₘ and

$${r}_{2}^{O}$$

decreases with increasing temperature. When Mn^{II}

$${r}_{2}^{O}$$

values are recorded at sufficiently high field strength, q can be estimated directly from r₂^{O}ₘₐₓ (Equations S2-S8). Although the thermal instability of MnLanM precludes definitive observation of

$${r}_{2 ma x}^{O} ,$$

the temperature dependence is consistent with

$${r}_{2}^{O}$$

approaching

$${r}_{2 max}^{O}$$

consistent with q˜2 near 40° C. The values of q, Ao/h, τₘ and the corresponding enthalpy of activation for water exchange (ΔH^{≠}) were estimated by fitting to temperature dependent

$${r}_{2}^{O}$$

data with Swift-Connick expressions describing two-site exchange. The fits cannot definitively parse individual contributions of q and Ao/h to

$${r}_{2}^{O} ,$$

but based on the relatively narrow range of previously empirically recorded Ao/h values (2.6-4.2×10⁷ rad/s) the fits indicate q between 1.7 to 2.8, τₘ between 39 to 62 ns at 298 K, and ΔH^{≠} between 35 to 41 kJ/mol.

Complexes of wild-type LanM with Gd^{III}, Tb^{III}, and Cm^{III} feature an average q=2. Therefore, MnLanM's Mn^{II} sites have a similar number of waters as Ln^{III}-LanM. This result supports the hypothesis that the point mutations accommodate direct Mn^{II}-water co-ligand interactions. With the caveat that the Mn^{II} coordination may not be identical in each of MnLanM's three metal-binding sites, it is nevertheless remarkable that the protein achieves a low-micromolar K_{d} even with ˜2 coordinated waters per Mn^{II} (compare Mn^{II}-MntR, K_{d}=13 μM with one water, FIG. 6), reinforcing the importance of stabilizing packing interactions unique to the metal-bound state.

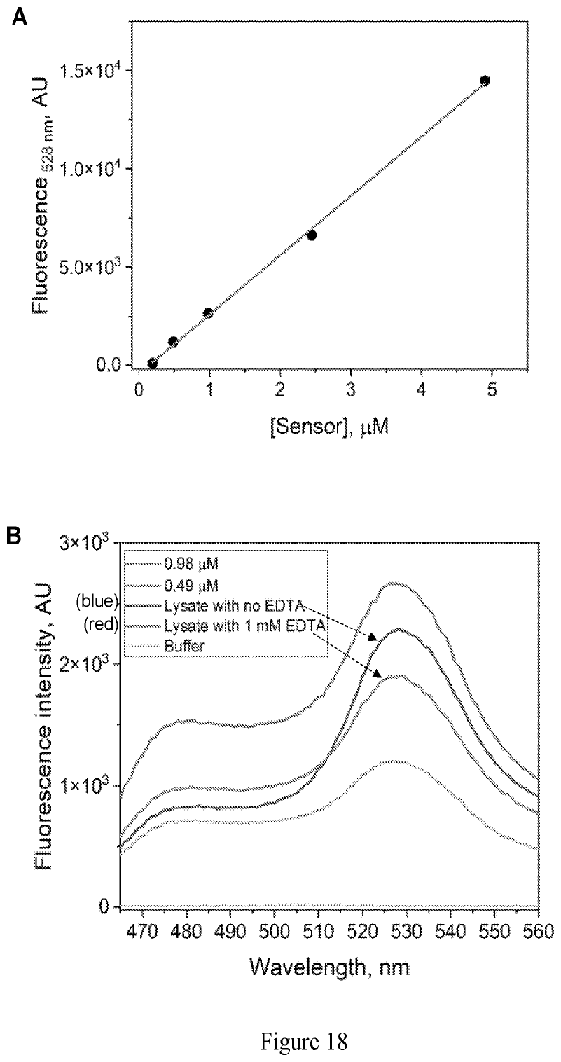

MnLaMP1 selectively senses Mn^{II} in bacteria. It was sought to validate MnLaMP1's function in bacterial cells through constitutive, low-level expression from a tet promoter. First, the average intracellular sensor concentration was determined to be 20±5 μM under our growth conditions (FIG. 18). This value is similar to that of a previously characterized, genetically encoded Zn^{II} sensor from Palmer and co-workers (2-80 μM); because Mn^{II} is thought to be buffered, at least in part, by (poly)phosphates present at concentrations in the tens of millimolar in cells, 20 μM MnLaMP1 is unlikely to significantly perturb free Mn^{II} concentrations.

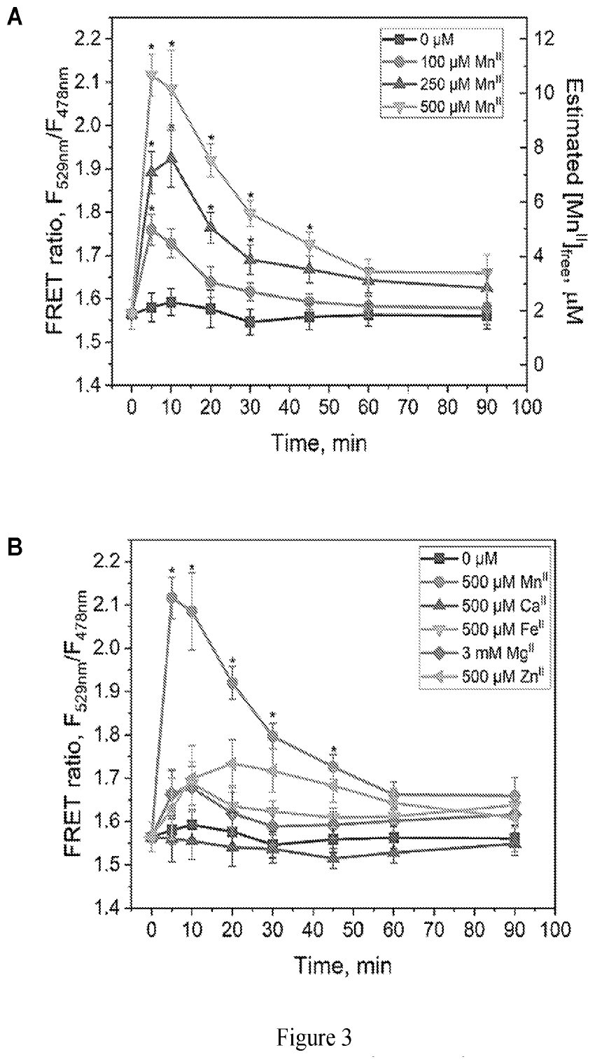

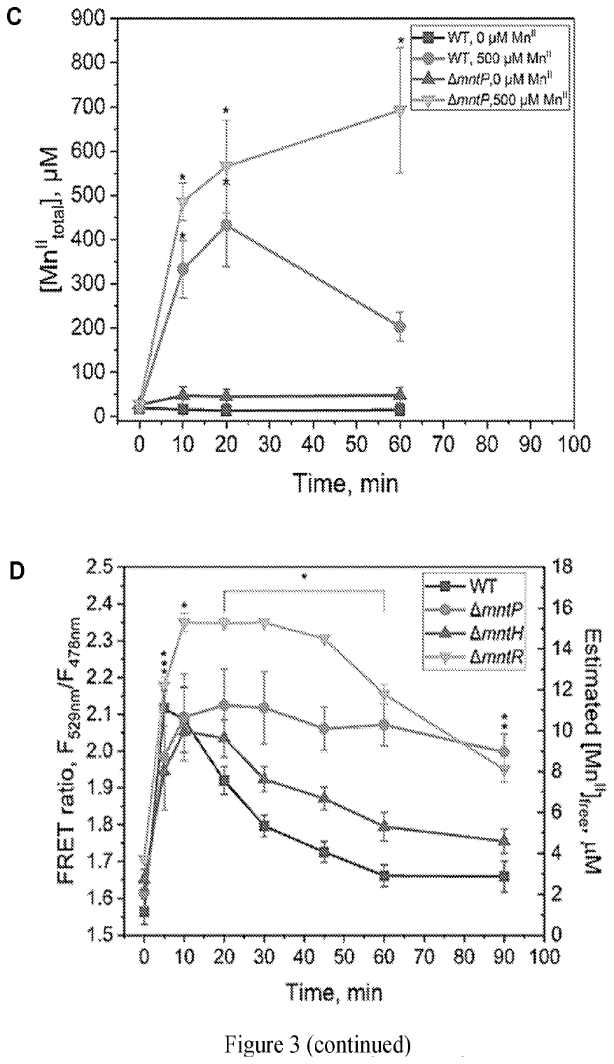

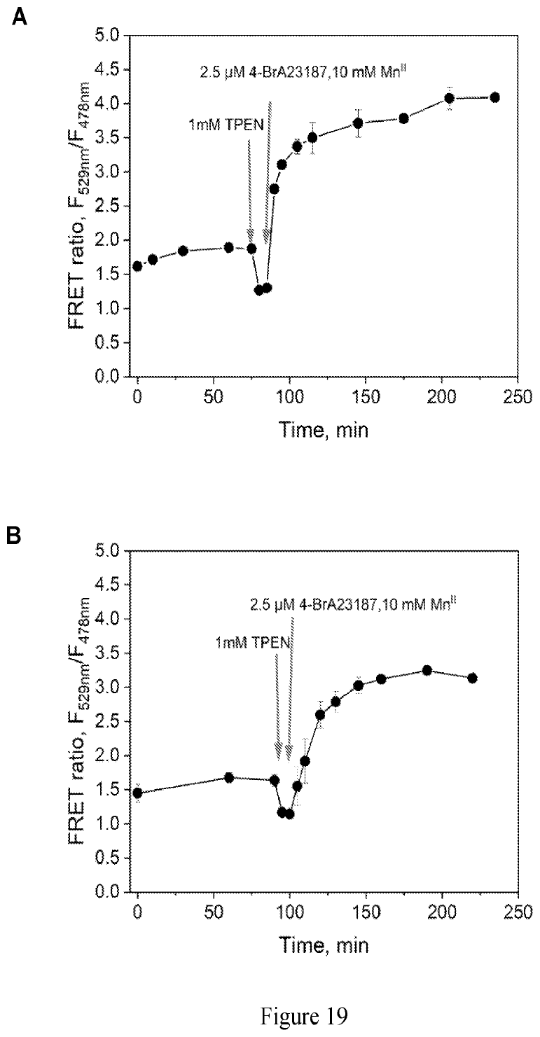

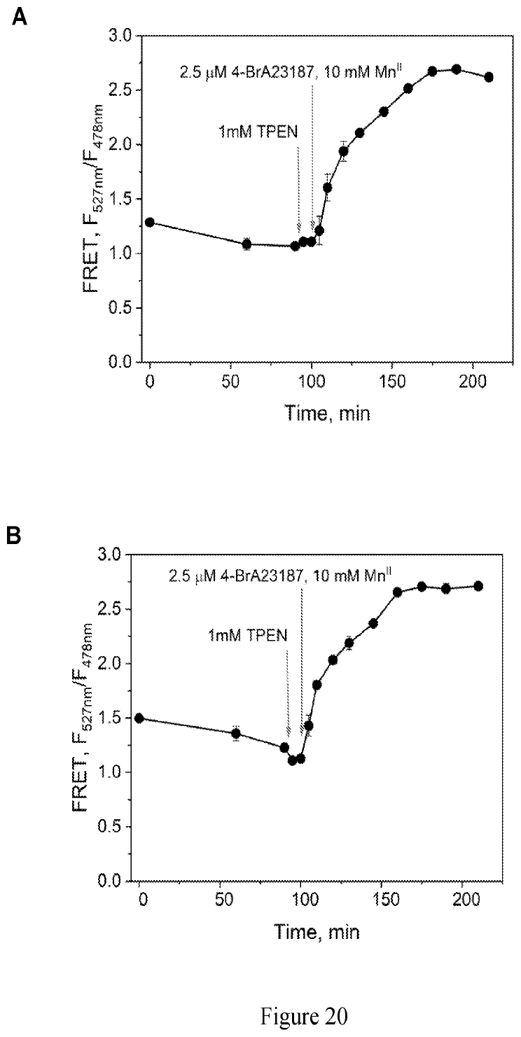

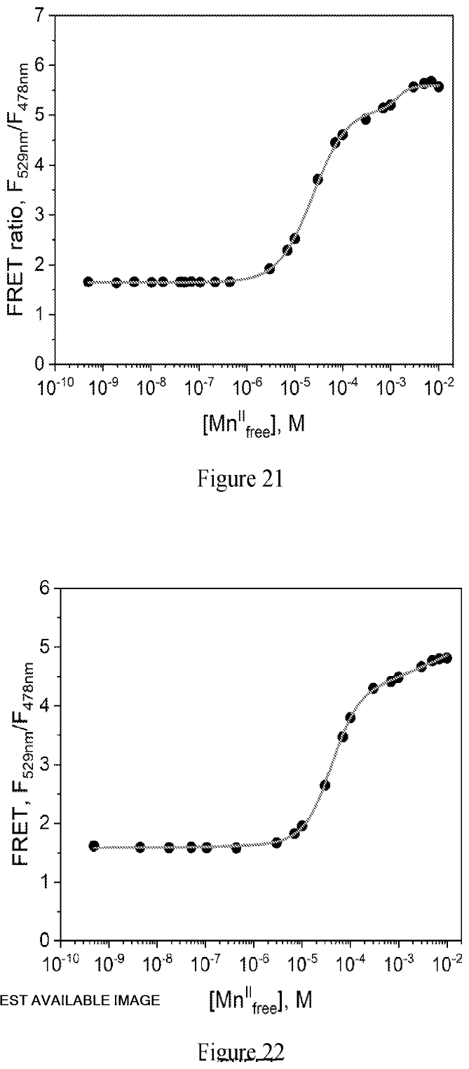

Next, the sensor's in vivo response was calibrated by determining minimum and maximum FRET ratios (Rₘᵢₙ and Rₘₐₓ) by exposing cells to the cell-permeable chelator N,N,N′,N′-tetrakis(2-pyridinylmethyl)-1,2-ethanediamine (TPEN), followed by high Mn^{II} in the presence of the Mn^{II}-selective ionophore 4-BrA23187 (FIG. 19). This approach was an adaptation of standard in-cell sensor calibration methods. The in-cell Rₘᵢₙ and Rₘₐₓ values of 1.3 and 4.2 are similar to those in vitro (1.4 and 4.3); each of these values has an uncertainty of ˜0.1 (FIG. 2B). [Mn^{II}]_{free} cannot be strictly quantified by normalization of in vitro response to the Rₘᵢₙ and Rₘₐₓ ratios, due to small effects of Na⁺ on Rₘᵢₙ (FIG. 20) and of ionic strength on K_{eff} (FIG. 21), and the uncertainty about these concentrations inside the cell. However, because the in-vitro and in-cell Rₘᵢₙ and Rₘₐₓ values correspond closely and the effects of ionic strength and Na⁺ are minor, intracellular [Mn^{II}]_{free} can be reasonably estimated by translating FRET ratios to concentrations using the in-vitro titration (FIG. 2B). Considering the uncertainties in FRET ratio for in vivo and in vitro titrations, a FRET ratio of 1.6 from Rₘᵢₙ and 3.9 can be discriminated from the saturation point, corresponding to a dynamic range of [Mn^{II}]_{free} from 3 μM to 100 μM. Using this method, MnLaMP1's resting FRET ratio inside the cell (1.55, FIG. 3A) is close to the lower limit of this range, suggesting that, at most, [Mn^{II}]_{free}=3 μM under basal conditions. The basal [Mn^{II}]_{free} value compares favorably with the work of Robinson and co-workers in the E. coli relative, Salmonella Typhimurium; using an ex-vivo approach, they calculated that MntR's DNA binding sites are 50% occupied at [Mn^{II}]_{free}=2.6 μM, which serves as an estimate of midpoint buffered Mn^{II} concentration. This result also aligned well with in vitro assays of Bacillus subtilis MntR promoter activity, which estimated resting [Mn^{II}]_{free} at ˜5-10 μM for that organism. The above considerations suggest that ±3 μM is the uncertainty derived from the calibration method for in-cell [Mn^{II}]_{free} quantification, which is larger than the experimental uncertainties from biological replicates in FIG. 3; therefore, the total uncertainty in the [Mn^{II}]_{free} values stated below is estimated to be approximately ±3 μM.

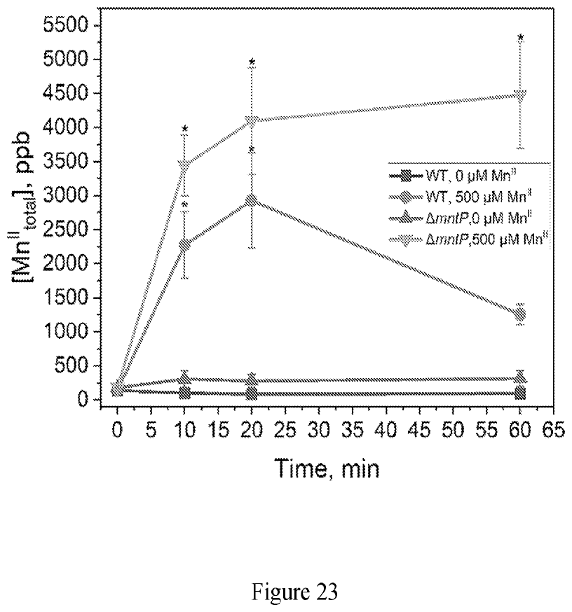

The kinetics of manganese uptake was assessed in wild-type E. coli. Mn^{II} addition (100-500 μM) caused a rapid, dose-dependent increase in FRET ratio within 5-10 min, which dissipated within ˜20-60 min (FIG. 3A). For the 500 μM Mn^{II} condition, the FRET ratio peaked at 2.1, corresponding to [Mn^{II}]_{free}˜10 μM. Parallel ICP-MS studies of total manganese reflected similar kinetics (FIG. 3C, FIG. 23), but with orders-of-magnitude higher manganese concentration than [Mn^{II}]_{free} calculated using MnLaMP1; even under sustained, high levels of extracellular Mn^{II}, 98-99% of cellular manganese is tightly bound to ligands. These studies support the ability of MnLaMP1 to probe labile manganese pool dynamics in bacteria.

Because MnLaMP1 also responds to Mg^{II}, albeit weakly, it may also provide insight into bacterial free Mg^{II} concentrations, about which relatively little information is available. A study using the small-molecule sensor MagFura2 estimated [Mg^{II}]_{free} at 1.0 mM in Salmonella. If Mg^{II} levels are similar in E. coli, a MnLaMP1 resting FRET ratio of ˜3 would be expected; this is not observed, suggesting that intracellular [Mg^{II}]_{free} is significantly lower (≤0.5 mM), more similar to results from eukaryotic cells. This result will be extended below.

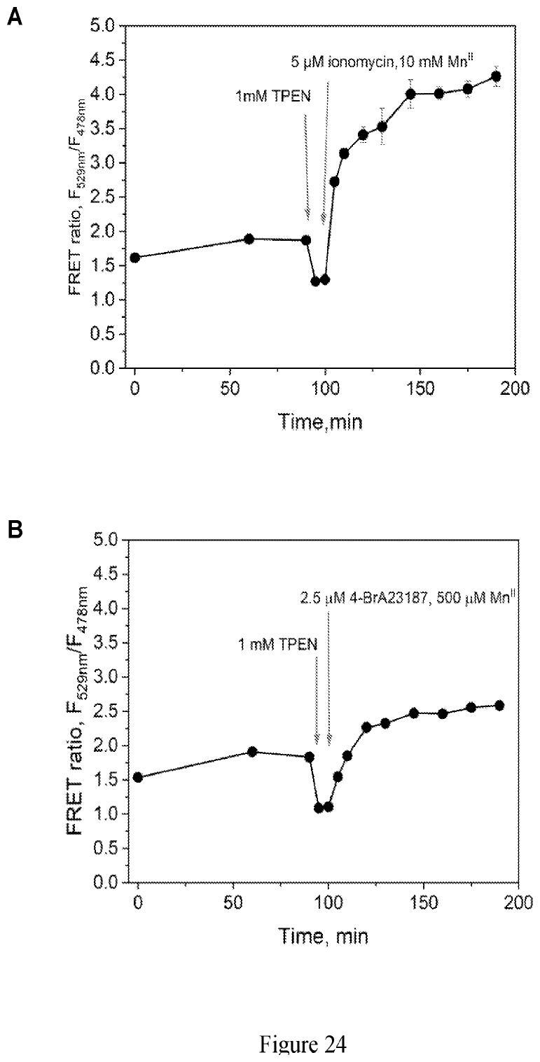

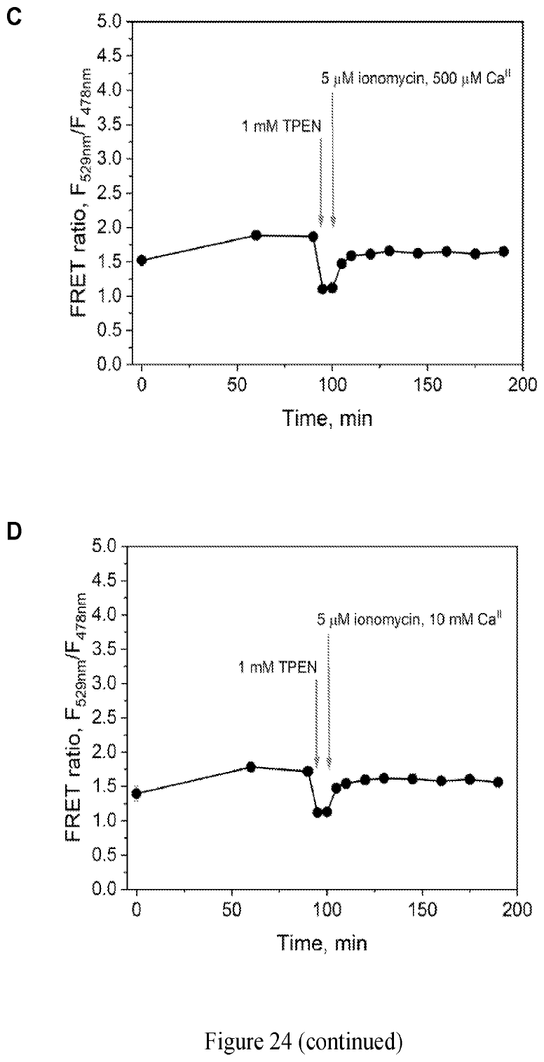

The sensor's in-cell selectivity for Mn^{II} was assessed over major potential interferences, Ca^{II}, Fe^{II}, and Zn^{II} at 500 μM and Mg^{II} at 3 mM (FIG. 3B). Although these metals caused slight initial increases in the FRET ratio (by <0.1, except for 500 μM Zn^{II}, which was slightly greater), none reached statistical significance (p<0.05). It cannot be ruled out that the small response observed with these high concentrations of other metal ions like Zn^{II} might result from displacement of Mn^{II} from its native binding sites followed by binding to the sensor, rather than direct response to the other metal ions. Because free Ca^{II} concentration is maintained at low levels (˜90 nM) in the E. coli cytosol, it is possible that the experiment underestimated the potential for in-cell interference from Ca^{II}. This question is important to address as, in eukaryotic cells, resting cytosolic [Ca^{II}]_{free} is in the sub-micromolar range, but it can transiently reach up to low micromolar concentrations, e.g., 0.5-3 μM. Therefore, E. coli cells were permeabilized with ionomycin, which, and exposed cells to 10 mM extracellular Mn^{II} or Ca^{II} (FIG. 24). Under these conditions, MnLaMP1 is saturated with Mn^{II} ([Mn^{II}]_{free}>100 μM); because ionomycin transports Ca^{II} and Mn^{II} with similar efficiency, intracellular Ca^{II} concentrations should also be similar, and 1-2 orders of magnitude higher than accessed even in a eukaryotic cytosol. However, minimal response was observed, as expected from the in vitro K_{eff} of Ca^{II}-MnLaMP1. Therefore, it was not anticipated that Ca^{II} would interfere with Mn^{II} sensing in eukaryotic cells, with the possible exception of Ca^{II} storage sites such as the endoplasmic reticulum, where [Ca^{II}]_{free} values can be as high as 400 μM. These results demonstrated that the in-vitro K_{eff} values correctly predict MnLaMP1's robust in-vivo selectivity for Mn^{II}, even in the presence of high external concentrations of potential interfering metal ions.

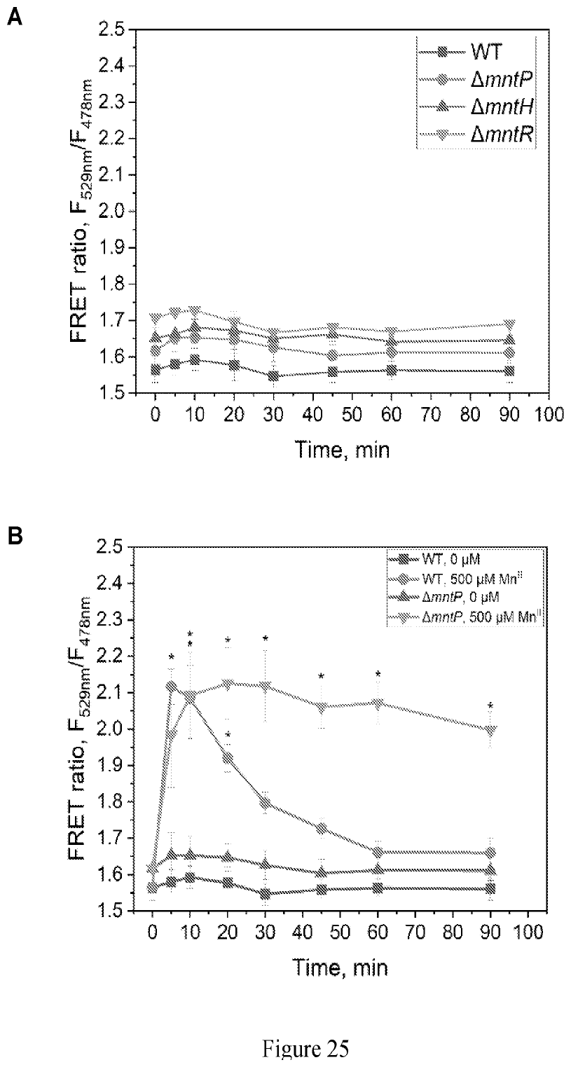

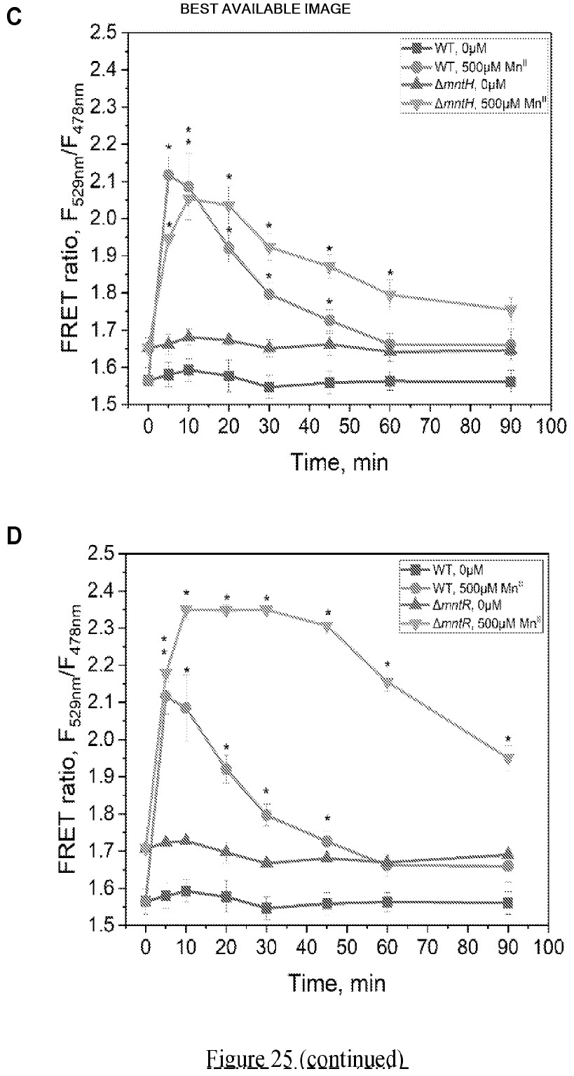

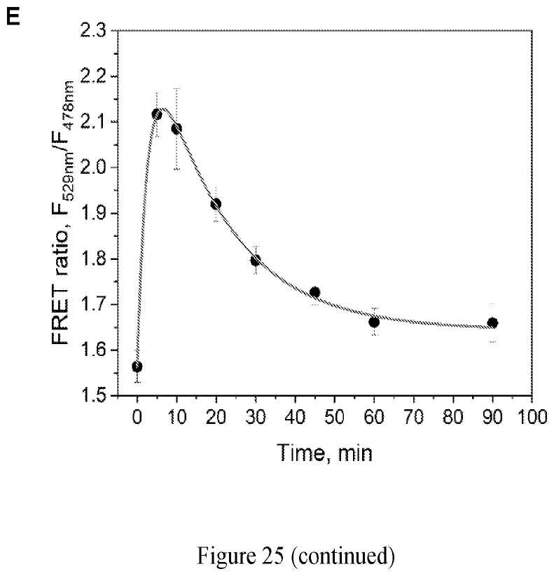

Probing Mn^{II} homeostasis in E. coli. Manganese homeostasis in E. coli is managed by a small set of known players. The Mn^{II}-sensing transcription factor MntR controls expression of the importer MntH, exporter MntP, and small protein MntS. MntP expression is also controlled by a Mn^{II}-responsive riboswitch; a related riboswitch also controls Alx, a protein potentially involved in Mn^{II} import and export. MnLaMP1 was used to investigate the dynamics of labile Mn^{II} pools in strains with MntP, MntH, or MntR disrupted (FIG. 3D, FIG. 25).

Previous studies showed that ΔmntP cells accumulate high levels of total manganese but could not assess how free Mn^{II} is affected. The ICP-MS analysis used herein agrees that the ΔmntP strain accumulates higher total manganese than wild-type, both basally and when grown with 500 μM Mn^{II} (FIG. 3C). Despite higher total Mn levels, [Mn^{II}]_{free}, determined using MnLaMP1, peaks at the same value as WT, ˜10 μM (FIG. 3D). Furthermore, [Mn^{II}]_{free} remains elevated in ΔmntP for the rest of the experiment (90 min). This observation argues that MntP is E. coli's only high-affinity Mn^{II} exporter; in turn, it suggests Alx is either only relevant in the most extreme Mn^{II} stress ([Mn^{II}]_{free}>10 μM) or, perhaps more likely, it is primarily involved in homeostasis of a different metal. Either would explain prior observations that Alx cannot rescue the manganese sensitivity of a ΔmntP strain.

Interestingly, in calibration experiments in ΔmntP, the Rₘₐₓ is lower than in WT, although the Rₘᵢₙ values are similar (FIG. 19B). Although further investigation is warranted, it was proposed that this result reflected an enhanced Mn^{II}-buffering capacity of the ΔmntP cells, preventing sensor saturation even in the presence of high total Mn^{II}. This hypothesis is consistent with the ΔmntP cells taking slightly longer than WT to reach maximum [Mn^{II}]_{free} and the maintenance of that concentration despite higher total Mn than WT (FIG. 3C,D). A higher buffering capacity may compensate for lacking an alternative exporter to efficiently lower [Mn^{II}]_{free}.

In the ΔmntH strain, [Mn^{II}]_{free} increases to the same level as for WT and ΔmntP, but the increase is slower, perhaps because it occurs via a non-specific importer. This result again suggests that 10 μM is a critical [Mn^{II}]_{free} value that the cell is tuned to not exceed. The magnitude of this value is noteworthy, because it corresponds to a predicted ˜90% occupancy of MntR promoter binding sites, such that MntR-mediated repression of MntH and induction of MntP would be nearly maximal. Therefore, [Mn^{II}]_{free} was assessed in the ΔmntR strain. This strain achieves the highest [Mn^{II}]_{free} of all (15 μM), with no decrease until after a 30-min lag. Such high Mn^{II} levels may result from the inability both to repress uptake via MntH and to increase efflux via MntR-mediated upregulation of MntP. Given their shared defect in effluxing Mn^{II}, it is perhaps not surprising that the ΔmntR strain exhibits the same, suppressed Rₘₐₓ value as the ΔmntP strain (FIG. 20). However, in ΔmntR, MntP can also be upregulated via its Mn^{II}-responsive riboswitch. Therefore, these results suggest that the mntP riboswitch does not make a major contribution to Mn^{II} homeostasis unless MntR's response is insufficient, either due to full occupancy of its promoter binding sites-when [Mn^{II}]_{free}>10 μM—or due to mntR deletion. This interpretation also clarifies the disagreement in the literature regarding the Kₐ of the mntP riboswitch, supporting a value in the tens of micromolar.

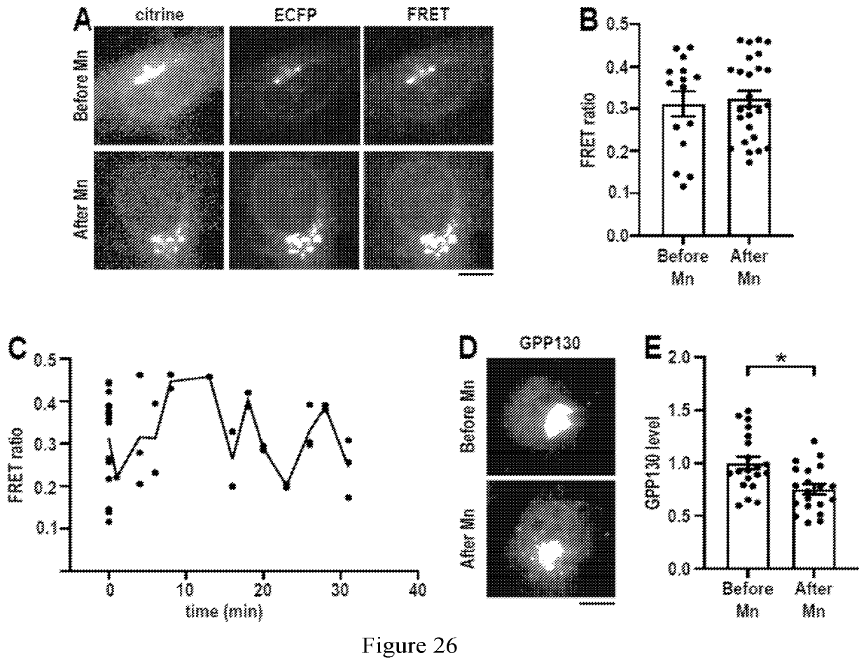

Increasing affinity and selectivity via mutation of a non-coordinating residue. Having demonstrated MnLaMP1's utility in interrogating bacterial physiology, mammalian systems were examined next. Because the Golgi is a major site of manganese accumulation, a Golgi-targeting sequence was appended to MnLaMP1 and expressed it in HeLa cells to investigate whether an increase in Golgi [Mn^{II}]_{free} was observed upon treatment with 500 μM Mn^{II}. While localizing correctly, Golgi-MnLaMP1 did not display a significant, time-dependent response to Mn (FIG. 26). This result suggests that, even in elevated manganese, [Mn^{II}]_{free} is below the detection limit of MnLaMP1 (estimated at 3 μM above); previous work has suggested that [Mn^{II}]_{free} is just below 1 μM in mammalian cells.

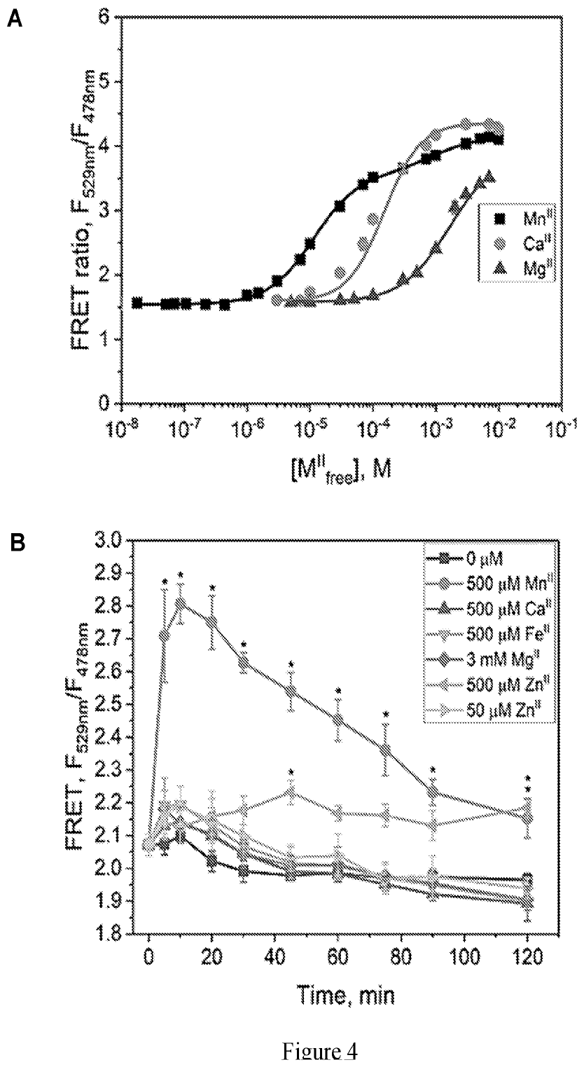

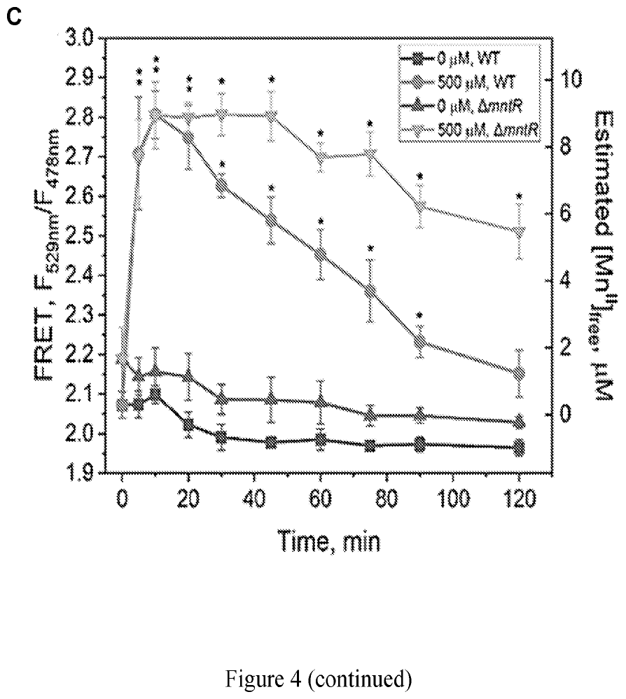

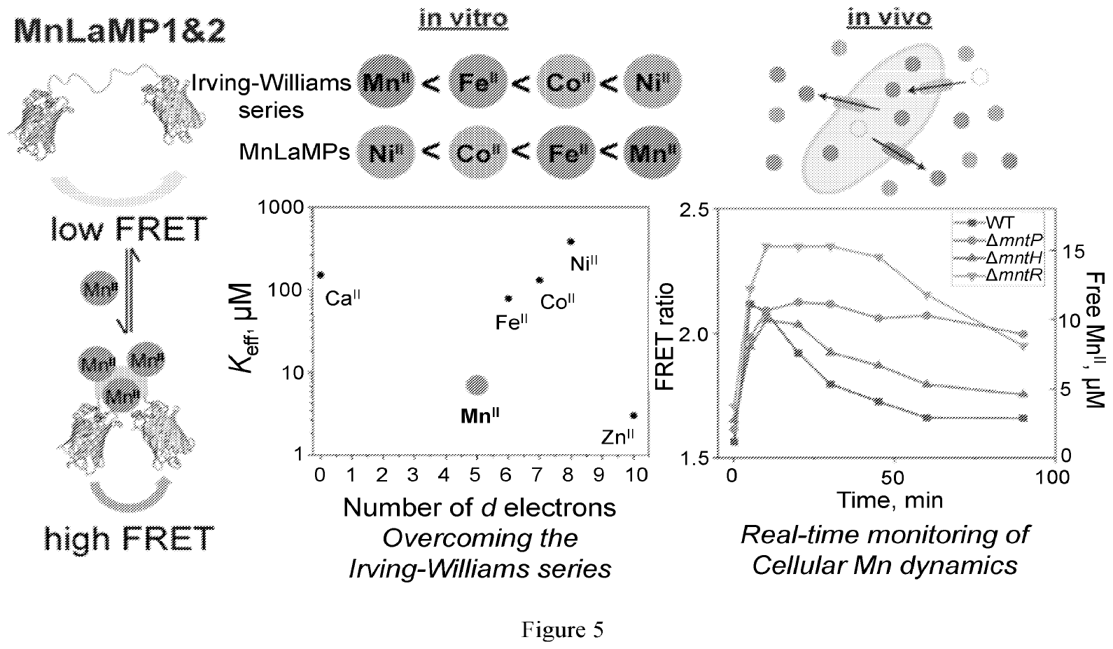

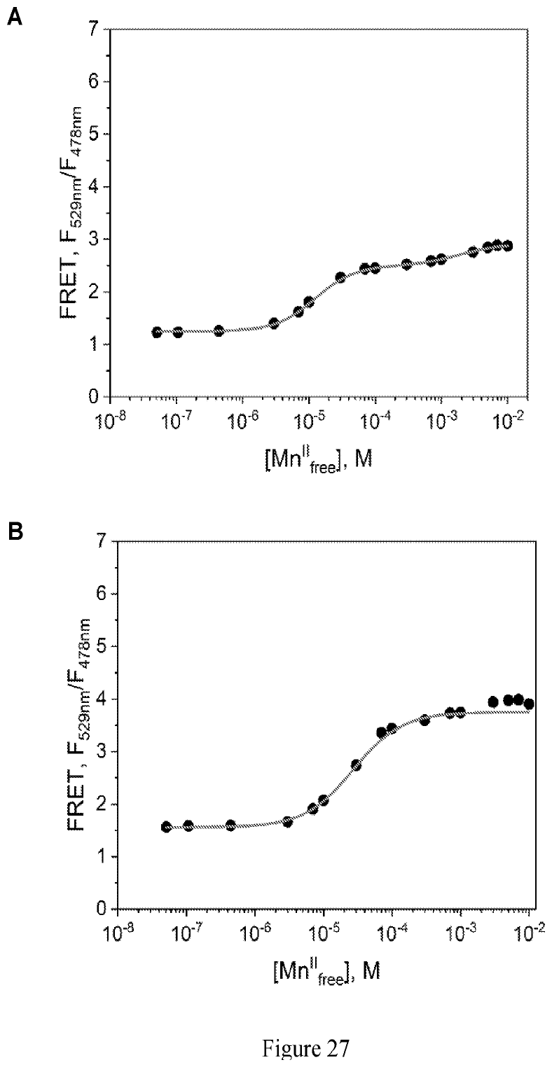

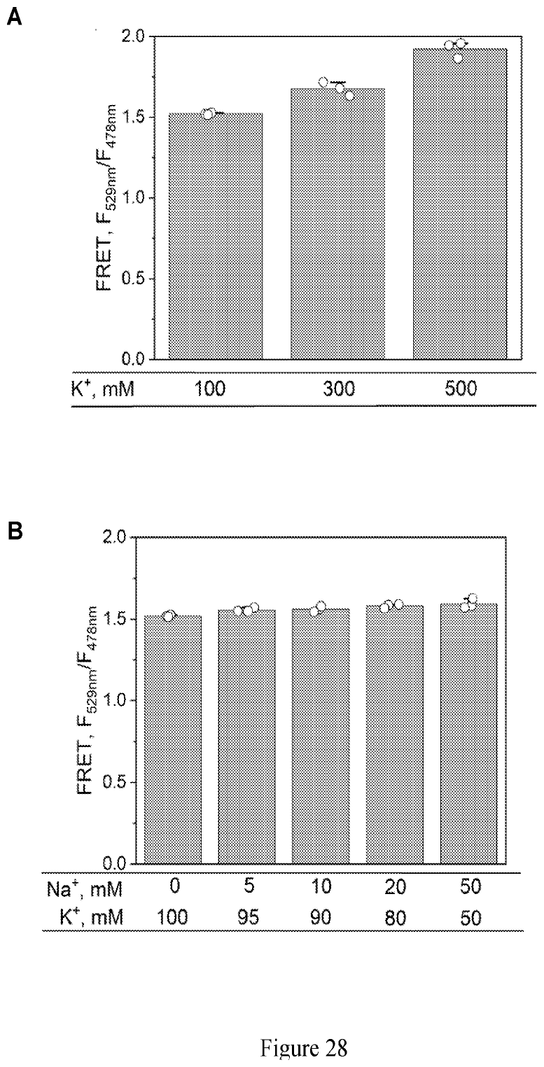

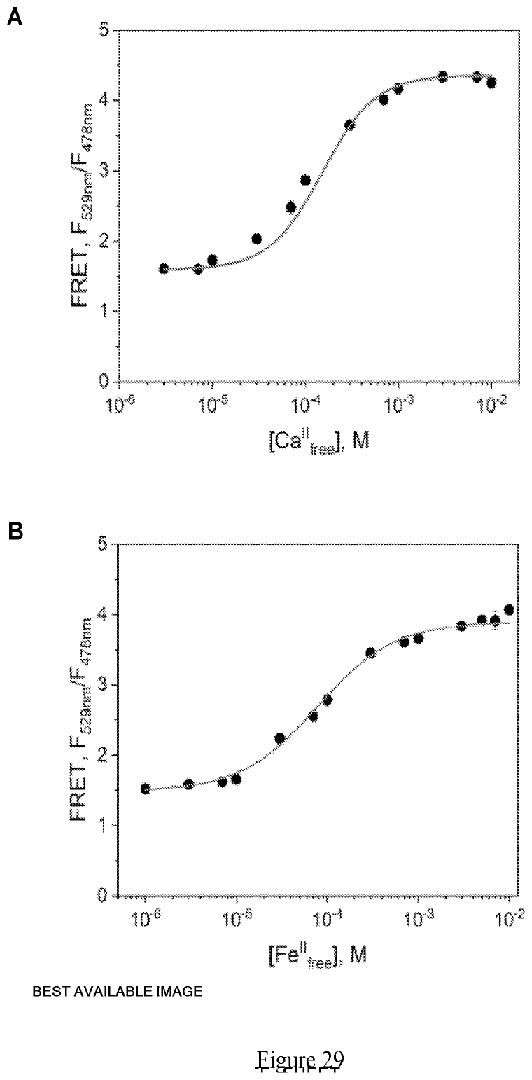

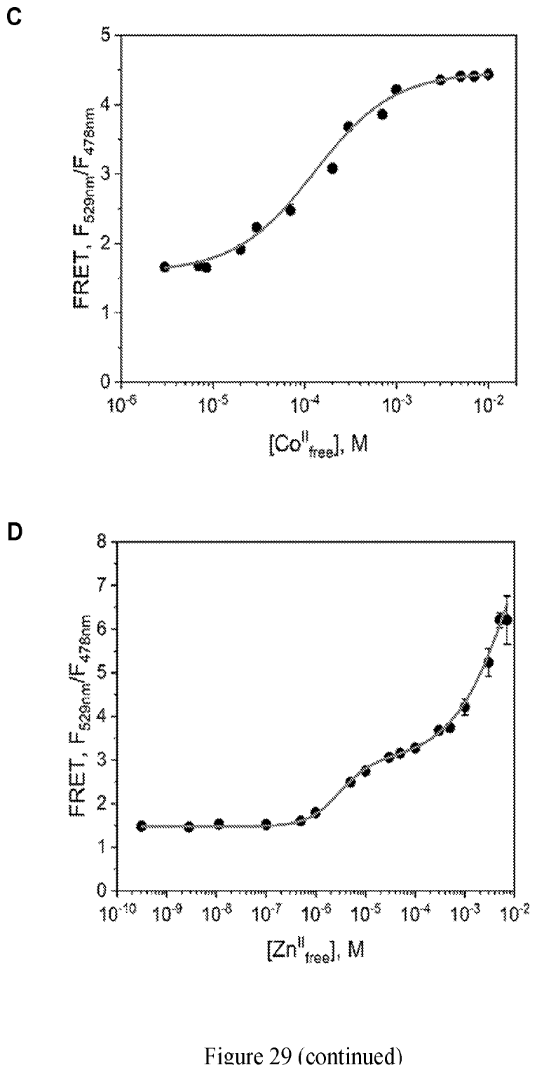

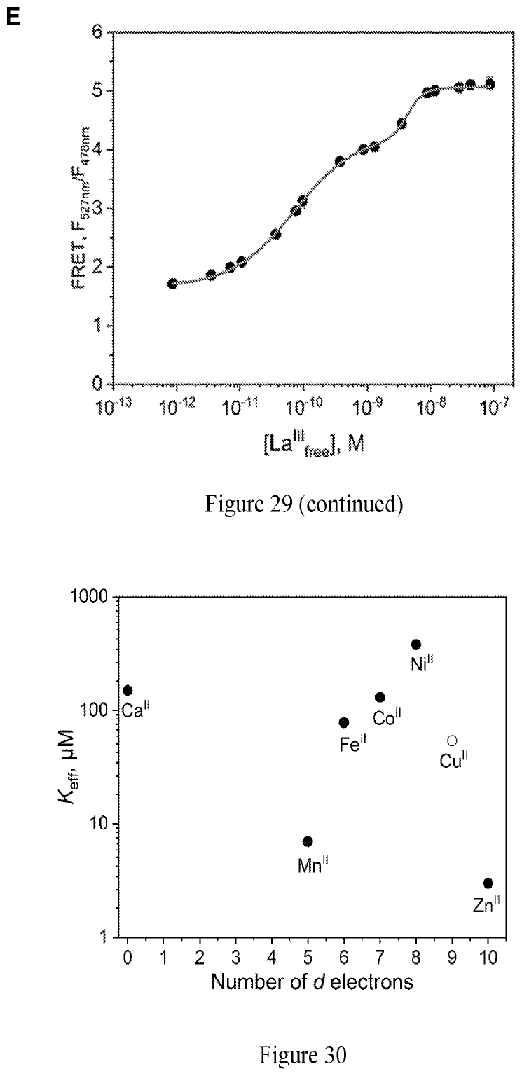

To increase MnLaMP1's Mn^{II} affinity without altering the residues that had already yielded excellent Mn^{II} selectivity, X₄G variants of each of MnLaMP1's functional EF hands were generated. Surprisingly, even though the earlier studies had suggested that EF3 is minimally involved in a productive conformational response, only the Gly variant in that EF hand (N87G; MnLaMP2) decreased K_{eff}, by 4-fold (7 μM, FIG. 4A), with FRET ratio change similar to MnLaMP1 (FIG. 27, Table 9). Thus, it was proposed that the lower K_{eff} of this variant resulted from improved packing interactions that enhance Mn^{II}-EF2 binding, rather than improved metal binding to EF3. Interestingly, this variant also is not sodium-responsive; its slight response to K⁺ would be unlikely to impact cellular studies given typical K⁺ concentrations (FIG. 28). Most importantly, MnLaMP2 responds to Mn^{II} at 4-fold lower levels but the K_{eff} values for the other metals (except Zn^{II}) are largely unchanged (Table 2, FIG. 29), retaining the unusual affinity trend of MnLaMP1 (FIG. 30).

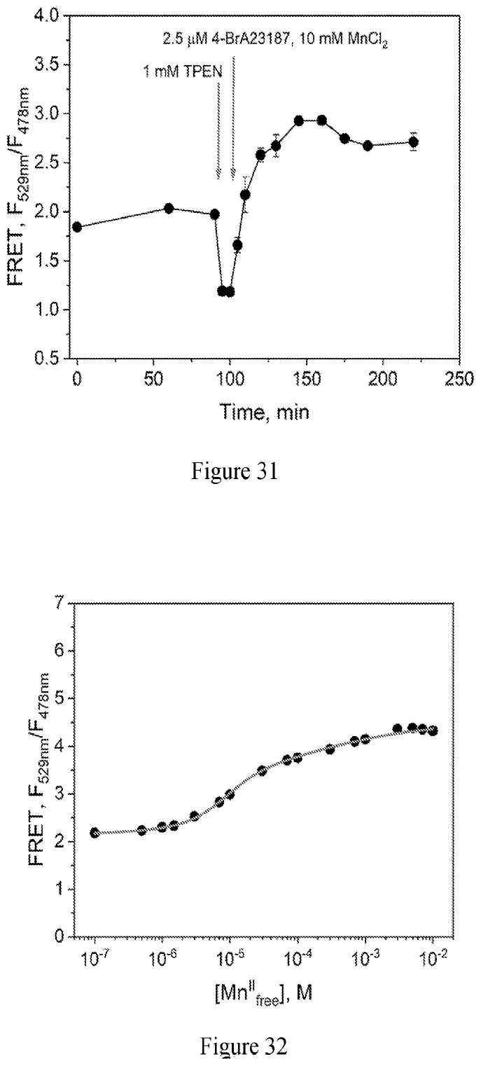

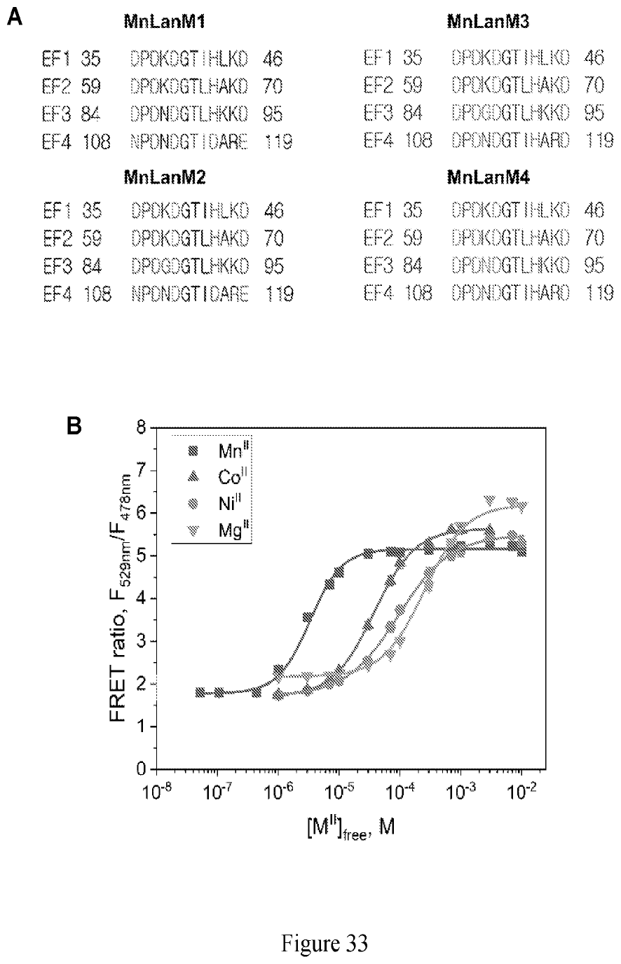

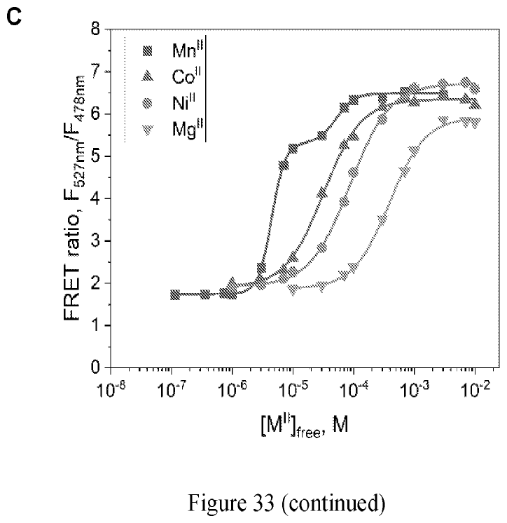

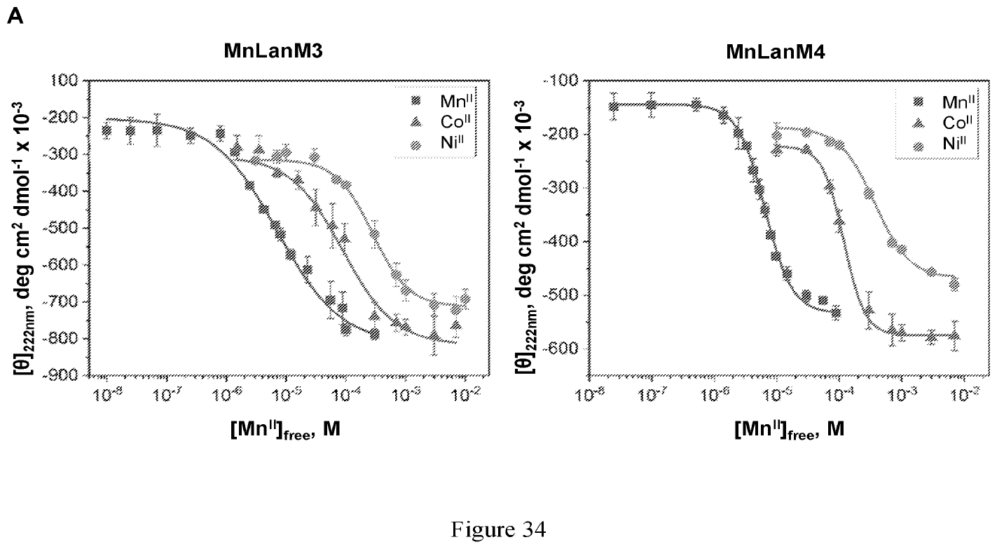

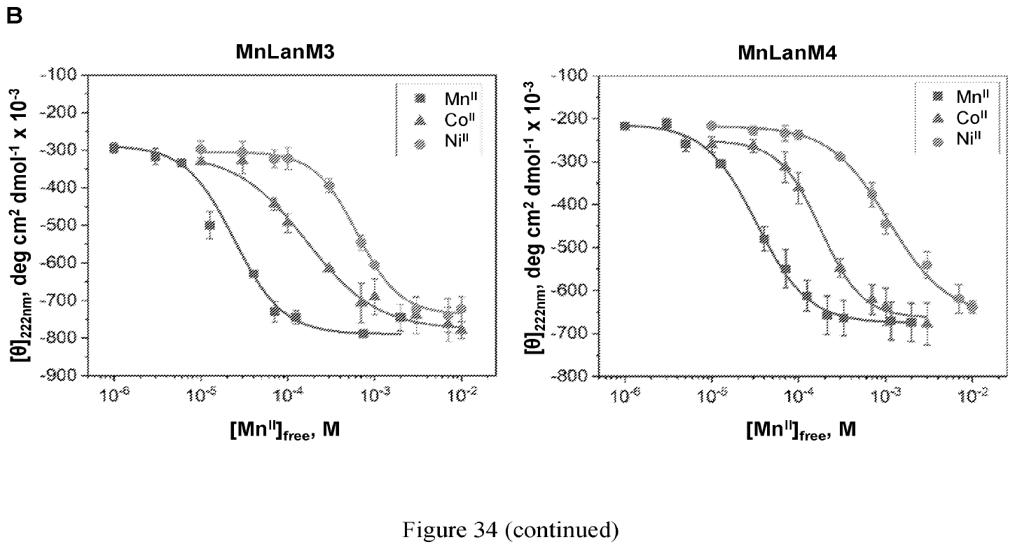

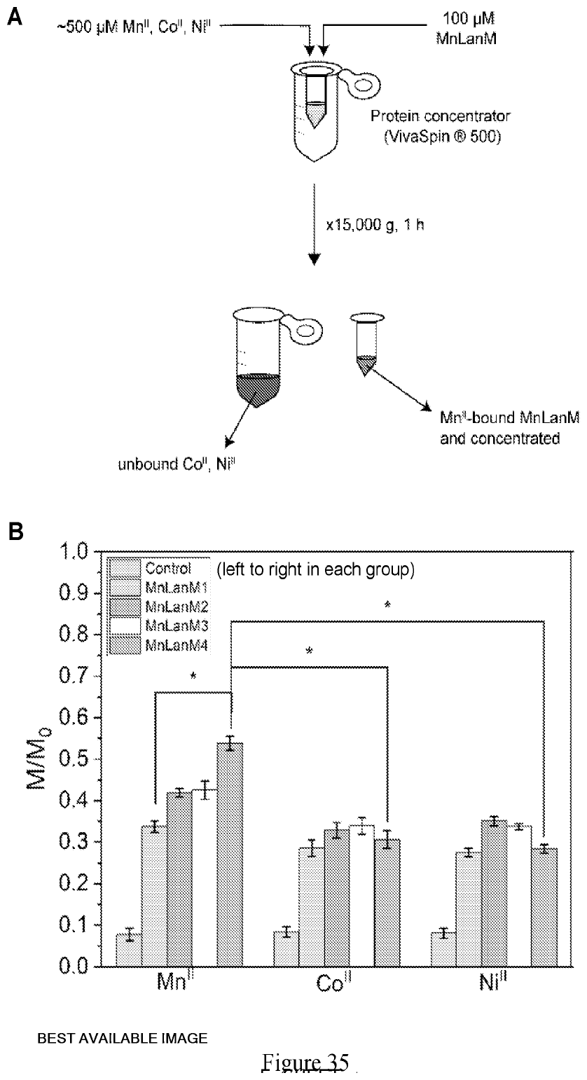

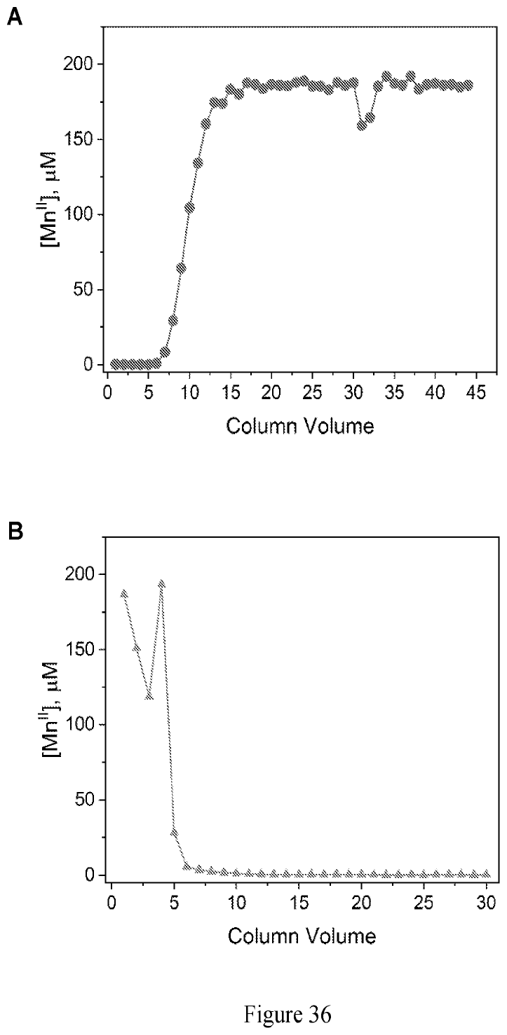

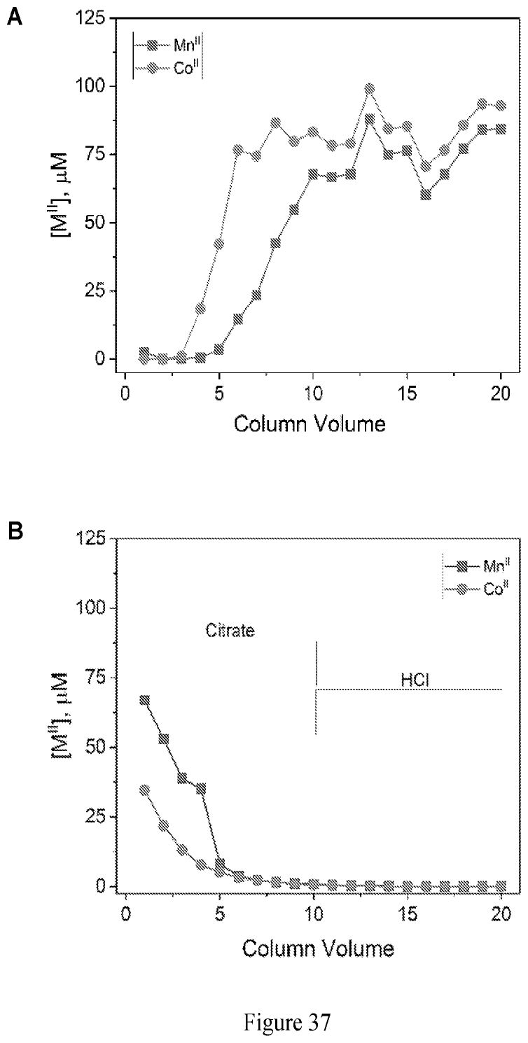

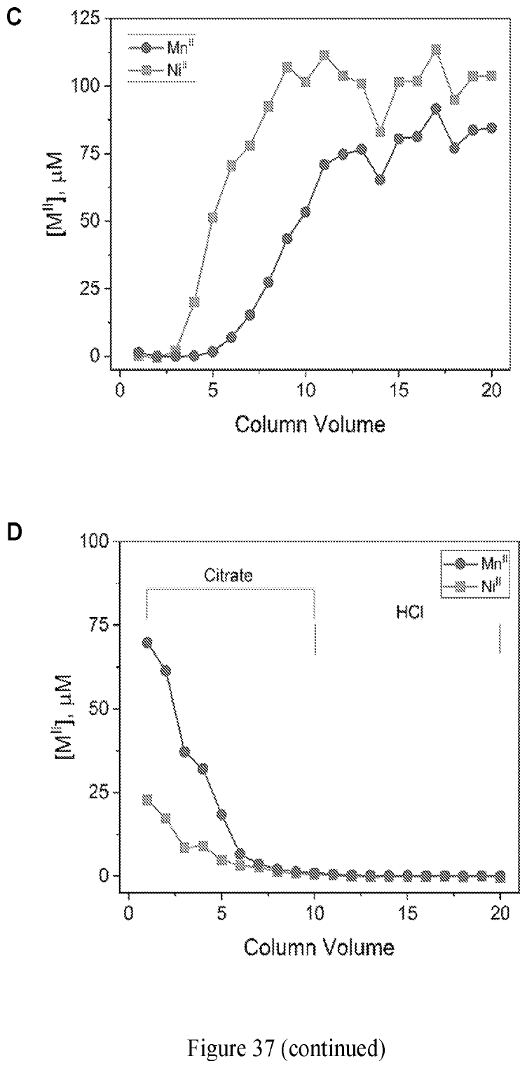

Leveraging this higher in-vitro affinity and selectivity, we characterized MnLaMP2 in E. coli. While the sensor exhibited a resting FRET ratio of 2.0-2.1 in the cell (FIG. 4B), calibration shows that the Rₘᵢₙ is similar to that of MnLaMP1, ˜1.2 (FIG. 31). Intracellular K⁺ levels were not sufficiently high to account for this response, and MnLaMP1 had already suggested that basal [Mn^{II}]_{free}<3 μM. Therefore, the most reasonable explanation for this baseline is response to [Mg^{II}]_{free}, estimated at ˜0.5 mM based on FIG. 4A, supporting the conclusion from MnLaMP1 and lower than previously estimated [˜1 mM or higher]. This unexpected discrepancy, which has important implications for nucleic acid biochemistry, might arise from small-molecule probes forming ternary complexes with Mg^{II} bound to other molecules (e.g., nucleotides), whereas MnLaMP1 likely cannot. Confirmation of this intriguing preliminary result and full assessment of the ability of MnLaMP2 to probe cellular Mg^{II} homeostasis will require further studies.

In-cell calibration studies yield an Rₘₐₓ value of 2.9 (FIG. 31), lower than MnLaMP1 and consistent with only the sensor's first response being saturable in vivo, perhaps because the K_{eff} for the second response is higher than for MnLaMP1. Because of the apparent basal response to Mg^{II} in vivo, a titration with Mn^{II} was used in the presence of 0.5 mM Mg^{II} to convert in-cell FRET ratios to [Mn^{II}]_{free} values; Rₘₐₓ=2.9 corresponds to 10 μM (FIG. 32). This limit is the estimated maximal [Mn^{II}]_{free} in WT E. coli exposed to 500 μM Mn^{II}, based on studies using MnLaMP1 (FIG. 3). Consistent with those results, cellular Mn^{II} uptake assays in WT E. coli monitored using MnLaMP2 (FIG. 4B) reached a FRET ratio of 2.8, or [Mn^{II}]_{free}˜9 μM. The higher [Mn^{II}]_{free} achieved in the ΔmntR strain (15 μM, FIG. 3D) is not apparent in the studies with MnLaMP2 (FIG. 4C) as this concentration is above the saturation point of the in-cell calibration. Thus, MnLaMP2 is best suited to the ranges of [Mn^{II}]_{free} achievable in WT E. coli. As expected from in-vitro characterization, MnLaMP2 not only exhibits higher affinity but also is more selective for Mn^{II} than MnLaMP1 in vivo, exhibiting no response to other metals except 500 μM (but not 50 μM) Zn^{II} (compare FIGS. 3B, 4B). Finally, the consistency in extrapolated [Mn^{II}]_{free} values derived from studies of WT E. coli with MnLaMP1 and MnLaMP2 strongly argues that the calibration method is valid and the sensors provide reliable estimates of cellular free Mn^{II} concentrations.

The present disclosure presents a genetically encoded sensors for Mn^{II}, obtained via rational reprogramming of lanmodulin. These proteins' abilities to defy the Irving-Williams series to favor response to Mn^{II} at physiologically relevant concentrations appear to result from enforcement of non-octahedral coordination environment. The present sensors are ideal for bacterial studies; slightly lower Kas would enable eukaryotic applications. For example, the significant K_{eff} improvement obtained from substitution of a fourth position residue in development of MnLaMP2 suggests that this general strategy can be extended to other non-coordinating EF-hand residues in order to engineer even tighter-binding sensors starting from MnLaMP1 and MnLaMP2.

The experiments in E. coli are in excellent agreement with the predicted setpoints of Mn^{II} homeostatic machinery derived from biochemical and genetic studies in the B. subtilis system, as well as elegant but laborious characterization of Mn^{II}-MntR and MntR-DNA interactions, MntR protein levels, and the number of MntR promoter targets, followed by thermodynamic modeling, by others in the Salmonella system. The free concentration presented herein estimates do rely on the assumption that the sensor reaches quasi-equilibrium with other cellular ligands and that it does not greatly perturb Mn^{II} pools, but the latter is unlikely to be a concern as the average sensor concentration in cells is low, 20 μM. There are also small uncertainties introduced by in-cell calibration, as discussed above. Still, MnLaMP1 enables simple, quantitative evaluation of cellular labile Mn^{II} concentration dynamics in real time. These data help to define the tiered labile concentration regimes in which different regulatory systems become relevant. The cell's exceptional Mn^{II}-buffering capacity is intriguing in that manganese plays a limited role in E. coli physiology; MnLaMP1 may be even better suited for bacteria predicted to maintain higher concentrations of labile Mn^{II}, such as B. subtilis. The observation that Mn^{II}-buffering capacity seems to increase when manganese homeostasis is disrupted may enable identification of the chelators buffering cellular Mn^{II} concentrations in future work. Finally, these results suggest that MnLaMP2 may also provide insight into the long-standing question of labile Mg^{II} levels in bacteria.

General considerations. Chemical reagents were obtained from Thermo-Fisher Scientific, Millipore Sigma, or VWR, unless noted otherwise, at the highest purity available. Primers (Table 10) and gBlocks were ordered from Integrated DNA Technologies (IDT). E. coli strains [5α, and BL21 (DE3)] for cloning and recombinant protein expression, as well as cloning reagents (restriction enzymes, Q5 DNA polymerase, OneTaq DNA polymerase, T4 DNA ligase, KLD reagent and NEBuilder HiFi DNA Assembly Master Mix) were obtained from New England Biolabs. E. coli wild type (BW25113), ΔmntH (JW2388-1) and ΔmntP (JW5830-1) were purchased from the Coli Genetic Stock Center at Yale University; ΔmntR (JW0801-1) was obtained from Horizon Discovery (Table 11). pBAD-D2 was a gift from Amy Palmer and Roger Tsien (Addgene plasmid #37470) and pWCD0941 was a gift from Will C. DeLoache and Michelle C. Chang (see Table 12). Plasmids for expression of D12A variants were custom-ordered from Genewiz. PCR cleanup and miniprep kits were from Omega Bio-tek, and gel extractions used the Zymoclean gel DNA recovery kit from Zymo Research. Vector DNA sequences were confirmed by sequencing at the Huck Genomics Facility and Genewiz.

Materials for protein purification and characterization. Ni-NTA resin was purchased from Thermo Scientific. Protein gel electrophoresis was carried out using Life Tech 16% Tris-glycine gels and a mini gel apparatus. Automated protein chromatography was carried out on a GE Healthcare Biosciences Akta Pure fast protein liquid chromatography (FPLC) system. UV-visible absorption spectra were obtained on an Agilent Cary 60 UV-visible spectrophotometer using a quartz cuvette (Starna Cells). Well plate analyses were carried out using a BioTek Synergy H1 microplate reader or a Tecan Infinite m1000pro microplate reader. Circular dichroism (CD) and isothermal titration calorimetry (ITC) measurements were carried out at the X-ray Crystallography and Automated Biological Calorimetry Facility at Penn State. ICP-MS was carried out at the Penn State Laboratory for Isotopes and Metals in the Environment (LIME). Experiments utilizing Fe^{II} were conducted within a vinyl anaerobic chamber (Coy Lab Products, for ITC measurements) or an MBraun Unilab anaerobic box (all other experiments). The metal salts (≥99% purity unless otherwise indicated) used for in-vitro and in-cell experiments were: CaCl₂)·2H₂O (Sigma), MgCl₂ (VWR), MnCl₂·4H₂O (Sigma), NiCl₂·6H₂O (Sigma), ZnSO₄·7H₂O (Sigma), ammonium Fe(III) citrate (Acros), CoCl₂·6H₂O (Sigma, 98%), CuCl₂·2H₂O (Sigma), ammonium Fe(II) sulfate (Sigma), KCl (Spectrum), NaCl (Fisher), and LaCl₃ (Sigma, ≥99.99%).

Materials for in-cell fluorescence assays. The phosphate-containing MOPS minimal medium (1 L) comprised 100 mL 10×MOPS concentrate (0.4 M MOPS, 0.04 M tricine, 95 mM NH₄Cl, 2.76 mM K₂SO₄, 5 M CaCl₂)·2H₂O, 5.25 mM MgCl₂, 0.5 M NaCl, with pH adjusted to 7.4 using −20 mL of 10 M KOH), 10 mL 0.132 M K₂HPO₄, and ˜890 mL of water. The pH of this medium was adjusted to 7.2 using ˜300 μL of 10 M NaOH and the medium was sterile filtered. Before use, glucose and casamino acids were added to 0.2% final concentration. Final [K⁺] was ˜23 mM, and [Na⁺] was ˜8 mM. Phosphate-free MOPS medium (identical but without phosphate added) was used for in-cell sensor calibration to prevent manganese phosphate precipitation.

Construction of LaMP1 variants. Each LaMP1 variant (Table 12) was constructed using a 352-bp gBlock gene fragment corresponding to the lanM fragment used for construction of LaMP1, with the desired point mutations, and flanked by SphI and SacI sites at the 5′ and 3′ ends. The gBlock was digested using SphI and SacI and purified. pBAD-D2 (Addgene #37470) was similarly digested and, following agarose gel electrophoresis, the vector fragment was purified. The inserts were ligated into the digested vector (1:5, vector:insert) using T4 DNA ligase for 4 h at 23° C. following the manufacturer's protocol, and the ligation product was transformed into E. coli 5alpha cells. Colonies were screened using pBAD-F and pBAD-R and the correct inserts were confirmed by DNA sequencing by Genewiz using primers pBAD-F, ECFP-mid, and pBAD-R.

Expression and purification of LaMP1 variants. LaMP1 variants, including MnLaMP1 and MnLaMP2, were expressed on 200 mL or 1 L scale and purified as known in the arts. Protein concentrations were determined using ε_{515 nm}=77,000 M⁻¹ cm⁻¹. Typical yields were 2-15 mg/L culture, depending on the variant.

Preparation of buffered metal solutions. All buffered metal solutions were prepared in 30 mM MOPS, 100 mM KCl, pH 7.2 (Buffer A). Low- and high-metal buffers for EGTA-buffered Ca^{II} titrations (10 mM EGTA or 10 mM Ca^{II}-EGTA, in Buffer A) and EDDS-buffered La^{III} titrations (10 mM EDDS or 10 mM La^{III}-EDDS, in Buffer A) were prepared as described in the art. For EGTA-buffered Mn^{II} titrations, the “high Mn-EGTA” buffer (10 mM Mn^{II}-EGTA in Buffer A) was prepared in the following manner. In a 50 mL Sarstedt conical tube, 0.0734 g EGTA (99%, 0.19 mmol) was dissolved in 10 mL Milli-Q water. The pH was increased to 8.2 using 1 M KOH. To this solution, 7 μL of 1 M MnCl₂ was added to adjust the pH to 7.2. (KOH must be added prior to Mn^{II} addition to avoid oxidation of the manganese.) The final volume was determined, resulting in 13.3 mM Mn^{II}-EGTA (1.33× stock). To 7.5 mL of this stock, 2.5 mL of Chelex-treated 120 mM MOPS, 400 mM KCl, pH 7.2 was added, and resulting in the final 1× buffer. Calculation of free metal concentrations utilized the K_{d,M} values in Table 13.

In vitro characterization of LaMP1 variants. For unbuffered titrations of LaMP1 variants, the proteins were diluted to 0.56 μM in Buffer A. Each metal stock was prepared as a 10× concentrated solution (30, 70, 100, 300, and 700 μM, and 1, 3, 7, 10, 30, 50, 70, 100 mM). The solutions were mixed—90 μL of the 0.56 μM protein stock and 10 μL of each 10× metal stock—to yield final metal concentrations from 0 to 10 mM. For buffered titrations, LaMP1 variants were diluted to a final concentration of 0.5 μM in the low and high EGTA- or EDDS-buffered metal solutions, which were mixed in various ratios to yield the final metal concentration ranges shown in Table 14. Each sample was prepared in Greiner Cellstar 96-well half-area μClear plates. Fluorescence intensity was measured by emission from 460 nm to 560 nm with 433 nm excitation and 1-nm steps using a BioTek Synergy H1 microplate reader with a gain of 89. FRET ratios were determined from the fluorescent emission ratio F_{529 nm}/F_{478 nm}, where F_{478 nm} is the average ECFP emission over 476-480 nm and F_{529 nm} is the average citrine emission over 526-530 nm. Experiments were carried out at 25° C. For anaerobic titrations with Fe^{II}, protein samples and buffers were deoxygenated on a Schlenk line and brought into a glovebox, along with 96-well plates and ferrous ammonium sulfate. Samples were prepared in the 96-well plate, which was sealed in 96-well sealing tape (Thermo Fisher), and the plate was removed from the anaerobic chamber and fluorescence intensities were measured under same conditions as other metal titrations.

Calculation of free metal concentrations for K_{d} determination. Free metal concentrations in chelator-buffered metal titrations using EGTA, EDDS, and citric acid were determined as known in the art using the stability constants tabulated and the values for K_{d,M}, the effective K_{d} of the ligand for metal under the experimental conditions, which are given in Table 13.

Construction of untagged MnLanM. pET24a was digested by NdeI and EcoRI for 1 h at 37° C. Following gel electrophoresis (1% agarose), the digested vector was excised and purified. The LanM domain (MnLanM) was amplified from pBAD-MnLaMP1 using primers NdeI-MnLanM-F and EcoRI-MnLanM-R (Table 10) and the purified PCR product was digested using NdeI and EcoRI for 1 h at 37° C. and purified. The insert and vector were ligated at 5:1 insert:vector ratio using T4 DNA ligase for 4 h at room temperature following the manufacturer's protocol. The ligation products were transformed into 5a cells, plated on LB-agar containing kanamycin (Km) at 50 g/mL and the transformants were screened using T7P and T7T, and correct plasmids were confirmed by sequencing.

Expression and purification of untagged MnLanM. MnLanM was expressed on 2 L scale and purified. Protein concentration was determined using ε_{275 nm}=1400 M⁻¹cm⁻¹. Protein yield was ˜16 mg/L culture.

Isothermal titration calorimetry (ITC). Binding of Mn^{II} to MnLanM was characterized using a TA Affinity ITC instrument. The ITC cell contained 60 μM MnLanM in Chelex-treated Buffer A. Titrations were carried out at 25° C., 30° C., and 37° C. For experiments at these temperatures, the titrant syringe contained 2.6 mM, 3.0 mM, or 5.0 mM MnCl₂, respectively, prepared in the same buffer. Titrations consisted of a first 0.2 L injection followed by 45×0.8 μL injections. The equilibration times were 180 s between injections, and the sample cell was stirred at 125 rpm. At each temperature, the heats of dilution were determined by titrating the same metal solutions into Buffer A without protein. The corrected heats were determined by subtracting the heats of dilution from the protein data, and the resulting data were fitted using NanoAnalyze using the “Multiple sites” model with two sets of sites, yielding for set of sites the number of binding sites (n), association constant (Kₐ), binding enthalpies (ΔH), and entropy change (ΔS). The data at 37° C. were fitted first to determine ΔH and n values and these values were used to help narrow the range of possible values for the data at 30° C. and 25° C. All parameters are shown on Table 7.

CD spectrometry. a) Preparation of low and high Mn-citrate buffers. For buffered metal titrations with MnLanM using CD spectrometry, 1 mM citrate was used as the metal-chelator buffer. The low and high Mn^{II}-citrate buffers were prepared as described. High Mn^{II}-citrate buffer was prepared by adding MnCl₂ from a 0.1 M stock solution, giving a final concentration (800 M Mn^{II}) in 50 mL. The low citrate buffer contained no Mn^{II}. b) Citrate-buffered Mn titrations with MnLanM. MnLanM was diluted to 15 μM in both high Mn^{II}-citrate and low citrate buffers. These two buffers were mixed in different ratios to provide various free Mn^{II} concentrations with a total volume of 200 μL for each sample. The blank sample was low citrate buffer without protein. Each Mn^{II}-MnLanM spectrum was measured in a 1-mm pathlength quartz CD cuvette (Jasco J/0556) at 25° C. using a Jasco J-1500 CD spectrometer. Spectra were acquired from 260 to 195 nm with the following instrument settings: 1 nm bandwidth, 0.5 nm data pitch, 50 nm/min scan rate, 4 s average time. Three scans were acquired and averaged for each condition. The blank spectrum was subtracted from each Mn^{II}-MnLanM spectrum and [Θ]_{222 nm} was plotted against free Mn^{II} concentration.

Thermal stability of MnLanM. Using a PCR machine, 30 μL of 1 mM apo-MnLanM in Buffer A was heated from 25 to 45° C. in 5° C. steps, for 10 min at each temperature, and the protein was cooled to 25° C. for 10 min. No precipitation was evident under these conditions, but in experiments including temperatures above 50° C., some precipitation was observed.

NMR spectroscopy. Samples for ¹⁷O NMR were prepared by mixing 1.22 mM apo-MnLanM with 0.248 mM MnCl₂ in Buffer A, enriched with a small amount of H₂¹⁷O. NMR spectra were acquired on a 500 MHz JEOL NMR spectrometer at temperatures ranging from 25 to 45° C. The transverse (T₂) relaxation times of ¹⁷O at 11.7 T were estimated from the full-width at half-height (Δ_{ν1/2}) of the ¹⁷O NMR linewidth [T₂˜(πΔ_{ν1/2})⁻¹], using JEOL Delta NMR Processing and Control Software v5.3.1. The ¹⁷O T₂-relaxivity

$$( {r}_{2}^{O} )$$

at each temperature was calculated by dividing the Mn^{II}-induced increase in 1/T₂ relative to that observed for the apoprotein divided by the Mn^{II} concentration in millimolar units. Although the samples were prepared using a large excess of apo-protein to minimize the presence of free Mn^{II}, estimates of unchelated Mn^{II} concentration based on thermodynamic parameters determined by ITC and FRET indicate that between 1 and 5 μM unchelated Mn^{II} is present as the temperature increases from 25 to 45° C. Because the temperature dependence on Mn^{II}

$${r}_{2}^{O}$$

is known

$$( {r}_{2 free}^{O} ) ,$$

the

$${r}_{2}^{O}$$

values of MnLanM

$$( {r}_{2 LaMP}^{O} )$$

could be isolated from the empirically observed

$${r}_{2}^{O} ( {r}_{2 emp}^{O} )$$

using Equation S1

$${r}_{2 emp}^{O} = ( x ) {r}_{2 LaMP}^{O} + ( 1 - x ) {r}_{2 free}^{O} \qquad ( Equation S1 )$$

where x corresponds to the fraction of MnLanM comprising overall Mn speciation. The values for

$${r}_{2 emp}^{O} , {r}_{2 free}^{O} , and {r}_{2 LaMP}^{O}$$

are shown in Table 8 along with the thermodynamic parameters used to estimate these values.

The

$${r}_{2 LaMP}^{O}$$

data were plotted against reciprocal temperature [1000/T (K⁻¹)] and fitted with Equations S3, S4, and S6 as described previously, using Igor 6.0.

Equations used to estimate q and water exchange parameters. The observable Mn^{II}-mediated increase in transverse relaxation rate (1/T₂ₚ) of bulk water-¹⁷O nuclei occurs predominantly through interactions between Mn^{II} and directly coordinated, rapidly exchanging water ligands as described in Equation S2:

$$\frac{1}{{T}_{p}} = \frac{1}{{T}_{2 obs}} - \frac{1}{{T}_{2 o}} = \frac{q [ Mn ]}{[ {H}_{2} O ]} \frac{1}{{T}_{2 m} + {τ}_{m}} \qquad ( Equation S2 )$$

where 1/T_{2obs} and 1/T₂ₒ are the relaxation rates of water-¹⁷O relaxation in the presence and absence of Mn^{II}, respectively, q corresponds the number of exchangeable water ligands coordinated to Mn^{II}, [Mn] and [H₂O] correspond to the concentrations of Mn^{II} and water, T₂ₘ corresponds to the time constant for transverse relaxation of water-¹⁷O directly coordinated to Mn^{II}, and τₘ corresponds to the mean residency time of the exchangeable water co-ligands.

The potency with which a given Mn^{II} complex can reduce T₂ is often described by its relaxivity

$$( {r}_{2}^{O} ) ,$$

which can be defined as the relaxation rate increase normalized to [Mn] in mM, Equation S3:

$${r}_{2}^{O} = \frac{\frac{1}{{T}_{2 obs}} - \frac{1}{{T}_{2 o}}}{[ Mn ]} = \frac{q}{[ {H}_{2} O ]} \frac{1}{{T}_{2 m} + {τ}_{m}} . \qquad ( Equation S3 )$$

The T₂ₘ term is dominated by a scalar relaxation mechanism described by Equation S4:

$$\frac{1}{{T}_{2 m}} = \frac{S ( S + 1 )}{3} {( \frac{{A}_{o}}{ℏ} )}^{2} {τ}_{sc} \qquad ( Equation S4 )$$

where S is the spin quantum number, Ao/h is the Mn-¹⁷O hyperfine coupling constant in units of rad/s, and τ_{sc} is the correlation time for scalar relaxation, Equation S5:

$$\frac{1}{{τ}_{sc}} = \frac{1}{{τ}_{m}} + \frac{1}{{T}_{1 e}} \qquad ( Equation S5 )$$

where T₁ₑ is the time constant for longitudinal electronic relaxation for Mn^{II}. For Mn^{II}, T₁ₑ increases with the square of the applied magnetic field until eventually τ_{sc}=τₘ. It has been previously demonstrated that the τ_{sc}=τₘ condition is met at the 11.7 T field strength used for this ¹⁷O NMR experiment.

The value

$${r}_{2}^{O}$$

exhibits a temperature dependence. At field strengths where τ_{sc}=τₘ, this dependence is governed by the temperature-dependent changes τₘ, described by Equation S6

$$\frac{1}{{τ}_{m}} = {k}_{ex} = \frac{{k}_{ex}^{298} T}{298} exp [ \frac{Δ {H}^{‡}}{R} ( \frac{1}{298} - \frac{1}{T} ) ] \qquad ( Equation S6 )$$

where kₑₓ²⁹⁸ refers to the water exchange rate at 298 K and ΔH^{≠} represents the enthalpy of activation for water exchange.

When T₂ₘ<τₘ,

$${r}_{2}^{O}$$

resides in the ‘slow exchange regime’ in which

$${r}_{2}^{O}$$

will increase with increasing temperature until T₂ₘ=τₘ, at which point

$${r}_{2}^{O}$$

reaches its maximum

$$( {r}_{2 m ax}^{O} ) .$$

As the temperature continues to increase, we enter the ‘fast exchange regime’ where T₂ₘ>τₘ.

When τ_{sc}=τₘ, Equations S3 and S4, can be rearranged to Equation S7:

$$q = {r}_{2 m ax}^{O} [ {H}_{2} O ] \frac{2}{\sqrt{\frac{S ( S + 1 )}{3}} ( \frac{{A}_{O}}{h} )} \qquad ( Equation S7 )$$

The value Ao/h is relatively invariant, with empirically reported values ranging between 2.6-4.2×10⁷ rad/s, and thus at sufficiently high field strength the value q can be estimated directly from

$$( {r}_{2 m ax}^{O} )$$

using Equation S8:

$$q = \frac{{r}_{2 ma x}^{O}}{510 \pm 100 {mM}^{- 1} {s}^{- 1}} \qquad ( Equation S8 )$$

where 510±100 mM⁻¹s⁻¹ corresponds to the range of

$${r}_{2 m ax}^{O}$$

per q considering the range of empirically determined Ao/h.

Construction of plasmids for constitutive sensor expression. The vector segment for the Gibson assembly reaction was PCR-amplified from pWCD0941 using the primers, pWCD Gib-1 and pWCD Gib-2. Sensor genes were PCR-amplified from pBAD-MnLaMP1 or pBAD-MnLaMP2 using the primers, pWCD Gib-3 and pWCD Gib-4. The sensor fragments were purified by gel electrophoresis (1% agarose) and ligated with the pWCD vector fragment (5:1 insert:vector) via a Gibson assembly reaction at 50° C. for 1 h following the manufacturer's protocol. For the control plasmid (pWCD-control), pWCD0941 was digested by HindIII to remove the native fluorescent protein gene and the vector fragment was purified by gel electrophoresis. The purified fragment was re-ligated using T4 ligase for 4 h at 23° C. following the manufacturer's protocol. In all cases, the ligated products were transformed into E. coli 5a cells and plated on LB agar [25 μg/mL chloramphenicol (Cm)]. Colonies were screened for the insert and the constructs were confirmed by DNA sequencing.

Inductively coupled plasma mass spectrometry (ICP-MS). a) Preparation of cell samples for quantification of total cellular Mn concentration. pWCD-control was transformed into E. coli wild type and ΔmntP and plated on LB agar (25 g/mL Cm for wild type, 25 g/mL Cm and 25 g/mL Km for ΔmntP). A single colony was inoculated in MOPS minimal medium obtaining 0.2% glucose, 0.2% casamino acids, and Cm (MOPS-Glu) for 16 h at 23° C. with 200 rpm shaking. This culture was inoculated to OD₆₀₀˜0.005 into 100 mL fresh MOPS-Glu in a 500 mL baffled flask and grown at 23° C. for 23 h with 200 rpm shaking, in order to allow sensor folding and chromophore maturation. This culture was then inoculated at OD₆₀₀=0.05 into 50 mL fresh MOPS-Glu (without casamino acids) in a 500 mL baffled flask and grown at 37° C. At OD₆₀₀˜0.2, metal stock solution (final concentration: 0 or 500 M MnCl₂) was added to the culture. At 0 (immediately before metal addition), 10, 20, 60 and 90 min, OD_{600 nm} was recorded and 10 mL of each culture was transferred to a 15-mL falcon tube on ice. At t=0, a 1 mL aliquot of the culture was also reserved on ice for cell counting. Cells were centrifuged for 1 min at 7000×g at 4° C. and the supernatant was aspirated by vacuum. The cells were gently resuspended in 1 mL ice-cold 20 mM Tris-HCl, 1 mM EDTA, pH 7.4, the cells were transferred to 1.7-mL centrifuge tubes, and they were centrifuged for 1 min at 7000×g at 4° C. again. The supernatant was aspirated and the pellet was washed twice in 1 mL of ice-cold 20 mM Tris-HCl, pH 7.4, followed by centrifugation and aspiration of the supernatant. The cell pellets were digested with 100 L Aristar Ultra HNO₃ for ˜16 h at room temperature. The digest was diluted with 7 mL 2% HNO₃ for analysis. The cellular Mn content was determined using ThermoFisher Scientific X Series II-SBM and iCAP RQ Inductively Coupled Plasma-Mass Spectrometers (ICP-MS) at the Penn State Laboratory for Isotopes and Metals in the Environment (LIME). b) Cell counting. The 1-mL aliquot of wild type and ΔmntP cells, reserved above, was diluted 10⁻⁴ to 10⁻⁷ in water and 100 μL of each dilution was plated on LB agar without antibiotics. After incubation at 37° C. for 16 h, colonies were counted and cfu/mL in the original, undiluted sample was calculated. c) Data analysis. The Mn ICP-MS value (in ppb) from the blank solution [2% (w/v) HNO₃] was subtracted from the raw ICP-MS values for each sample and, using each sample volume, converted to total moles of Mn. Total cell volume was calculated using the cell counting results, culture volume, and the estimated cellular volume (3.2 fL). Cellular Mn concentrations were calculated from the total Mn and total cell volume.

Sensor calibration in E. coli. pWCD-control, pWCD-MnLaMP1 and pWCD-MnLaMP2 were transformed into chemically competent E. coli BW25113, ΔmntP, or ΔmntR and plated on LB-agar (25 μg/mL Cm for BW25113; 25 μg/mL Cm and 25 μg/mL Km for ΔmntP and ΔmntR). A single colony was inoculated into 100 mL LB media containing 25 g/mL Cm and grown at 23° C. with 200 rpm shaking for ˜16 h. The OD_{600 nm} (2.3-2.5) was measured, and 5 mL of each culture were transferred to a 14-mL culture tube and centrifuged at 3000×g for 5 min at 25° C. The supernatant was decanted and the pellet was washed 3 times with 5 mL phosphate-free MOPS media supplemented with 0.2% glucose. After the last resuspension, the culture was diluted to OD_{600 nm}˜0.2 in 50 mL phosphate-free MOPS, 0.2% glucose, 25 μg/mL Cm, and incubated with shaking at 37° C. in a 500 mL baffle flask for 90 min. Fractions (100 μL) were removed at 0, 60, and 90 min for measurement of resting FRET ratio and OD_{600 nm}. FRET ratios were measured in half-area 96-well plates on a Biotek Synergy H1 microplate reader (433 nm excitation, 474-481 nm emission, 524-531 nm emission, 1 nm steps, 119 gain; plate reader was set at 25° C.). Once the resting FRET ratio was stabilized, 1 mM N,N,N′,N′-tetrakis(2-pyridinylmethyl)-1,2-ethanediamine (TPEN) was added into the 50 mL culture. After 10 min of further incubation, 100 μL was removed for FRET ratio measurement to establish Rₘᵢₙ. A 5-mL aliquot of the culture was transferred to a 14-mL culture tube, centrifuged, and washed 3 times with 5 mL phosphate-free MOPS media as described above, to remove TPEN. At the final resuspension, 2.5 M 4-BrA23187 and 10 mM MnCl₂ were added, the culture was returned to the shaker, and 100 μL aliquots were removed at 5, 10, 20, 30, 45, 60, and 90 min for FRET measurements with the same parameters as above to define Rₘₐₓ.

FRET ratios were calculated after subtraction of the cell background determined from the pWCD-control-expressing cells, applying the following equations at each emission wavelength:

$$Normalized {fluorescence}_{sensor} = {OD}_{600 n m , control} / {OD}_{600 n m , sensor} \cdot raw {fluorescence}_{sensor} \qquad ( Equation S9 )$$

$$Adjusted {fluorescence}_{sensor} = Normalized {fluorescence}_{sensor} - raw {fluorescence}_{control} \qquad ( Equation S10 )$$

The control and sensor-expressing cells had similar OD values but this correction improved data reproducibility. The adjusted fluorescence values for 476-480 nm emission were averaged to give the ECFP value (F_{478 nm}) and those from 526-530 nm were averaged for citrine (F_{529 nm}). The FRET ratio was calculated by F_{529 nm}/F_{478 nm}.

Metal uptake kinetics monitored by MnLaMP1 and MnLaMP2. pWCD-control, pWCD-MnLaMP1, and pWCD-MnLaMP2 were transformed into chemically competent E. coli BW25113 (WT) or variants (ΔmntP, ΔmntH, ΔmntR), which were plated on LB-agar (25 μg/mL Cm for WT; 25 μg/mL Cm and 25 μg/mL Km for variants). A single colony was inoculated into 7 mL MOPS minimal media with 0.2% glucose, 0.2% casamino acids, Cm 25 g/mL and grown at 23° C. with 200 rpm shaking for ˜16 h. The overnight culture was inoculated at OD₆₀₀˜0.005 in 100 mL MOPS/glucose/Cm/casamino acids media and grown at 23° C. for 16 h at 200 rpm. A 10-mL volume of this culture was removed and centrifuged, and the cell pellet was washed twice with MOPS/glucose/Cm media (without casamino acids). The cell suspension was used to inoculate 4×50 mL of the same media in 250 mL baffle flasks to an OD₆₀₀˜0.05. These cultures were grown with shaking at 37° C. for ˜4 h. At OD₆₀₀˜0.2, each culture was treated with 0, 100, 250, or 500 μM MnCl₂ and 100 μL was removed at 0, 5, 10, 20, 30, 45, 60 and 90 min, for plate reader measurements of OD_{600 nm} and fluorescence. FRET ratios were calculated as described above. A control experiment established that addition of 500 μM MnCl₂ did not affect the pH of the media.

For metal selectivity experiments, E. coli BW25113 cells were transformed with pWCD-MnLaMP1, pWCD-MnLaMP2 or pWCD-control and grown as described above. At OD₆₀₀˜0.2, 100 μL culture was removed as the t=0 sample, and 500 M CaCl₂), 500 M MnCl₂, 500 μM ferric ammonium citrate, 3 mM MgCl₂, 50 M ZnSO₄, or 500 M ZnSO₄ were added to the cultures. At 5, 10, 20, 30, 45, 60, and 90 min after addition, 100 μL of cell culture was removed and both OD_{600 nm} and fluorescence measurements were acquired; data analysis was performed as above.

Determination of intracellular sensor concentration. a) Cellular analysis. pWCD-MnLaMP1 was transformed into E. coli BW25113 and plated on LB-agar (25 g/mL Cm). A single colony was inoculated into 7 mL MOPS minimal media with 0.2% glucose, 0.2% casamino acids, Cm 25 μg/mL and grown at 23° C. with 200 rpm shaking for ˜16 h. The overnight culture was inoculated at OD₆₀₀˜0.005 in 100 mL MOPS/glucose/Cm/casamino acids media and grown at 23° C. for 16 h. From this culture, ˜7 mL was removed and centrifuged. The cell pellet was washed twice with MOPS/glucose/Cm media (without casamino acids). The cell resuspension was inoculated into 2×250 mL of the same media in 500-mL baffle flasks to an OD₆₀₀˜0.05. These cultures were grown at 37° C. with 200 rpm shaking for ˜4 h. The OD₆₀₀ values (˜0.2) were recorded and cells were harvested by centrifugation. The cell pellets were resuspended with 3 mL of Buffer A containing 0.4 mM PMSF and 1 protease inhibitor tablet. The cell suspension was sonicated for 10 min (3 seconds on/7 seconds off, 50% amplitude), followed by centrifugation at 40,000×g for 35 min at 4° C. The supernatant was transferred into a 5-mL Eppendorf tube and 100 μL was removed for plate reader measurement of fluorescence (433 nm excitation, 426-560 nm emission, 1 nm steps, 57 gain, 25° C.). An additional sample was prepared containing 1 mM EDTA. b) Standard curve. Purified MnLaMP1 was diluted to 4.9 μM in Buffer A. This sensor was diluted to 2.45, 0.98, and 0.49 M and fluorescence of 100 μL of each solution was measured by plate reader in parallel with the lysate samples. The fluorescence at 528 nm was plotted against sensor concentration. c) Data analysis. F528 nm of the lysate sample with 1 mM EDTA added was used to calculate the sensor concentration in the lysate using the standard curve. The intracellular sensor concentration was determined using the concentration in the lysate, the total volume of lysate, the number of cells lysed based on (6.35±1.14)×10⁸ CFU/mL/OD₆₀₀ (determined through the cell counting experiments described above), and 3.2 IL cellular volume.

Construction of sensors for mammalian cell expression. The vector fragment was amplified from pcDNA3.1 using primers, pcDNA3.1-1 and pcDNA3.1-2. The MnLaMP1 insert was amplified from pWCD-MnLaMP1 using primers pWCD Gib-3 and pWCD Gib-4. The linearized vector and the insert were combined in a Gibson assembly reaction (insert:vector ratio 5:1) at 50° C. for 1 h according to the manufacturer's protocol. The product was transformed into E. coli 5alpha cells, plated on LB agar (Amp, 100 g/mL), and grown at 37° C. overnight. The colonies were screened and the correct sequences were confirmed by DNA sequencing using CMV forward and BGH reverse primers.

For construction of the Golgi-localized sensor, the Golgi-targeting signal peptide from pcDNA-Golgi-ZapCY1 (Genbank ID JF261179.1) was ordered as a 220-bp gBlock containing 5′-NdeI and 3′-BamHI sites and digested. pBAD-MnLaMP1 was digested using NdeI and BamHI and the cut vector was purified by gel electrophoresis. The digested signal peptide was inserted into the digested vector using T4 ligase for 4 h at 23° C. following the manufacturer's protocol (5:1, insert:vector). Colonies were screened and the correct sequence was confirmed by DNA sequencing. From this plasmid, the Golgi-MnLaMP1 region was amplified using primers pcDNA3.1-4 and pcDNA3.1-5. pcDNA3.1 was amplified using primers pcDNA3.1-2 and pcDNA3.1-3. The vector and insert were combined in a Gibson assembly reaction as described above and the correct plasmid was confirmed by DNA sequencing.

FRET assay in HeLa cells. HeLa cells were grown in minimum essential medium supplemented with 10% fetal bovine serum, 100 U/mL penicillin, and 100 g/mL streptomycin. DNA transfections were performed using JetPEI reagent following the manufacturer's recommendations. One day (24 h) after transfection, cells were treated with 500 μM Mn^{II} and imaged live for 15 min before and 30 min after Mn addition. One hour after Mn addition cells were fixed and stained for GPP130 using a custom mouse monoclonal antibody. Live cell imaging was performed using a Zeiss LSM 710 confocal microscope equipped with a Plan-Apo 63×1.4 NA oil immersion objective and ZEN software. Fixed cells were imaged using a Nikon swept-field confocal microscope equipped with a 100×1.45 NA oil immersion objective and images captured using an iXon3 X3 DU897 electron-multiplying charge-coupled device camera (Andor Technology). All depicted images are maximum-intensity projections of the stacks. Image quantification was performed using NIH ImageJ. To quantify the relative intensity of a signal in one cell, average intensity projection was first generated from individual z-stacks, background was subtracted for each image by drawing a region of interest in a part of the image that did not have any cellular component, an outline of the cell was drawn, and total fluorescence per cell was measured. FRET ratio was calculated by dividing the emission of citrine by the emission of ECFP.

TABLE 3
Fluorescence response of 4P₂A-based LaMP1 variants (0.5 μM sensor)
to Mn^{II} and Ca^{II}. Each variant was titrated with solutions of MnCl₂ or
CaCl₂, or EGTA-buffered CaCl₂, at 25° C. in 30 mM MOPS, 100
mM KCl, pH 7.2. The maximum free metal concentration tested was
10 mM. Data represent mean ± SEM of 3 technical replicates.

| | Mn^{II} | | | Ca^{II} | | |
|---|---|---|---|---|---|---|
| | K_{eff} (μM) | n | F/F₀ | K_{eff} (μM) | n | F/F₀ |
| LaMP1 | 750 ± 10 | 1.9 ± 0.1 | 4.8 | 1400 ± 100 | 1.1 ± | 3.2 |
| 4P₂A | 73 ± 6 | 1.3 ± 0.2 | 4.2 | 1.5 ± 0.1, | 2.2 ± 0.1, | 3.5, 3.3 |
| | | | | 68 ± 8 | 1.2 ± 0.1 | |
| 3D₉Q/4P₂A | 260 ± 10 | 1.9 ± 0.1 | 2.8 | 36 ± 1 | 2.2 ± 0.1 | 3.2 |
| 3D₉H/4P₂A | 200 ± 10 | 1.4 ± 0.1 | 3.5 | 91 ± 2 | 1.7 ± 0.1 | 4.0 |
| 3E₁₂D/4P₂A | 79 ± 2 | 1.5 ± 0.1 | 4.3 | 150 ± 10 | 1.9 ± 0.1 | 4.3 |
| 3D₉H/3E₁₂D/4P₂A | 42 ± 7 | 1.3 ±0.2 | 2.4 | 84 ± 4 | 1.4 ± 0.1 | 2.4 |

TABLE 4
Determination of K_{eff}, Hill coefficient (n), and FRET response for each LaMP1 variant (0.5 μM sensor) to Mg^{II}, Co^{II}, and Zn^{II}.
Each variant was titrated with solutions of MgCl₂, CoCl₂, or ZnCl₂, or EGTA-buffered Zn^{II} (for 3D₉H and MnLaMP1), at 25° C. in
30 mM MOPS, 100 mM KCl, pH 7.2. The maximum free metal concentration tested was 10 mM. Data represent mean ± SEM of 3 technical replicates.

| | Mg^{II} | | | Co^{II} | | | Zn^{II} | | |
|---|---|---|---|---|---|---|---|---|---|
| | K_{eff} (μM) | n | F/F₀ | K_{eff} (μM) | n | F/F₀ | K_{eff} (μM) | n | F/F₀ |
| LaMP1 | >3000 | N.D.^{a} | N.D. | 2400 ± 300 | 2.0 ± 0.2 | 4.0 | >70 | N.D. | N.D. |
| 3D₉H | >1000 | N.D. | N.D. | 1400 ± 300 | 1.2 ± 0.2 | 1.4 | 30 ± 1 | 1.3 ± 0.4 | 1.9 |
| 3E₁₂D | >1000 | N.D. | N.D. | 1400 ± 100 | 2.5 ± 0.3 | 1.3 | >1000 | N.D. | N.D. |
| 3D₉Q/3E₁₂D | >1000 | N.D. | N.D. | 740 ± 40 | 1.4 ± 0.1 | 1.3 | >1000 | N.D. | N.D. |
| 3D₉H/3E₁₂D (MnLaMP1) | 1400 ± 200 | 1.4 ± 0.1 | 3.3 | 310 ± 10 | 1.3 ± 0.1 | 3.2 | 10 ± 1, 400 ± 10 | 2.0 ± 0.1, 1.6 ± 0.1 | 1.5, 2.0 |
| 4P₂A | >100 | N.D. | N.D. | 940 ± 40 | 1.6 ± 0.1 | 3.3 | >1000 | N.D. | N.D. |
| 3D₉H/4P₂A | >3000 | N.D. | N.D. | 1300 ± 300 | 1.2 ± 0.2 | 2.7 | 83 ± 10 | 1.3 ± 0.3 | 2.8 |
| 3E₁₂D/4P₂A | 2700 ± 200 | 2.2 ± 0.3 | 2.8 | 830 ± 30 | 2.1 ± 0.1 | 4.3 | 540 ± 90 | 0.8 ± 0.1 | 2.9 |
| 3D₉H/3E₁₂D/4P₂A | 710 ± 60 | 1.5 ± 0.2 | 1.8 | 350 ± 10 | 1.1 ± 0.1 | 2.7 | >30 | N.D. | N.D. |

^{a}N.D.: Not determined. Some response observed but it was not saturated at the highest metal concentration tested (10 mM); the K_{eff} is given as greater than the lowest concentration for which a response was observed.

TABLE 5
Determination of K_{eff}, Hill coefficient (n), and FRET response for each LaMP1 variant (0.5 μM sensor)
to Fe^{II}, Ni^{II}, and La^{III}. Each variant was titrated with unbuffered solutions of ferrous ammonium sulfate or
NiCl₂, or EDDS-buffered LaCl₃, at 25° C. in 30 mM MOPS, 100 mM KCl, pH 7.2. The titration with Fe^{II}
was performed under anaerobic conditions. Data represent mean ± SEM of 3 technical replicates.

| | Fe^{II} | | | Ni^{II} | | | La^{III} | | |
|---|---|---|---|---|---|---|---|---|---|
| | K_{eff} (μM) | n | F/F₀ | K_{eff} (μM) | n | F/F₀ | K_{eff} (nM) | n | F/F₀ |
| 3D₉H/3E₁₂D | 74 ± 6 | 1.2 ± 0.2 | 2.8 | 390 ± 20 | 1.0 ± 0.1 | 3.1 | 0.10 ± 0.01, | 1.0 ± 0.1, | 3.7, 1.2 |
| (Mn-LaMP1) | | | | | | | 6.3 ± 0.7 | 1.0 ± 0.1 | |
| 3E₁₂D/4P₂A | 640 ± 10 | 2.5 ± 0.8 | 2.9 | 1600 ± 100 | 1.6 ± 0.1 | 3.3 | 0.38 ± 0.02 | 1.0 ± 0.1 | 3.8 |

TABLE 6
Determination of K_{eff}, Hill coefficient (n), and fold change
in FRET for each D₁₂A variant of MnLaMP1 responding to Mn^{II} and
Co^{II}. FRET ratio data are shown in FIG. 13. Conditions:
0.5 μM sensor, 30 mM MOPS, 100 mM KCl, pH 7.2, 25°
C. Data represent mean ± SEM of 3 technical replicates.

| | D46A | | | D70A | | | D95A | | |
|---|---|---|---|---|---|---|---|---|---|
| | K_{eff} | | | K_{eff} | | | K_{eff} | | |
| | (μM) | n | F/F₀ | (μM) | n | F/F₀ | (μM) | n | F/F₀ |
| Mn^{II} | 36 ± | 1.1 ± | 1.6 | 29 ± | 1.6 ± | 1.9, | 40 ± | 1.0 ± | 2.7 |
| | 5 | 0.1 | | 1, | 0.1, | 1.3 | 10 | 0.2 | |
| | | | | 1400 ± | 1.7 ± | | | | |
| | | | | 100 | 0.1 | | | | |
| Co^{II} | 510 ± | 0.9 ± | 1.9 | 270 ± | 1.1 ± | 2.3 | 310 ± | 1.2 ± | 2.9 |
| | 30 | 0.1 | | 10 | 0.1 | | 10 | 0.1 | |

TABLE 7
Thermodynamic parameters (Kₐ, n, ΔH, ΔG, ΔS) for
Mn^{II} binding to 60 μM untagged MnLanM (2.6 mM at 25° C.,
3 mM at 30° C., and 5 mM Mn^{II} at 37° C., respectively)
determined by ITC. Data were fitted to a binding model with
two sets of sites. Uncertainties were determined from standard
deviations from two titrations.

| | 25° C. | | 30° C. | | 37° C. | |
|---|---|---|---|---|---|---|
| | Phase 1 | Phase 2 | Phase 1 | Phase 2 | Phase 1 | Phase 2 |
| Kₐ (mM⁻¹) | 69 ± | 65 ± | 43 ± | 36 ± | 38 ± | 25 ± |
| | 20 | 20 | 9 | 8 | 7 | 4 |
| K_{d} (μM) | 15 | 15 | 23 | 28 | 26 | 40 |
| n | 0.66 ± | 1.7 ± | 0.61 ± | 2.2 ± | 0.79 ± | 3.1 ± |
| | 0.22 | 0.3 | 0.30 | 0.4 | 0.37 | 0.4 |
| ΔH (kcal | 31 ± | −12 ± | 28 ± | −10 ± | 24 ± | −9.7 ± |
| mol⁻¹) | 8 | 4.8 | 7 | 4 | 6 | 3.5 |
| ΔG (kcal | −1.1 ± | −6.6 ± | −6.4 ± | −6.3 ± | −6.5 ± | −6.3 ± |
| mol⁻¹) | 0.3 | 3.1 | 1.7 | 2.3 | 1.7 | 2.3 |
| ΔS (cal K⁻¹ | 110 ± | −17 ± | 110 ± | −13 ± | 97 ± | −11 ± |
| mol⁻¹) | 20 | 4.7 | 10 | 1.5 | 2 | 1 |

TABLE 8
Deconvolution of contributions of MnLanM-bound and free
Mn^{II} to ¹⁷O T₂-relaxivity (r₂^{o}) in NMR studies. Samples
consisted of 1.22 mM apo-MnLanM and 0.248 mM MnCl₂ in
Buffer C, enriched with a small amount of H₂¹⁷O. The
K_{d} values were obtained from ITC experiments (Table 7),
assuming 3 independent sites with similar affinity.
with temperature dependence calculated based on van ′t
Hoff analysis (FIG. 17). The minor contribution from
unbound Mn(H₂O)₆²⁺ [free Mn] was estimated
using K_{d} = [M][L]/[ML], where M_{free} =
Mₜₒₜₐₗ − ML and L_{free} = Lₜₒₜₐₗ − ML, and
solving the resulting quadratic equation for M_{free}.
The r₂^{o} values of MnLanM (r_{2LaMP}^{o}) were isolated from
the empirically observed r₂^{o} (r₂ₑₘₚ^{o}) using Equation S1.

| | | | | | | Overall contributions | |
|---|---|---|---|---|---|---|---|
| | | | | [Mn as | | to r₂ₑₘₚ^{o} | |
| Temp | K_{d} | [binding | [free Mn] | MnLanM] | r₂ₑₘₚ^{o} | r_{2free}^{o} | r_{2LaMP}^{o} |
| (° C.) | (mM) | sites] (mM) | (mM) | (mM) | (mM⁻¹s⁻¹) | (mM⁻¹s⁻¹) | (mM⁻¹s⁻¹) |
| 10 | 0.0047 | 3.66 | 0.00034 | 0.2478 | 296 | 1 | 295 |
| 15 | 0.0073 | 3.66 | 0.00053 | 0.2477 | 454 | 3 | 451 |
| 20 | 0.011 | 3.66 | 0.00080 | 0.2475 | 588 | 5 | 583 |
| 25 | 0.017 | 3.66 | 0.0012 | 0.2472 | 810 | 10 | 800 |
| 30 | 0.025 | 3.66 | 0.0018 | 0.2468 | 878 | 17 | 861 |
| 35 | 0.036 | 3.66 | 0.0026 | 0.2462 | 1019 | 28 | 991 |
| 40 | 0.052 | 3.66 | 0.0037 | 0.2454 | 1142 | 44 | 1097 |

TABLE 9
Fluorescence response for the X₄G variants in EF hands
1-3 (0.5 μM sensor, 25° C., 100 mM KCl, 30
mM MOPS, pH 7.2). See FIG. 27. The attempt to fit the
MnLaMP1-K62G titration with two phases did not converge.
Data represent mean ± SEM of 3 technical replicates.

| Variant | K_{eff}, μM | n | F/F₀ |
|---|---|---|---|
| MnLaMP1-K38G | 12 ± 1, 1880 ± 500 | 1.5 ± 0.1, 1.3 ± 0.4 | 1.8, 1.1 |
| MnLaMP1-K62G | 30 ± 2 | 1.2 ± 0.1 | 2.4 |

TABLE 10
Primers used in this study

| Name | |
|---|---|
| Sequencing | Sequence |
| pBAD-F | 5′-ATGCCATAGCATTTTTATCC-3′ (SEQ ID NO: 12) |
| pBAD-R | 5′-GATTTAATCTGTATCAGG-3′ (SEQ ID NO: 13) |
| ECFP-mid | 5′-CAACCACTACCTGAGCAC-3′ (SEQ ID NO: 14) |
| T7P | 5′-TAATACGACTCACTATAGGG-3′ (SEQ ID NO: 15) |
| T7T | 5′-GCTAGTTATTGCTCAGCGG-3′ (SEQ ID NO: 16) |
| CMV forward | 5′-CGCAAATGGGGGGTAGGCGTG-3′ (SEQ ID NO: 17) |
| BGH reverse | 5′-TAGAAGGCACAGTCGAGG-3′ (SEQ ID NO: 18) |

Ligation/Gibson assembly

| | |
|---|---|
| Ndel-MnLanM-F | 5′-AATACATATGCCAACTACGACTACC-3′ (SEQ ID NO: 19) |
| EcoRI-MnLanM-R | 5′-AATAGAATTCTTAACGAATTAAGTTGACC-3′ (SEQ ID NO: 20) |
| pWCD Gib-1 | 5′-GCTGTCCACCAGTCATGCTAGCCATAGATCCTTTCTCCTCTTTC |
| | AGATCC-3′ (SEQ ID NO: 21) |
| pWCD Gib-2 | 5′-CGGCATGGACGAGCTATACAAGTAATAAGGATCTCCAGGCATCAAA |
| | TAAA-3′ (SEQ ID NO: 22) |
| pWCD Gib-3 | 5′-TTTATTTGATGCCTGGAGATCCTTATTACTTGTATAGCTCGTCCATG |
| | CCG-3′ (SEQ ID NO: 23) |
| pWCD Gib-4 | 5′-GGATCTGAAAGAGGAGAAAGGATCTATGGCTAGCATGACTGGTGGA |
| | CAGC-3′ (SEQ ID NO: 24) |
| pcDNA3.1-1 | 5′-GAACAGCTCCTCGCCCTTGCTCACCATGCTCGGTACCAAGCTTAAGTTT |
| [Vector, rev] | AAACGCTAG-3′ (SEQ ID NO: 25) |
| pcDNA3.1-2 | 5′-CACTCTCGGCATGGACGAGCTATACAAGTAAGAATTCTGCAGATATCCA |
| [Vector, fwd] | CACAGTGG-3′ (SEQ ID NO: 26) |
| pcDNA3.1-3 | 5′-GGAGCGGCTCCCGAAGCCTCATGCTCGGTACCAAGCTTAAGTTTAAACG |
| [Golgi-vector, rev] | CTAG-3′ (SEQ ID NO: 27) |
| pcDNA3.1-4 | 5′-GTTTAAACTTAAGCTTGGTACCGAGCATGAGGCTTCGGGAGC-3 |
| [Golgi-insert, fwd] | (SEQ ID NO: 28)′ |
| pcDNA3.1-5 | 5′-CTGTGCTGGATATCTGCAGAATTCTTACTTGTACAGCTCGTCCATGC |
| [Golgi-insert, rev] | CGAG-3′ (SEQ ID NO: 29) |

TABLE 11
Bacterial strains used in this study

| Strains | Genotype |
|---|---|
| E. coli 5α | fhuA2 Δ(argF-lacZ)U169 phoA glnV44 ϕ80 Δ(lacZ)M15 gyrA96 recA1 relA1 |
| | endA1 thi-1 hsdR17 |
| E. coli BL21(DE3) | fhuA2 [lon] ompT gal (λ DE3) [dem] ΔhsdS |
| E. coli BW25113 | λ DE3 = λ sBamHlo ΔEcoRI-B int::(lacl::PlacUV5::T7 gene1) i21 Δnin5 |
| E. coli JW5830-1 | F⁻ wild-type |
| E. coli JW2388-1 | F−, Δ(araD-araB) 567, ΔlacZ4787(::rrnB-3), λ⁻, ΔmntP745::kan, rph-1, |
| E. coli JW0801-1 | A(rhaD-rhaB)568, hsdR514 |
| | F−, Δ(araD-araB) 567, ΔlacZ4787(::rrnB-3), λ⁻, ΔmntH729::kan, rph-1, |
| | A(rhaD-rhaB)568, hsdR514 |
| | F−, Δ(araD-araB)567, ΔlacZ4787(::rrnB-3), λ⁻, ΔmntR788::kan, rph-1, |
| | Δ(rhaD-rhaB)568, hsdR514 |

TABLE 12
Plasmids used in this study

| | |
|---|---|
| Name | Notes |

Protein expression

| | |
|---|---|
| pBAD-LaMP1 | Amp^{R}; LanM inserted into Sphl/Sacl-digested |
| | pBAD-D2 |
| pBAD-D9H | Amp^{R}; LaMP1 with D43H/D67H/D92H in LanM |
| | domain |
| pBAD-D9N | Amp^{R}; LaMP1 with D43N/D67N/D92N in LanM |
| | domain |
| pBAD-D9Q | Amp^{R}; LaMP1 with D43Q/D67Q/D92Q in LanM |
| | domain |
| pBAD-D9QE12D | Amp^{R}; LaMP1 with |
| | D43Q/E46D/D67Q/E70D/D92Q/E95D in LanM |
| | domain |
| pBAD-D9HE12D | Amp^{R}; LaMP1 with |
| (MnLaMP1) | D43H/E46D/D67H/E70D/D92H/E95D in LanM |
| | domain |
| pBAD-E12D | Amp^{R}; LaMP1 with E46D/E70D/E95D in LanM |
| | domain |
| pBAD-D9HE12Q | Amp^{R}; LaMP1 with |
| | D43H/E46Q/D67H/E70Q/D92H/E95Q in LanM |
| | domain |
| pBAD-4P2A | Amp^{R}; LaMP1 with P36A/P60A/P85A/P109A in |
| | LanM domain |
| pBAD-D9H4P2A | Amp^{R}; LaMP1 with |
| | P36A/D43H/P60A/D67H/P85A/D92H/ |
| | P109A/D116H in LanM domain |
| pBAD-D9Q4P2A | Amp^{R}; LaMP1 with |
| | P36A/D43Q/P60A/D67Q/P85A/D92Q/ |
| | P109A/D116Q in LanM domain |
| pBAD-E12D4P2A | Amp^{R}; LaMP1 with |
| | P36A/E46D/P60A/E70D/P85A/E95D/ P109A in |
| | LanM domain |
| pBAD-D9HE12D4P2A | Amp^{R}; LaMP1 with |
| | P36A/D43H/E46D/P60A/D67H/E70D/ |
| | P85A/D92H/E95D/P109A in LanM domain |
| pBAD-D9HE12D- | Amp^{R}; MnLaMP1 with D46A in LanM domain |
| D46A | |
| pBAD-D9HE12D- | Amp^{R}; MnLaMP1 with D70A in LanM domain |
| D70A | |
| pBAD-D9HE12D- | Amp^{R}; MnLaMP1 with D95A in LanM domain |
| D95A | |
| pBAD-D9HE12D- | Amp^{R}; MnLaMP1 with K38G in LanM domain |
| K38G | |
| pBAD-D9HE12D- | Amp^{R}; MnLaMP1 with K62G in LanM domain |
| K62G | |
| pBAD-D9HE12D- | Amp^{R}; MnLaMP1 with N87G in LanM domain |
| N87G (MnLaMP2) | |

Metal uptake experiments in E. coli

| | |
|---|---|
| pWCD0941 | Cm^{R} |
| pWCD-control | Cm^{R}; Digested pWCD0941 with HindIII and |
| | religated |
| pWCD-MnLaMP1 | Cm^{R}; LaMP1 with |
| | D43H/E46D/D67H/E70D/D92H/E95D (MnLaMP1) |
| | inserted into pWCD0941 |
| pWCD-MnLaMP2 | Cm^{R}; MnLaMP2 inserted into pWCD |

Mammalian cell

| | |
|---|---|
| work | |
| pcDNA3.1-MnLaMP1 | Amp^{R}; MnLaMP1 inserted into pcDNA3.1 |
| pcDNA3.1-Golgi- | Amp^{R}; Golgi signal peptide-MnLaMP1 inserted into |
| MnLaMP1 | pcDNA3.1 |

TABLE 13
Calculated K_{d,M} values used for calculation of free metal ion
concentrations in buffered metal solutions in determinations of K_{eff} values of
metal-bound sensors and MnLanM.

| Chelator | Metal ion | log K | Adjusted K_{d,M} |
|---|---|---|---|
| EGTA | Ca^{II} | 10.86 | 1.94 × 10⁻⁷ |
| | Mn^{II} | 12.18 | 4.45 × 10⁻⁹ |
| | Zn^{II} | 12.6 | 2.80 × 10⁻⁹ |
| EDDS | La^{III} | 11.98 | 8.72 × 10⁻¹⁰ |
| Citric acid | Mn^{II} | 4.15 | 7.30 × 10⁻⁵ |

TABLE 14
Maximum and minimum concentrations used in metal titrations
that included both buffered and chelator-unbuffered
regimes (Mn^{II}, Ca^{II}, La^{III}, and Zn^{II}).
N.A. = not applicable.

| | | | |
|---|---|---|---|
| Mn^{II} | Ca^{II} | La^{III} | Zn^{II} |

Buffered

| | | | | |
|---|---|---|---|---|
| Min | 0.49 nM | 10 nM | 0.87 pM | 0.31 nM |
| Max | 0.44 μM | 9 μM | 0.86 μM | 0.28 μM |

Unbuffered

| | | | | |
|---|---|---|---|---|
| Min | 1.5 μM | 10 μM | N.A. | 0.5 μM |
| Max | 10 mM | 10 mM | N.A. | 10 mM |

### Example 2

This example provides a description of peptides of the present disclosure.

The following peptides were used: MnLanM3 (sequence of protein as expressed)
MnLanM3=MnLanM2 with N108D, D116H, and E119D

(SEQ ID NO: 4)
MAPTTTTKVD IAAF [symbol] ALAA GSAAFDKL [symbol]
LKGRVSEADL KKL [symbol] YLAAV EAQFKAA [symbol] L
ASPAGSALVN LIR

MnLaMP3=MnLanM3 inserted between ECFP and citrine analogously to MnLaMP1 and MnLaMP2

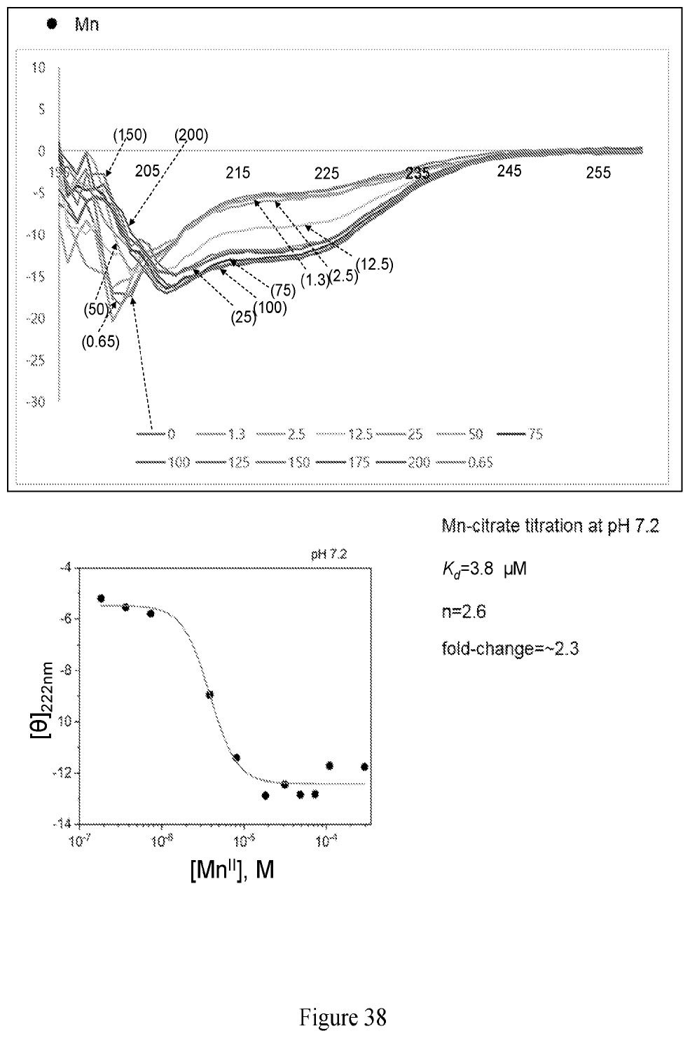

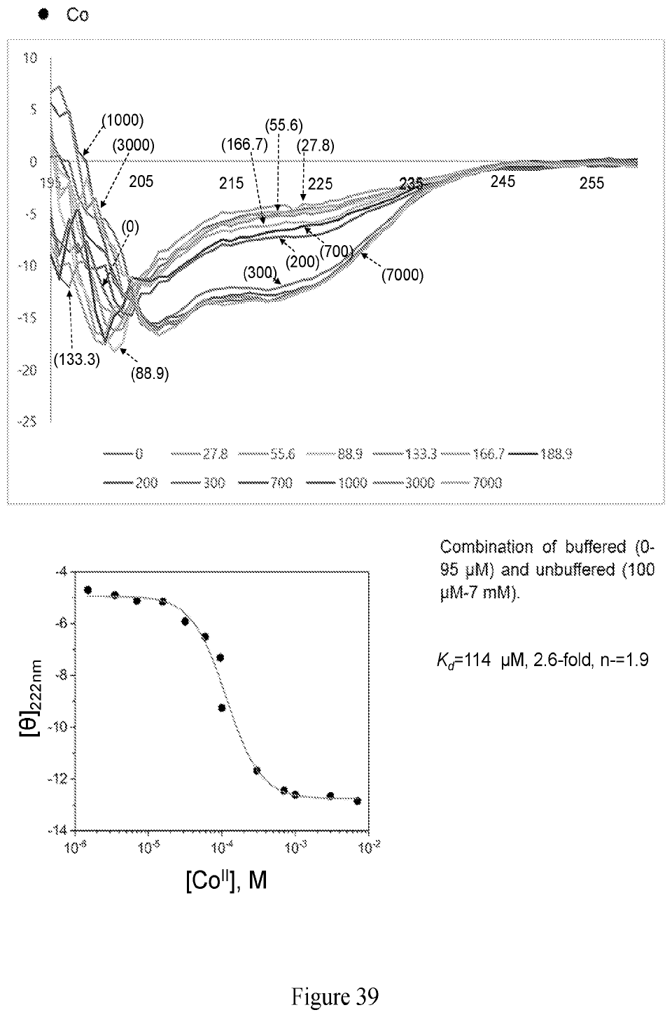

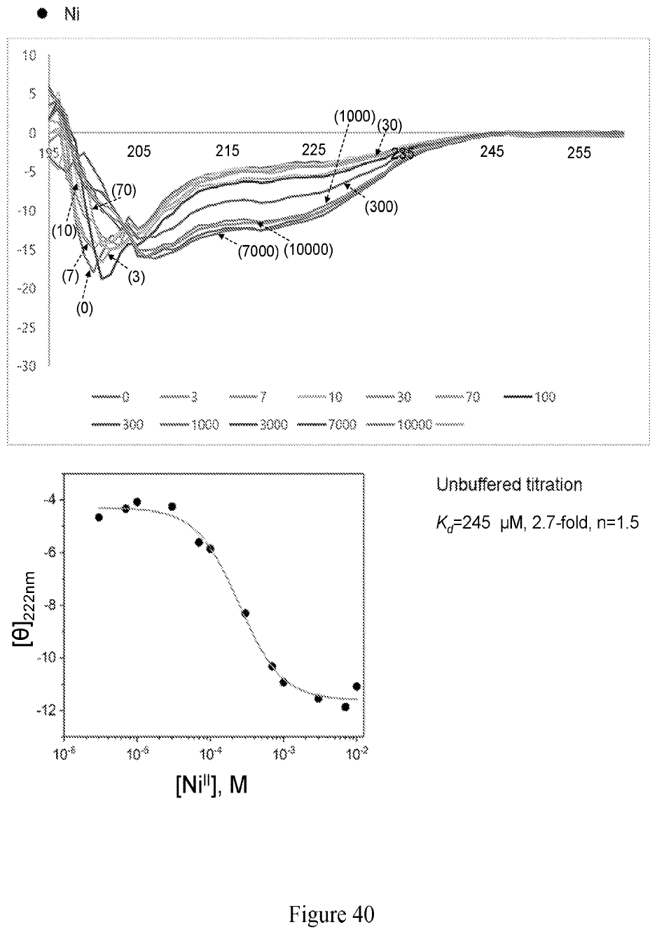

FIGS. 38 through 40 show titration data of various metals with MnLanM3. Addition of the three mutations in EF hand 4 to “activate” that EF hand for metal response leads to stronger response to Mn(II) and a 30-fold separation in K_{d,app} vs. Co(II) and 60-fold vs. Ni(II). These data suggest that the protein will likely be able to efficiently separate Mn(II) from Co(II) and Ni(II), as well as also give good separation between Co(II) and Ni(II).

MnLaMP3 shows similar K_{eff} as MnLanM3 and similar selectivity vs. Co(II), Ni(II), Mg(II), etc. as MnLaMP2.

### Example 3

This example provides a description of peptides of the present disclosure.

Construction of plasmids (Table 15), protein expression and purification, and metal titrations of MnLaMP and MnLanM variants were performed. The yields of these purified proteins are given in Table 2.

TABLE 15
Plasmids used in this Example.

| | | |
|---|---|---|
| Name | Notes | Source |

Protein expression

| | | |
|---|---|---|
| pBAD-MnLaMP3 | Amp^{R}; MnLaMP2 with N108D/ | This work |
| | D116H/E119D in MnLanM2 | |
| | domain | |
| pBAD-MnLaMP4 | Amp^{R}; MnLaMP1 with N108D/ | This work |
| | D116H/E119D in MnLanM1 | |
| | domain | |
| pET24a-MnLanM3 | Km^{R}, MnLanM2 with | This work |
| | N108D/D116H/E119D | |
| pET24a-MnLanM4 | Km^{R}, MnLanM1 with | This work |
| | N108D/D116H/E119D | |

TABLE 16

| Protein yields for each sensor and MnLanM variants | |
|---|---|
| | Protein yield |
| Name | (mg/L culture) |
| MnLaMP3 | 34 |
| MnLaMP4 | 46 |
| MnLanM3 | 34 |
| MnLanM4 | 48 |

The K_{d,M} values used for determination of free metal concentrations in each buffered metal titration sample are given in Table 17. NTA was used to buffer Mn^{II} in a range between 9.8 nM and 88 M, whereas citric acid was used for a range between 0.18 M and 292 M.

TABLE 17

| Calculated K_{d,M} values used for calculation of free Mn^{II} | | | |
|---|---|---|---|
| concentrations in buffered metal solutions, | | | |
| for determinations of K_{eff} values of metal- | | | |
| bound sensors and K_{d,app} values of MnLanM proteins. | | | |
| Chelator | pH | log K | Adjusted K_{d,M} |
| NTA | 7.2 | 5.01 | 9.81 [symbol] 10⁻⁶ |
| Citric acid | 7.2 | 4.15 | 7.30 [symbol] 10⁻⁵ |
| | 5.0 | 3.31 | 4.90 [symbol] 10⁻⁴ |

Preparation of Mn-nitrilotriacetic acid (NTA) buffered solutions. All procedures using NTA-buffered solutions should be performed in the dark because NTA is sensitive to light in solution. First, 0.0956 g of NTA was added to 40 mL of water and the pH was increased to 9-10 by addition of 6 M KOH to dissolve the solid completely. Once NTA was fully dissolved, the pH was adjusted to pH 7.2 using 6 M HCl and filled up to final volume of 50 mL with Chelex-treated water, providing a final concentration of 10 mM NTA in 50 mL water. In a 50-mL Sarstedt centrifuge tube, MOPS (0.314 g) and KCl (0.373 g) were dissolved in 35 mL water; these amounts are to provide final concentrations of 30 mM and 100 mM in 50 mL water, respectively. To this solution, 5 mL of the 10 mM NTA solution was added (1 mM final concentration). The tube was covered with aluminum foil, and 2 g Chelex-100 was added. Two separate solutions should be prepared following the protocol described above for making high Mn^{II}-NTA and low-NTA buffer. Following mixing at 4° C. for ˜12 h to prevent destabilization of NTA, the solution was adjusted to pH 7.2 using 6 M KOH. After removing the Chelex, the “high Mn^{II}-NTA” buffer (90% complexed NTA) was made by addition of a solution of 0.1 M MnCl₂ to give 900 μM Mn^{II}, followed by addition of Chelex-treated water to yield a final volume of 50 mL. The “low NTA” buffer was made by filling up to final volume of 50 mL without metal addition. These NTA buffers are stable for approximately one week.

Metal separation by ultrafiltration using MnLanM variants. All experiments were performed in 20 mM acetate, 100 mM KCl, pH 5.0 (Buffer A). Filtration used centrifugal concentrators (VivaSpin® 500) with a molecular weight cut-off of 3,000 g/mol. The filters were washed with 500 μL of Milli-Q water by centrifuging at 15,000×g for 20 min, followed by washing twice with Buffer A at 15,000×g for 20 min. Metal stock solutions (0.1 M MnCl₂, CoCl₂, and NiSO₄) were prepared in Buffer A. Protein (100 M) was prepared in 492.5 M Buffer A, and then 2.5 μL of each metal stock solution was added to yield 500 M each metal and a final volume of 500 μL. For the control sample, 2.5 μL of each metal stock solution was added to 492.5 μL Buffer A without protein. After metal addition, the samples were incubated using the nutating mixer for 10 min at 25° C., transferred to the filtration device, and centrifuged at 15,000×g for 60 min. The fraction retained above the filter (˜25 μL for the control and 50-75 μL for protein samples) was collected and the volume recorded. For ICP-MS sample preparation, the flowthrough and retentate were diluted by 10⁴-fold, and the metal stock solution was diluted by 10⁵-fold in 7 mL 2% nitric acid solution. The number of moles of metal in each sample was calculated using each sample volume. The M/M₀ ratio, which represents the ratio of the metal in the retentate to the total metal added, was calculated by dividing the number of moles of metal in the retentate by the sum of that in both the retentate and flowthrough, as described below an equation:

$$M / {M}_{0} = \frac{moles of {M}_{retentate}^{II}}{moles of {M}_{retentate}^{II} + moles of {M}_{flowthrough}^{II}}$$

Immobilization of MnLanM4 to the agarose beads. A Cys-containing version of MnLanM4 (MnLanM4-Cys) with the sequence given below was constructed, purified, and immobilized analogously to prior work with other LanMs. The Cys-containing ortholog of MnLanM4 has the following sequence:

(SEQ ID NO: 30)
MPTTTTKVDIAAFDPDKDGTIHLKDALAAGSAAFDKLDPDKDGTLHAKD
LKGRVSEADLKKLDPDNDGTLHKKDYLAAVEAQFKAADPDNDGTIHARD
LASPAGSALVNLIRGSGC,
which has the corresponding DNA sequence:
(SEQ ID NO: 31)
ATGCCAACTACGACTACCAAAGTTGATATCGCGGCGTTTGACCCGGACA
AAGATGGGACCATCCACCTGAAAGACGCTTTGGCGGCAGGTTCCGCGGC
CTTCGACAAGTTGGACCCGGATAAAGATGGTACTCTGCACGCCAAAGAC
CTGAAGGGCCGCGTGTCTGAGGCAGACCTTAAGAAGCTGGACCCGGACA
ATGACGGAACCCTGCACAAGAAAGACTACTTAGCAGCGGTAGAGGCACA
GTTTAAGGCCGCTGACCCTGACAACGATGGCACTATTCACGCCCGTGAC
CTTGCAAGCCCAGCGGGGTCGGCCCTGGTCAACTTAATTCGTGGCAGCG
GCTGCTAA.

The immobilization efficiency of MnLanM4 was calculated to be 99.7%.

Breakthrough experiment of MnLanM4 with Mn^{II}. The immobilized MnLanM4 was washed with 5 CV (column volume) HCl, 5 CV H₂O, and then 5 CV 10 mM PIPES, pH 5.0 buffer before breakthrough experiment with Mn^{II}. ˜0.1 M MnCl₂ was diluted to 200 M MnCl₂ in 10 mM PIPES, pH 5.0 buffer. The MnCl₂ solution was pumped at 1.5 mL/min rate and 45 CV was collected in 1.0 mL aliquots. After adsorption, the column was washed with 5 CV of H₂O before desorption. For desorption, 25 mM HCl, pH 1.8 was used by collecting 30 CV in 1.0 mL aliquots. The Mn concentration in each eluent was determined by ICP-MS.

### Example 4

This example provides a DNA sequences for preparing peptides of the present disclosure.

DNA sequences used were:

>MnLaMP1:
(SEQ ID NO: 32)
ATGGTGAGCAAGGGCGAGGAGCTGTTCACCGGGGTGGTGCCCATCCTGGTCGAG
CTGGACGGCGACGTAAACGGCCACAGGTTCAGCGTGTCCGGCGAGGGCGAGGGC
GATGCCACCTACGGCAAGCTGACCCTGAAGTTCATCTGCACCACCGGCAAGCTG
CCCGTGCCCTGGCCCACCCTCGTGACCACCCTGACCTGGGGCGTGCAGTGCTTCA
GCCGCTACCCCGACCACATGAAGCAGCACGACTTCTTCAAGTCCGCCATGCCCGA
AGGCTACGTCCAGGAGCGTACCATCTTCTTCAAGGACGACGGCAACTACAAGAC
CCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACAA
CTACATCAGCCACAACGTCTATATCACCGCCGACAAGCAGAAGAACGGCATCAA
GGCCCACTTCAAGATCCGCCACAACATCGAGGACGGCAGCGTGCAGCTCGCCGA
CCACTACCAGCAGAACACCCCCATCGGCGACGGCCCCGTGCTGCTGCCCGACAA
CCACTACCTGAGCACCCAGTCCGCCCTGAGCAAAGACCCCAACGAGAAGCGCGA
TCACATGGTCCTGCTGGAGTTCGTGACCGCCGCCCGCATGCCAACTACGACTACC
AAAGTTGATATCGCGGCGTTTGACCCGGACAAAGATGGGACCATCCACCTGAAA
GACGCTTTGGCGGCAGGTTCCGCGGCCTTCGACAAGTTGGACCCGGATAAAGAT
GGTACTCTGCACGCCAAAGACCTGAAGGGCCGCGTGTCTGAGGCAGACCTTAAG
AAGCTGGACCCGGACAATGACGGAACCCTGCACAAGAAAGACTACTTAGCAGCG
GTAGAGGCACAGTTTAAGGCCGCTAACCCTGACAACGATGGCACTATTGACGCC
CGTGAACTTGCAAGCCCAGCGGGGTCGGCCCTGGTCAACTTAATTCGTGAGCTCA
TGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGCC
CGTGCCCTGGCCCACCCTCGTGACCACCTTCGGCTACGGCCTGATGTGCTTCGCC
CGCTACCCCGACCACATGAAGCAGCACGACTTCTTCAAGTCCGCCATGCCCGAA
GGCTACGTCCAGGAGCGCACCATCTTCTTCAAGGACGACGGCAACTACAAGACC
CGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAAC
TACAACAGCCACAACGTCTATATCATGGCCGACAAGCAGAAGAACGGCATCAAG
GTGAACTTCAAGATCCGCCACAACATCGAGGACGGCAGCGTGCAGCTCGCCGAC
CACTACCAGCAGAACACCCCCATCGGCGACGGCCCCGTGCTGCTGCCCGACAAC
CACTACCTGAGCTACCAGTCCGCCCTGAGCAAAGACCCCAACGAGAAGCGCGAT
CACATGGTCCTGCTGGAGTTCGTGACCGCCGCCGGGATCACTCTCGGCATGGACG
AGCTATACAAGTAA;
>MnLaMP2:
(SEQ ID NO: 33)
ATGGTGAGCAAGGGCGAGGAGCTGTTCACCGGGGTGGTGCCCATCCTGGTCGAG
CTGGACGGCGACGTAAACGGCCACAGGTTCAGCGTGTCCGGCGAGGGCGAGGGC
GATGCCACCTACGGCAAGCTGACCCTGAAGTTCATCTGCACCACCGGCAAGCTG
CCCGTGCCCTGGCCCACCCTCGTGACCACCCTGACCTGGGGCGTGCAGTGCTTCA
GCCGCTACCCCGACCACATGAAGCAGCACGACTTCTTCAAGTCCGCCATGCCCGA
AGGCTACGTCCAGGAGCGTACCATCTTCTTCAAGGACGACGGCAACTACAAGAC
CCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACAA
CTACATCAGCCACAACGTCTATATCACCGCCGACAAGCAGAAGAACGGCATCAA
GGCCCACTTCAAGATCCGCCACAACATCGAGGACGGCAGCGTGCAGCTCGCCGA
CCACTACCAGCAGAACACCCCCATCGGCGACGGCCCCGTGCTGCTGCCCGACAA
CCACTACCTGAGCACCCAGTCCGCCCTGAGCAAAGACCCCAACGAGAAGCGCGA
TCACATGGTCCTGCTGGAGTTCGTGACCGCCGCCCGCATGCCAACTACGACTACC
AAAGTTGATATCGCGGCGTTTGACCCGGACAAAGATGGGACCATCCACCTGAAA
GACGCTTTGGCGGCAGGTTCCGCGGCCTTCGACAAGTTGGACCCGGATAAAGAT
GGTACTCTGCACGCCAAAGACCTGAAGGGCCGCGTGTCTGAGGCAGACCTTAAG
AAGCTGGACCCGGACGGTGACGGAACCCTGCACAAGAAAGACTACTTAGCAGCG
GTAGAGGCACAGTTTAAGGCCGCTAACCCTGACAACGATGGCACTATTGACGCC
CGTGAACTTGCAAGCCCAGCGGGGTCGGCCCTGGTCAACTTAATTCGTGAGCTCA
TGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGCC
CGTGCCCTGGCCCACCCTCGTGACCACCTTCGGCTACGGCCTGATGTGCTTCGCC
CGCTACCCCGACCACATGAAGCAGCACGACTTCTTCAAGTCCGCCATGCCCGAA
GGCTACGTCCAGGAGCGCACCATCTTCTTCAAGGACGACGGCAACTACAAGACC
CGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAAC
TACAACAGCCACAACGTCTATATCATGGCCGACAAGCAGAAGAACGGCATCAAG
GTGAACTTCAAGATCCGCCACAACATCGAGGACGGCAGCGTGCAGCTCGCCGAC
CACTACCAGCAGAACACCCCCATCGGCGACGGCCCCGTGCTGCTGCCCGACAAC
CACTACCTGAGCTACCAGTCCGCCCTGAGCAAAGACCCCAACGAGAAGCGCGAT
CACATGGTCCTGCTGGAGTTCGTGACCGCCGCCGGGATCACTCTCGGCATGGACG
AGCTATACAAGTAA;
>MnLaMP3:
(SEQ ID NO: 34)
ATGGTGAGCAAGGGCGAGGAGCTGTTCACCGGGGTGGTGCCCATCCTGGTCGAG
CTGGACGGCGACGTAAACGGCCACAGGTTCAGCGTGTCCGGCGAGGGCGAGGGC
GATGCCACCTACGGCAAGCTGACCCTGAAGTTCATCTGCACCACCGGCAAGCTG
CCCGTGCCCTGGCCCACCCTCGTGACCACCCTGACCTGGGGCGTGCAGTGCTTCA
GCCGCTACCCCGACCACATGAAGCAGCACGACTTCTTCAAGTCCGCCATGCCCGA
AGGCTACGTCCAGGAGCGTACCATCTTCTTCAAGGACGACGGCAACTACAAGAC
CCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACAA
CTACATCAGCCACAACGTCTATATCACCGCCGACAAGCAGAAGAACGGCATCAA
GGCCCACTTCAAGATCCGCCACAACATCGAGGACGGCAGCGTGCAGCTCGCCGA
CCACTACCAGCAGAACACCCCCATCGGCGACGGCCCCGTGCTGCTGCCCGACAA
CCACTACCTGAGCACCCAGTCCGCCCTGAGCAAAGACCCCAACGAGAAGCGCGA
TCACATGGTCCTGCTGGAGTTCGTGACCGCCGCCCGCATGCCAACTACGACTACC
AAAGTTGATATCGCGGCGTTTGACCCGGACAAAGATGGGACCATCCACCTGAAA
GACGCTTTGGCGGCAGGTTCCGCGGCCTTCGACAAGTTGGACCCGGATAAAGAT
GGTACTCTGCACGCCAAAGACCTGAAGGGCCGCGTGTCTGAGGCAGACCTTAAG
AAGCTGGACCCGGACGGTGACGGAACCCTGCACAAGAAAGACTACTTAGCAGCG
GTAGAGGCACAGTTTAAGGCCGCTGACCCTGACAACGATGGCACTATTCACGCC
CGTGACCTTGCAAGCCCAGCGGGGTCGGCCCTGGTCAACTTAATTCGTGAGCTCA
TGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGCC
CGTGCCCTGGCCCACCCTCGTGACCACCTTCGGCTACGGCCTGATGTGCTTCGCC
CGCTACCCCGACCACATGAAGCAGCACGACTTCTTCAAGTCCGCCATGCCCGAA
GGCTACGTCCAGGAGCGCACCATCTTCTTCAAGGACGACGGCAACTACAAGACC
CGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAAC
TACAACAGCCACAACGTCTATATCATGGCCGACAAGCAGAAGAACGGCATCAAG
GTGAACTTCAAGATCCGCCACAACATCGAGGACGGCAGCGTGCAGCTCGCCGAC
CACTACCAGCAGAACACCCCCATCGGCGACGGCCCCGTGCTGCTGCCCGACAAC
CACTACCTGAGCTACCAGTCCGCCCTGAGCAAAGACCCCAACGAGAAGCGCGAT
CACATGGTCCTGCTGGAGTTCGTGACCGCCGCCGGGATCACTCTCGGCATGGACG
AGCTATACAAGTAA;
>MnLaMP4:
(SEQ ID NO: 35)
ATGGTGAGCAAGGGCGAGGAGCTGTTCACCGGGGTGGTGCCCATCCTGGTCGAG
CTGGACGGCGACGTAAACGGCCACAGGTTCAGCGTGTCCGGCGAGGGCGAGGGC
GATGCCACCTACGGCAAGCTGACCCTGAAGTTCATCTGCACCACCGGCAAGCTG
CCCGTGCCCTGGCCCACCCTCGTGACCACCCTGACCTGGGGCGTGCAGTGCTTCA
GCCGCTACCCCGACCACATGAAGCAGCACGACTTCTTCAAGTCCGCCATGCCCGA
AGGCTACGTCCAGGAGCGTACCATCTTCTTCAAGGACGACGGCAACTACAAGAC
CCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACAA
CTACATCAGCCACAACGTCTATATCACCGCCGACAAGCAGAAGAACGGCATCAA
GGCCCACTTCAAGATCCGCCACAACATCGAGGACGGCAGCGTGCAGCTCGCCGA
CCACTACCAGCAGAACACCCCCATCGGCGACGGCCCCGTGCTGCTGCCCGACAA
CCACTACCTGAGCACCCAGTCCGCCCTGAGCAAAGACCCCAACGAGAAGCGCGA
TCACATGGTCCTGCTGGAGTTCGTGACCGCCGCCCGCATGCCAACTACGACTACC
AAAGTTGATATCGCGGCGTTTGACCCGGACAAAGATGGGACCATCCACCTGAAA
GACGCTTTGGCGGCAGGTTCCGCGGCCTTCGACAAGTTGGACCCGGATAAAGAT
GGTACTCTGCACGCCAAAGACCTGAAGGGCCGCGTGTCTGAGGCAGACCTTAAG
AAGCTGGACCCGGACAATGACGGAACCCTGCACAAGAAAGACTACTTAGCAGCG
GTAGAGGCACAGTTTAAGGCCGCTGACCCTGACAACGATGGCACTATTCACGCC
CGTGACCTTGCAAGCCCAGCGGGGTCGGCCCTGGTCAACTTAATTCGTGAGCTCA
TGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGCC
CGTGCCCTGGCCCACCCTCGTGACCACCTTCGGCTACGGCCTGATGTGCTTCGCC
CGCTACCCCGACCACATGAAGCAGCACGACTTCTTCAAGTCCGCCATGCCCGAA
GGCTACGTCCAGGAGCGCACCATCTTCTTCAAGGACGACGGCAACTACAAGACC
CGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAAC
TACAACAGCCACAACGTCTATATCATGGCCGACAAGCAGAAGAACGGCATCAAG
GTGAACTTCAAGATCCGCCACAACATCGAGGACGGCAGCGTGCAGCTCGCCGAC
CACTACCAGCAGAACACCCCCATCGGCGACGGCCCCGTGCTGCTGCCCGACAAC
CACTACCTGAGCTACCAGTCCGCCCTGAGCAAAGACCCCAACGAGAAGCGCGAT
CACATGGTCCTGCTGGAGTTCGTGACCGCCGCCGGGATCACTCTCGGCATGGACG
AGCTATACAAGTAA;
>MnLanM1:
(SEQ ID NO: 36)
ATGCCAACTACGACTACCAAAGTTGATATCGCGGCGTTTGACCCGGACAAAGAT
GGGACCATCCACCTGAAAGACGCTTTGGCGGCAGGTTCCGCGGCCTTCGACAAG
TTGGACCCGGATAAAGATGGTACTCTGCACGCCAAAGACCTGAAGGGCCGCGTG
TCTGAGGCAGACCTTAAGAAGCTGGACCCGGACAATGACGGAACCCTGCACAAG
AAAGACTACTTAGCAGCGGTAGAGGCACAGTTTAAGGCCGCTAACCCTGACAAC
GATGGCACTATTGACGCCCGTGAACTTGCAAGCCCAGCGGGGTCGGCCCTGGTC
AACTTAATTCGTTAA;
>MnLanM2:
(SEQ ID NO: 37)
ATGCCAACTACGACTACCAAAGTTGATATCGCGGCGTTTGACCCGGACAAAGAT
GGGACCATCCACCTGAAAGACGCTTTGGCGGCAGGTTCCGCGGCCTTCGACAAG
TTGGACCCGGATAAAGATGGTACTCTGCACGCCAAAGACCTGAAGGGCCGCGTG
TCTGAGGCAGACCTTAAGAAGCTGGACCCGGACGGTGACGGAACCCTGCACAAG
AAAGACTACTTAGCAGCGGTAGAGGCACAGTTTAAGGCCGCTAACCCTGACAAC
GATGGCACTATTGACGCCCGTGAACTTGCAAGCCCAGCGGGGTCGGCCCTGGTC
AACTTAATTCGTTAA;
>MnLanM3:
(SEQ ID NO: 38)
ATGCCAACTACGACTACCAAAGTTGATATCGCGGCGTTTGACCCGGACAAAGAT
GGGACCATCCACCTGAAAGACGCTTTGGCGGCAGGTTCCGCGGCCTTCGACAAG
TTGGACCCGGATAAAGATGGTACTCTGCACGCCAAAGACCTGAAGGGCCGCGTG
TCTGAGGCAGACCTTAAGAAGCTGGACCCGGACGGTGACGGAACCCTGCACAAG
AAAGACTACTTAGCAGCGGTAGAGGCACAGTTTAAGGCCGCTGACCCTGACAAC
GATGGCACTATTCACGCCCGTGACCTTGCAAGCCCAGCGGGGTCGGCCCTGGTCA
ACTTAATTCGTTAA;
and
>MnLanM4:
(SEQ ID NO:39)
ATGCCAACTACGACTACCAAAGTTGATATCGCGGCGTTTGACCCGGACAAAGAT
GGGACCATCCACCTGAAAGACGCTTTGGCGGCAGGTTCCGCGGCCTTCGACAAG
TTGGACCCGGATAAAGATGGTACTCTGCACGCCAAAGACCTGAAGGGCCGCGTG
TCTGAGGCAGACCTTAAGAAGCTGGACCCGGACAATGACGGAACCCTGCACAAG
AAAGACTACTTAGCAGCGGTAGAGGCACAGTTTAAGGCCGCTGACCCTGACAAC
GATGGCACTATTCACGCCCGTGACCTTGCAAGCCCAGCGGGGTCGGCCCTGGTCA
ACTTAATTCGTTAA.

Although the present disclosure has been described with respect to one or more particular embodiments and/or examples, it will be understood that other embodiments and/or examples of the present disclosure may be made without departing from the scope of the present disclosure.

1. A protein capable of binding Mn^{II}, comprising the following sequence:
(SEQ ID NO: 1)
Z¹-MPTTTTKVDIAAFDPDKDGTIHLKDALAAGSAAFDKLDPDKDGTLH
AKDLKGRVSEADLKKLDPDX¹DGTLHKKDYLAAVEAQFKAAX²PDNDGTI
X³ARX⁴LASPAGSALVNLIR-X⁵-Z²,
wherein
Z¹ and Z² are optional and are a Forster resonance energy transfer (FRET) pair;
X¹ is N or G;
X² is N or D;
X³ is D or H,
X⁴ is E or D, and
X⁵ optional and is the peptide sequence GSGC (SEQ ID NO:40)
wherein when Z¹ is a FRET donor, Z² is a FRET acceptor and when Z¹ is a FRET acceptor, Z² is a FRET donor, and when X⁵ is present, then Z¹ and Z² are absent; or a protein having at least 75% identity to the residues other than X¹, X², X³, X⁴, or X⁵.

2. The protein according to claim 1, wherein the protein comprises the following sequence:
>MnLanM1:
(SEQ ID NO: 2)
MPTTTTKVDIAAFDPDKDGTIHLKDALAAGSAAFDKLDPDKDGTLHAKD
LKGRVSEADLKKLDPDNDGTLHKKDYLAAVEAQFKAANPDNDGTIDARE
LASPAGSALVNLIR;
>MnLanM2:
(SEQ ID NO: 3)
MPTTTTKVDIAAFDPDKDGTIHLKDALAAGSAAFDKLDPDKDGTLHAKD
LKGRVSEADLKKLDPDGDGTLHKKDYLAAVEAQFKAANPDNDGTIDARE
LASPAGSALVNLIR;
>MnLanM3:
(SEQ ID NO: 4)
MPTTTTKVDIAAFDPDKDGTIHLKDALAAGSAAFDKLDPDKDGTLHAKD
LKGRVSEADLKKLDPDGDGTLHKKDYLAAVEAQFKAADPDNDGTIHARD
LASPAGSALVNLIR;
>MnLanM4:
(SEQ ID NO: 5)
MPTTTTKVDIAAFDPDKDGTIHLKDALAAGSAAFDKLDPDKDGTLHAKD
LKGRVSEADLKKLDPDNDGTLHKKDYLAAVEAQFKAADPDNDGTIHARD
LASPAGSALVNLIR;
or
MnLanM4-Cys:
(SEQ ID NO: 30)
MPTTTTKVDIAAFDPDKDGTIHLKDALAAGSAAFDKLDPDKDGTLHAKD
LKGRVSEADLKKLDPDNDGTLHKKDYLAAVEAQFKAADPDNDGTIHARD
LASPAGSALVNLIRGSGC.

3. The protein according to claim 1, wherein the FRET pair is protein/peptide-based.

4. The protein according to claim 3, wherein the FRET pair is a yellow fluorescent protein-based and cyan fluorescent protein-based FRET pair.

5. The protein according to claim 4, wherein Z¹ is a cyan fluorescent protein-based group and Z² is a yellow fluorescent protein-based FRET group.

6. The protein according to claim 5, wherein Z¹ has the following sequence:
(SEQ ID NO: 6)
MVSKGEELFTGVVPILVELDGDVNGHRFSVSGEGEGDATYGKLTLKFIC
TTGKLPVPWPTLVTTLTWGVQCFSRYPDHMKQHDFFKSAMPEGYVQERT
IFFKDDGNYKTRAEVKFEGDTLVNRIELKGIDFKEDGNILGHKLEYNYI
SHNVYITADKQKNGIKAHFKIRHNIEDGSVQLADHYQQNTPIGDGPVLL
PDNHYLSTQSALSKDPNEKRDHMVLLEFVTAAR.

7. The protein according to claim 5, wherein Z² has the following sequence:
(SEQ ID NO: 7)
ELMVSKGEELFTGVVPILVELDGDVNGHKFSVSGEGEGDATYGKLTLKF
ICTTGKLPVPWPTLVTTFGYGLMCFARYPDHMKQHDFFKSAMPEGYVQE
RTIFFKDDGNYKTRAEVKFEGDTLVNRIELKGIDFKEDGNILGHKLEYN
YNSHNVYIMADKQKNGIKVNFKIRHNIEDGSVQLADHYQQNTPIGDGPV
LLPDNHYLSYQSALSKDPNEKRDHMVLLEFVTAAGITLGMDELYK.

8. The protein according to claim 1, wherein the protein has the following sequence:
>MnLaMP1:
(SEQ ID NO: 8)
MVSKGEELFTGVVPILVELDGDVNGHRFSVSGEGEGDATYGKLTLKFICTTGKLPVP
WPTLVTTLTWGVQCFSRYPDHMKQHDFFKSAMPEGYVQERTIFFKDDGNYKTRAE
VKFEGDTLVNRIELKGIDFKEDGNILGHKLEYNYISHNVYITADKQKNGIKAHFKIRH
NIEDGSVQLADHYQQNTPIGDGPVLLPDNHYLSTQSALSKDPNEKRDHMVLLEFVTA
ARMPTTTTKVDIAAFDPDKDGTIHLKDALAAGSAAFDKLDPDKDGTLHAKDLKGRV
SEADLKKLDPDNDGTLHKKDYLAAVEAQFKAANPDNDGTIDARELASPAGSALVNL
IRELMVSKGEELFTGVVPILVELDGDVNGHKFSVSGEGEGDATYGKLTLKFICTTGK
LPVPWPTLVTTFGYGLMCFARYPDHMKQHDFFKSAMPEGYVQERTIFFKDDGNYKT
RAEVKFEGDTLVNRIELKGIDFKEDGNILGHKLEYNYNSHNVYIMADKQKNGIKVNF
KIRHNIEDGSVQLADHYQQNTPIGDGPVLLPDNHYLSYQSALSKDPNEKRDHMVLLE
FVTAAGITLGMDELYK;
>MnLaMP2:
(SEQ ID NO: 9)
MVSKGEELFTGVVPILVELDGDVNGHRFSVSGEGEGDATYGKLTLKFICTTGKLPVP
WPTLVTTLTWGVQCFSRYPDHMKQHDFFKSAMPEGYVQERTIFFKDDGNYKTRAE
VKFEGDTLVNRIELKGIDFKEDGNILGHKLEYNYISHNVYITADKQKNGIKAHFKIRH
NIEDGSVQLADHYQQNTPIGDGPVLLPDNHYLSTQSALSKDPNEKRDHMVLLEFVTA
ARMPTTTTKVDIAAFDPDKDGTIHLKDALAAGSAAFDKLDPDKDGTLHAKDLKGRV
SEADLKKLDPDGDGTLHKKDYLAAVEAQFKAANPDNDGTIDARELASPAGSALVNL
IRELMVSKGEELFTGVVPILVELDGDVNGHKFSVSGEGEGDATYGKLTLKFICTTGK
LPVPWPTLVTTFGYGLMCFARYPDHMKQHDFFKSAMPEGYVQERTIFFKDDGNYKT
RAEVKFEGDTLVNRIELKGIDFKEDGNILGHKLEYNYNSHNVYIMADKQKNGIKVNF
KIRHNIEDGSVQLADHYQQNTPIGDGPVLLPDNHYLSYQSALSKDPNEKRDHMVLLE
FVTAAGITLGMDELYK;
>MnLaMP3:
(SEQ ID NO: 10)
MVSKGEELFTGVVPILVELDGDVNGHRFSVSGEGEGDATYGKLTLKFICTTGKLPVP
WPTLVTTLTWGVQCFSRYPDHMKQHDFFKSAMPEGYVQERTIFFKDDGNYKTRAE
VKFEGDTLVNRIELKGIDFKEDGNILGHKLEYNYISHNVYITADKQKNGIKAHFKIRH
NIEDGSVQLADHYQQNTPIGDGPVLLPDNHYLSTQSALSKDPNEKRDHMVLLEFVTA
ARMPTTTTKVDIAAFDPDKDGTIHLKDALAAGSAAFDKLDPDKDGTLHAKDLKGRV
SEADLKKLDPDGDGTLHKKDYLAAVEAQFKAADPDNDGTIHARDLASPAGSALVN
LIRELMVSKGEELFTGVVPILVELDGDVNGHKFSVSGEGEGDATYGKLTLKFICTTG
KLPVPWPTLVTTFGYGLMCFARYPDHMKQHDFFKSAMPEGYVQERTIFFKDDGNY
KTRAEVKFEGDTLVNRIELKGIDFKEDGNILGHKLEYNYNSHNVYIMADKQKNGIKV
NFKIRHNIEDGSVQLADHYQQNTPIGDGPVLLPDNHYLSYQSALSKDPNEKRDHMVL
LEFVTAAGITLGMDELYK;
or
>MnLaMP4:
(SEQ ID NO: 11)
MVSKGEELFTGVVPILVELDGDVNGHRFSVSGEGEGDATYGKLTLKFICTTGKLPVP
WPTLVTTLTWGVQCFSRYPDHMKQHDFFKSAMPEGYVQERTIFFKDDGNYKTRAE
VKFEGDTLVNRIELKGIDFKEDGNILGHKLEYNYISHNVYITADKQKNGIKAHFKIRH
NIEDGSVQLADHYQQNTPIGDGPVLLPDNHYLSTQSALSKDPNEKRDHMVLLEFVTA
ARMPTTTTKVDIAAFDPDKDGTIHLKDALAAGSAAFDKLDPDKDGTLHAKDLKGRV
SEADLKKLDPDNDGTLHKKDYLAAVEAQFKAADPDNDGTIHARDLASPAGSALVN
LIRELMVSKGEELFTGVVPILVELDGDVNGHKFSVSGEGEGDATYGKLTLKFICTTG
KLPVPWPTLVTTFGYGLMCFARYPDHMKQHDFFKSAMPEGYVQERTIFFKDDGNY
KTRAEVKFEGDTLVNRIELKGIDFKEDGNILGHKLEYNYNSHNVYIMADKQKNGIKV
NFKIRHNIEDGSVQLADHYQQNTPIGDGPVLLPDNHYLSYQSALSKDPNEKRDHMVL
LEFVTAAGITLGMDELYK.

9. A composition comprising a protein according to claim 1 and a carrier.

10. A composition according to claim 9, wherein the carrier is a pharmaceutically acceptable carrier.

11. A method for binding and/or detecting Mn^{II} in a sample, comprising:
contacting the sample with a protein according to claim 1, and
measuring fluorescence activity; and
wherein a change in fluorescence is used to determine whether Mn^{II} is bound to the protein.

12. The method of claim 11, wherein the protein has Z¹ and Z² groups.

13. The method of claim 11, wherein the protein is immobilized on a substrate.

14. The method according to claim 11, wherein the method further comprises separating and isolating the Mn^{II}-bound protein from the sample.

15. The method according to claim 11, wherein the method further comprises imaging.

16. A method for determining the presence or absence of Mn^{II} in a subject, comprising:
administering a protein according to claim 1 to the subject; and
measuring fluorescence activity,
wherein a change in fluorescence is used to determine whether Mn^{II} is bound to the protein.

17. The method according to claim 16, wherein the method further comprises imaging.

18. The method according to claim 16, wherein the subject is a human or non-human.

19. A device comprising a protein according to claim 1.

20. A kit comprising a protein according to claim 1.
